SEQUENCE LISTING (63 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 3 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 4 - protein, 54 aa, Homo sapiens
PDASVNFSEFSKKCSERWKTMSAKEKGKFEDMAKADKARYEREMKTYIPPKGET

SEQ ID NO: 5 - protein, 69 aa, Homo sapiens
NAPKRPPSAFFLFCSEYRPKIKGEHPGLSIGDVAKKLGEMWNNTAADDKQPYEKKAAKLKEKYEKDIAA

SEQ ID NO: 6 - protein, 216 aa, Homo sapiens [sequence omitted]

SEQ ID NO: 7 - protein, 77 aa, Homo sapiens
PTGKMSSYAFFVQTCREEHKKKHPDASVNFSEFSKKCSERWKTMSAKEKGKFEDMAKADKARYEREMKTYIPPKGET

SEQ ID NO: 8 - protein, 74 aa, Homo sapiens
FKDPNAPKRLPSAFFLFCSEYRPKIKGEHPGLSIGDVAKKLGEMWNNTAADDKQPYEKKAAKLKEKYEKDIAAY

SEQ ID NO: 9 - DNA, 648 bp, Homo sapiens
atgggcaaaggagatcctaagaagccgacaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcttccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatatagctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagaagatgaagaagaagatgatgatgatgaa

SEQ ID NO: 10 - protein, 216 aa, Homo sapiens [sequence omitted]

SEQ ID NO: 11 - DNA, 633 bp, Homo sapiens
atgggcaaaggagatcctaagaagccgagaggcaaaatgtcatcatatgcattttttgtgcaaacttgtcgggaggagcataagaagaagcactcagatgcttcagtcaacttctcagagttttctaacaagtgctcagagaggtggaagaccatgtctgctaaagagaaaggaaaatttgaggatatggcaaaggcggacaagacccattatgaaagacaaatgaaaacctatatccctcccaaaggggagacaaaaaagaagttcaaggatcccaatgcacccaagaggcctccttcggccttcttcctgttctgctctgagtatcacccaaaaatcaaaggagaacatcctggcctgtccattggtgatgttgcgaagaaactgggagagatgtggaataacactgctgcagatgacaagcagcctggtgaaaagaaggctgcgaagctgaaggaaaaatacgaaaaggatattgctgcatatcaagctaaaggaaagcctgaggcagcaaaaaagggagttgtcaaagctgaaaaaagcaagaaaaagaaggaagaggaggaagatgaggaagatgaagaggatgaggaggaggaagatgaagaagatgaagaagatgatgatgatgaa

SEQ ID NO: 12 - protein, 211 aa, Homo sapiens
MGKGDPKKPRGKMSSYAFFVQTCREEHKKKHSDASVNFSEFSNKCSERWKTMSAKEKGKFEDMAKADKTHYERQMKTYIPPKGETKKKFKDPNAPKRPPSAFFLFCSEYHPKIKGEHPGLSIGDVAKKLGEMWNNTAADDKQPGEKKAAKLKEKYEKDIAAYQAKGKPEAAKKGVVKAEKSKKKKEEEEDEEDEEDEEEEDEEDEEDDDDE

SEQ ID NO: 13 - DNA, 564 bp, Homo sapiens
atgggcaaaggagaccctaagaagccgagaggcaaaatgtcatcatatgcattttttgtgcaaacttgtcgggaggagtgtaagaagaagcacccagatgcttcagtcaacttctcagagttttctaagaagtgctcagagaggtggaaggccatgtctgctaaagataaaggaaaatttgaagatatggcaaaggtggacaaagaccgttatgaaagagaaatgaaaacctatatccctcctaaaggggagacaaaaaagaagttcgaggattccaatgcacccaagaggcctccttcggcctttttgctgttctgctctgagtattgcccaaaaatcaaaggagagcatcctggcctgcctattagcgatgttgcaaagaaactggtagagatgtggaataacacttttgcagatgacaagcagctttgtgaaaagaaggctgcaaagctgaaggaaaaatacaaaaaggatacagctacatatcgagctaaaggaaagcctgatgcagcaaaaaagggagttgtcaaggctgaaaaaagcaagaaaaagaaggaagaggag

SEQ ID NO: 14 - protein, 188 aa, Homo sapiens
MGKGDPKKPRGKMSSYAFFVQTCREECKKKHPDASVNFSEFSKKCSERWKAMSAKDKGKFEDMAKVDKDRYEREMKTYIPPKGETKKKFEDSNAPKRPPSAFLLFCSEYCPKIKGEHPGLPISDVAKKLVEMWNNTFADDKQLCEKKAAKLKEKYKKDTATYRAKGKPDAAKKGVVKAEKSKKKKEEE

SEQ ID NO: 15 - DNA, 615 bp, Homo sapiens
atggacaaagcagatcctaagaagctgagaggtgaaatgttatcatatgcattttttgtgcaaacttgtcaggaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaa

SEQ ID NO: 16 - protein, 205 aa, Homo sapiens [sequence omitted]

SEQ ID NO: 17 - DNA, 240 bp, Homo sapiens
atgggcaaaggagatcctaagaagccgagaggcaaaatgtcatcatgtgcattttttgtgcaaacttgttgggaggagcataagaagcagtacccagatgcttcaatcaacttctcagagttttctcagaagtgcccagagacgtggaagaccacgattgctaaagagaaaggaaaatttgaagatatgccaaaggcagacaaggcccattatgaaagagaaatgaaaacctatataccc

SEQ ID NO: 18 - protein, 80 aa, Homo sapiens
MGKGDPKKPRGKMSSCAFFVQTCWEEHKKQYPDASINFSEFSQKCPETWKTTIAKEKGKFEDMPKADKAHYEREMKTYIP

SEQ ID NO: 19 - DNA, 240 bp, Homo sapiens
aaacagagaggcaaaatgccatcgtatgtattttgtgtgcaaacttgtccggaggagcgtaagaagaaacacccagatgcttcagtcaacttctcagagttttctaagaagtgcttagtgagggggaagaccatgtctgctaaagagaaaggacaatttgaagctatggcaagggcagacaaggcccgttacgaaagagaaatgaaaacatatatccctcctaaaggggagacaaaaaaa

SEQ ID NO: 20 - protein, 80 aa, Homo sapiens
KQRGKMPSYVFCVQTCPEERKKKHPDASVNFSEFSKKCLVRGKTMSAKEKGQFEAMARADKARYEREMKTYIPPKGETKK

SEQ ID NO: 21 - DNA, 258 bp, Homo sapiens
atgggcaaaagagaccctaagcagccaagaggcaaaatgtcatcatatgcattttttgtgcaaactgctcaggaggagcacaagaagaaacaactagatgcttcagtcagtttctcagagttttctaagaactgctcagagaggtggaagaccatgtctgttaaagagaaaggaaaatttgaagacatggcaaaggcagacaaggcctgttatgaaagagaaatgaaaatatatccctacttaaaggggagacaaaaa

SEQ ID NO: 22 - protein, 86 aa, Homo sapiens
MGKRDPKQPRGKMSSYAFFVQTAQEEHKKKQLDASVSFSEFSKNCSERWKTMSVKEKGKFEDMAKADKACYEREMKIYPYLKGRQK

SEQ ID NO: 23 - DNA, 211 bp, Homo sapiens
atgggcaaaggagaccctaagaagccaagagagaaaatgccatcatatgcattttttgtgcaaacttgtagggaggcacataagaacaaacatccagatgcttcagtcaactcctcagagttttctaagaagtgctcagagaggtggaagaccatgcctactaaacagaaaggaaaattcgaagatatggcaaaggcagacagggcccata

SEQ ID NO: 24 - protein, 70 aa, Homo sapiens
MGKGDPKKPREKMPSYAFFVQTCREAHKNKHPDASVNSSEFSKKCSERWKTMPTKQKGKFEDMAKADRAH

SEQ ID NO: 25 - protein, 54 aa, Homo sapiens; identical to SEQ ID NO: 4

SEQ ID NO: 26 - protein, 53 aa, Homo sapiens
DSSVNFAEFSKKCSERWKTMSAKEKSKFEDMAKSDKARYDREMKNYVPPKGDK

SEQ ID NO: 27 - protein, 54 aa, Homo sapiens
PEVPVNFAEFSKKCSERWKTVSGKEKSKFDEMAKADKVRYDREMKDYGPAKGGK

SEQ ID NO: 28 - protein, 54 aa, Homo sapiens; identical to SEQ ID NO: 4

SEQ ID NO: 29 - protein, 54 aa, Homo sapiens
SDASVNFSEFSNKCSERWKTMSAKEKGKFEDMAKADKTHYERQMKTYIPPKGET

SEQ ID NO: 30 - protein, 54 aa, Homo sapiens
PDASVNFSEFSKKCSERWKAMSAKDKGKFEDMAKVDKADYEREMKTYIPPKGET

SEQ ID NO: 31 - protein, 54 aa, Homo sapiens
PDASVKFSEFLKKCSETWKTIFAKEKGKFEDMAKADKAHYEREMKTYIPPKGEK

SEQ ID NO: 32 - protein, 54 aa, Homo sapiens
PDASINFSEFSQKCPETWKTTIAKEKGKFEDMAKADKAHYEREMKTYIPPKGET

SEQ ID NO: 33 - protein, 38 aa, Homo sapiens
PDASVNSSEFSKKCSERWKTMPTKQGKFEDMAKADRAH

SEQ ID NO: 34 - protein, 54 aa, Homo sapiens
PDASVNFSEFSKKCLVRGKTMSAKEKGQFEAMARADKARYEREMKTYIPPKGET

SEQ ID NO: 35 - protein, 54 aa, Homo sapiens
LDASVSFSEFSNKCSERWKTMSVKEKGKFEDMAKADKACYEREMKIYPYLKGRQ

SEQ ID NO: 36 - protein, 84 aa, Homo sapiens
GKGDPKKPRGKMSSYAFFVQTCREEHKKKHPDASVNFSEFSKKCSERWKTMSAKEKGKFEDMAKADKARYEREMKTYIPPKGET

SEQ ID NO: 37 - protein, 74 aa, Homo sapiens
FKDPNAPKRPPSAFFLFCSEYRPKIKGEHPGLSIGDVAKKLGEMWNNTAADDKQPYEKKAAKLKEKYEKDIAAY

SEQ ID NO: 38 - protein, 74 aa, Homo sapiens
KKDPNAPKRPPSAFFLFCSEHRPKIKSEHPGLSIGDTAKKLGEMWSEQSAKDKQPYEQKAAKLKEKYEKDIAAY

SEQ ID NO: 39 - protein, 74 aa, Homo sapiens; identical to SEQ ID NO: 8

SEQ ID NO: 40 - protein, 74 aa, Homo sapiens
FKDPNAPKRPPSAFFLFCSEYHPKIKGEHPGLSIGDVAKKLGEMWNNTAADDKQPGEKKAAKLKEKYEKDIAAY

SEQ ID NO: 41 - protein, 74 aa, Homo sapiens
FKDSNAPKRPPSAFLLFCSEYCPKIKGEHPGLPISDVAKKLVEMWNNTFADDKQLCEKKAAKLKEKYKKDTATY

SEQ ID NO: 42 - protein, 74 aa, Homo sapiens
FKDPNAPKRPPSAFFLFCSEYRPKIKGEHPGLSIGDVVKKLAGMWNNTAAADKQFYEKKAAKLKEKYKKDIAAY

SEQ ID NO: 43 - protein, 92 aa, Homo sapiens
FKDPNAPKRPPSAFFLFCSEYRPKIKGEHPGLSIGDVAKKLGEMWNNTAADDKQPYEKKAAKLKEKYEKDIAAYRAKGKPDAAKKGVVKAEK

SEQ ID NO: 44 - protein, 20 aa, Homo sapiens
NAPKRPPSAFFLFCSEYRPK

SEQ ID NO: 45 - protein, 20 aa, Homo sapiens
FKDPNAPKRLPSAFFLFCSE

SEQ ID NO: 46 - protein, 6 aa, Artificial Sequence
  Artificially synthesized Caspase inhibitor peptide
IQACRG

SEQ ID NO: 47 - protein, 4 aa, Artificial Sequence
  Artificially synthesized Caspase inhibitor peptide
WEHD

SEQ ID NO: 48 - protein, 4 aa, Artificial Sequence
  Artificially synthesized Caspase inhibitor peptide
YVAD

SEQ ID NO: 49 - protein, 4 aa, Artificial Sequence
  Artificially synthesized Caspase inhibitor peptide
IETD

SEQ ID NO: 50 - protein, 4 aa, Artificial Sequence
  Artificially synthesized Caspase inhibitor peptide
AEVD

SEQ ID NO: 51 - protein, 4 aa, Artificial Sequence
  Artificially synthesized Caspase inhibitor peptide
LEED

SEQ ID NO: 52 - protein, 4 aa, Artificial Sequence
  Artificially synthesized Caspase inhibitor peptide
LEVD

SEQ ID NO: 53 - protein, 4 aa, Artificial Sequence
  Artificially synthesized Caspase inhibitor peptide
YVKD

SEQ ID NO: 54 - protein, 5 aa, Artificial Sequence
  Artificially synthesized Caspase inhibitor peptide
VDVAD

SEQ ID NO: 55 - protein, 5 aa, Artificial Sequence
  Artificially synthesized Caspase inhibitor peptide
LDESD

SEQ ID NO: 56 - protein, 4 aa, Artificial Sequence
  Artificially synthesized Caspase inhibitor peptide
DEVD

SEQ ID NO: 57 - protein, 4 aa, Artificial Sequence
  Artificially synthesized Caspase inhibitor peptide
DMQD

SEQ ID NO: 58 - protein, 4 aa, Artificial Sequence
  Artificially synthesized Caspase inhibitor peptide
DQTD

SEQ ID NO: 59 - protein, 4 aa, Artificial Sequence
  Artificially synthesized Caspase inhibitor peptide
VEID

SEQ ID NO: 60 - protein, 4 aa, Artificial Sequence
  Artificially synthesized Caspase inhibitor peptide
IETD

SEQ ID NO: 61 - protein, 4 aa, Artificial Sequence
  Artificially synthesized Caspase inhibitor peptide
LEHD

SEQ ID NO: 62 - protein, 21 aa, Artificial Sequence
  Artificially synthesized Caspase inhibitor peptide
AAVALLPAVLLALLALPIETD

SEQ ID NO: 63 - protein, 5 aa, Artificial Sequence
  Artificially synthesized Caspase inhibitor peptide
AYVHD

(19) United States
(12) Patent Application Publication
(10) Pub. No.: US 2010/0040607 A1
(43) Pub. Date: Feb. 18, 2010

(54) Combination Therapy with Inhibitors of HMGB and Caspase for the Treatment of Inflammatory Diseases

(76) Inventors: Kevin J. Tracey, Old Greenwich, CT (US); Huan Yang, Douglaston, NY (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/920,296

(22) PCT Filed: May 11, 2006
(86) PCT No.: PCT/US2006/018152

§ 371 Date: Jan. 31, 2008

Related U.S. Application Data
(60) Provisional application No. 60/680,975, filed on May 13, 2005.

(51) Int. Cl. A61K 39/395; A61K 38/16; A61K 31/19; A61K 31/215; A61K 38/05; A61K 31/7088; A61K 38/06; A61K 38/07; A61K 38/08; A61K 38/10; A61K 38/48; A61K 31/50; A61P 29/00

(52) U.S. Cl. 424/133.1; 514/2; 424/130.1; 424/141.1; 514/557; 514/529; 514/12; 514/16; 514/15; 424/94.63; 514/18; 514/17; 514/19; 514/247; 514/44 R

(57) ABSTRACT

Compositions and methods are disclosed for treating a condition characterized by activation of an inflammatory cytokine cascade in a patient. The compositions comprise an agent that inhibits HMGB biological activity and a caspase inhibitor. The methods comprise treating a cell or a patient with sufficient amounts of the composition to inhibit the release of proinflammatory cytokine(s) and/or inhibit the inflammatory cytokine cascade.

### RELATED APPLICATION

This application claims the benefit of U.S. Provisional Application No. 60/680,975, filed on May 13, 2005. The entire teachings of the above application are incorporated herein by reference.

### GOVERNMENT SUPPORT

The invention was supported, in whole or in part, by a grant from NIGMS (Grant #GM62508). The Government has certain rights in the invention.

### BACKGROUND

Inflammation is often induced by proinflammatory cytokines, such as tumor necrosis factor (TNF), interleukin (IL)-1α, IL-1β, IL-6, macrophage migration inhibitory factor (MIF), and other compounds. These proinflammatory cytokines are produced by several different cell types, most importantly immune cells (for example, monocytes, macrophages and neutrophils), but also non-immune cells such as fibroblasts, osteoblasts, smooth muscle cells, epithelial cells, and neurons. These proinflammatory cytokines contribute to various disorders during the early stages of an inflammatory cytokine cascade.

High mobility group box chromosomal protein 1 (HMGB1) is an intranuclear protein, which binds DNA and is involved in the organization of chromatin (Bustin, M., Mol. Cell. Biol. 19:5237-46 (1999)). The HMGB1 protein has three domains: two DNA binding motifs termed HMGB A and HMGB B boxes, and an acidic carboxyl terminus. The two HMGB boxes are highly conserved 80 amino acid, L-shaped domains.

HMGB1 acts as a pro-inflammatory cytokine (Yang H., et al., Shock 15:247-53 (2001)), and can be actively secreted by macrophages/monocytes by inflammatory stimuli (Wang H., et al., Science 285:248-51 (1999)). During secretion, HMGB1 exits the nucleus and is transported through the cytoplasm, where it is actively released to the extracellular space. HMGB1 can also be passively released from the nuclei of necrotic or damaged cells (Scaffidi P., et al., Nature 418:191-95 (2002)). Both TNF-α and IL-1β have been shown to stimulate the release of HMGB1 (Wang H., et al., Surgery 126:389-92 (1999)), and HMGB1 may in turn stimulate the synthesis of pro-inflammatory cytokines (Andersson, U., et al., J. Exp. Med. 192:565-570 (2000)).

A family of cysteine protease enzymes, known as caspases, are involved in pro-inflammatory cytokine processing and play an important role in inflammation (Thornberry, N. A., Chem. Biol. 5, R97-R103 (1998)). Caspase-1, the most studied enzyme of this class, activates IL-1β via proteolytic cleavage of its pre-cursor form and plays a key role in the inflammatory response. Other caspases are of central importance in the intracellular signaling pathways leading to apoptosis.

A wide range of caspase inhibitors have been developed. The majority of such inhibitors target a specific subset of caspases. For example, caspases recognize particular tetrapeptide sequences on their substrates and, as a consequence, these sequences have been utilized in designing specific peptide inhibitors. Several peptide compounds, which mimic caspase substrate recognition motifs, have been used successfully as specific caspase inhibitors (Garcia-Calvo M., et al., J Biol. Chem. 273(49):32608-32613 (1998)). In addition, a variety of small molecule caspase inhibitors, which possess improved pharmacological properties relative to peptide inhibitors, have been developed (see, e.g., U.S. Pat. No. 6,566,338 B1).

HMGB1 has been implicated as a cytokine mediator of a number of inflammatory conditions. In addition, fragments of HMGB modulate inflammation. The delayed kinetics of HMGB1 appearance during endotoxemia make it a potentially good therapeutic target in the treatment of inflammatory conditions. However, what is needed are improved therapies for treating inflammatory conditions.

### SUMMARY

The present invention is based on the discovery that combination therapies involving agents that inhibit HMGB biological activity and agents that inhibit caspase activity can be used for the treatment of inflammatory conditions. Agents that inhibit HMGB biological activity include, e.g., HMGB A boxes, antibodies to HMGB (e.g., antibodies to HMGB B boxes, antibodies to HMGB A boxes), HMGB small molecule inhibitors, cholinergic agonists (e.g., α7 nicotinic agonists), antibodies to TLR2, soluble TLR2 polypeptides, antibodies to RAGE, soluble RAGE polypeptides and RAGE small molecule antagonists. Agents that inhibit caspase activity include, e.g., peptide and modified peptide compounds, small molecule caspase inhibitors, inhibitors of apoptosis (IAP) proteins, FLICE inhibitory proteins (FLIP), dominant negative caspases and antisense or small double-stranded interfering RNA (RNA interference (RNAi)) that targets one or more caspases.

Accordingly, in one embodiment, the invention is a pharmaceutical composition comprising an agent that inhibits HMGB biological activity and a caspase inhibitor. In one embodiment, the agent that inhibits HMGB biological activity inhibits one or more activities selected from the group consisting of: inhibiting HMGB receptor binding; inhibiting HMGB signaling; inhibiting HMGB-mediated inflammation; inhibiting HMGB-mediated release of a proinflammatory cytokine; inhibiting HMGB-mediated activation of antigen presenting cells; and inhibiting HMGB-induced smooth muscle cell migration. In another embodiment, the agent that inhibits HMGB biological activity is selected from the group consisting of: an HMGB A box or a biologically active fragment thereof; an antibody that binds to an HMGB polypeptide or an antigen-binding fragment thereof; an HMGB small molecule inhibitor; an antibody to TLR2 or an antigen-binding fragment thereof; a soluble TLR2 polypeptide; an antibody to RAGE or an antigen-binding fragment thereof; a soluble RAGE polypeptide and a RAGE small molecule antagonist.

In another embodiment, the agent that inhibits caspase activity is selected from the group consisting of a peptide or modified peptide compound, a small molecule caspase inhibitor, an inhibitor of apoptosis (IAP) protein, a FLICE inhibitory protein (FLIP), a dominant negative caspase and an antisense or small double-stranded interfering RNA (RNA interference (RNAi)) that targets one or more caspases.

In another embodiment, the invention is a method of treating an inflammatory condition in a patient comprising administering to the patient a composition comprising an agent that inhibits HMGB biological activity and an agent that inhibits caspase activity.

The present invention provides new combination therapy compositions and methods of treatment for patients with inflammatory conditions.

### BRIEF DESCRIPTION OF THE DRAWINGS

FIG. 1A is the amino acid sequence of a human HMG1 polypeptide (SEQ ID NO:1).

FIG. 1B is the amino acid sequence of a rat and mouse HMG1 polypeptide (SEQ ID NO:2).

FIG. 1C is the amino acid sequence of a human HMG2 polypeptide (SEQ ID NO:3).

FIG. 1D is the amino acid sequence of a human, mouse, and rat HMG1 A box polypeptide (SEQ ID NO:4).

FIG. 1E is the amino acid sequence of a human, mouse, and rat HMG1 B box polypeptide (SEQ ID NO:5).

FIG. 2A is the nucleic acid sequence of HMG1L5 (formerly HMG1L10; SEQ ID NO:9), which encodes an HMGB polypeptide.

FIG. 2B is the polypeptide sequence of HMG1L5 (formerly HMG1L10; SEQ ID NO:10).

FIG. 2C is the nucleic acid sequence of HMG1L1 (SEQ ID NO:11), which encodes an HMGB polypeptide.

FIG. 2D is the polypeptide sequence of HMG1L1 (SEQ ID NO:12).

FIG. 2E is the nucleic acid sequence of HMG1L4 (SEQ ID NO:13), which encodes an HMGB polypeptide.

FIG. 2F is the polypeptide sequence of HMG1L4 (SEQ ID NO:14).

FIG. 2G is the nucleic acid sequence of the BAC clone RP11-395A23 (SEQ ID NO:15), which encodes an HMG polypeptide sequence.

FIG. 2H is the amino acid sequence of the BAC clone RP11-395A23 (SEQ ID NO:16).

FIG. 2I is the nucleic acid sequence of HMG1L9 (SEQ ID NO:17), which encodes an HMGB polypeptide.

FIG. 2J is the polypeptide sequence of HMG1L9 (SEQ ID NO:18).

FIG. 2K is the nucleic acid sequence of LOC122441 (SEQ ID NO:19), which encodes an HMGB polypeptide.

FIG. 2L is the polypeptide sequence of LOC122441 (SEQ ID NO:20).

FIG. 2M is the nucleic acid sequence of LOC139603 (SEQ ID NO:21), which encodes an HMGB polypeptide.

FIG. 2N is the polypeptide sequence of LOC139603 (SEQ ID NO:22).

FIG. 2O is the nucleic acid sequence of HMG1L8 (SEQ ID NO:23), which encodes an HMGB polypeptide.

FIG. 2P is the polypeptide sequence of HMG1L8 (SEQ ID NO:24).

FIGS. 3A-I depict a series of sections of the spleen subjected to TUNEL, Caspase 3 and Annexin VI staining. Specifically, either normal mice (“Normal”) or mice that had undergone cecal ligation and puncture (CLP) and received either a single dose of mAb 6E6 or control IgG antibody (10 μg/mouse, IP) at 24 hours after surgery and were sacrificed at 40 hours after CLP.

FIG. 3A is a fluorescent image showing TUNEL staining in a fixed section of spleen from a normal mouse. Apoptotic cells stain positively for this marker. Image is shown at 100× magnification.

FIG. 3B is a fluorescent image showing Caspase 3 immunofluorescence in a fixed section of spleen from a normal mouse. Apoptotic cells stain positively for this marker. Image is shown at 100× magnification.

FIG. 3C is a fluorescent image showing-Annexin VI immunofluorescence in a fixed section of spleen from a normal mouse. Apoptotic cells stain positively for this marker. Image is shown at 100× magnification.

FIG. 3D is a fluorescent image showing TUNEL staining in a fixed section of spleen from a septic mouse, following cecal ligation and puncture (CLP) surgery. The mouse received a dose of control IgG (IgG) 24 hours after surgery. Apoptotic cells stain positively for this marker. Image is shown at 100× magnification.

FIG. 3E is a fluorescent image showing Caspase 3 immunofluorescence in a fixed section of spleen from a septic mouse, following cecal ligation and puncture (CLP) surgery. The mouse received a dose of control IgG (IgG) 24 hours after surgery. Apoptotic cells stain positively for this marker. Image is shown at 100× magnification.

FIG. 3F is a fluorescent image showing Annexin VI immunofluorescence in a fixed section of spleen from a septic mouse, following cecal ligation and puncture (CLP) surgery. The mouse received a dose of control IgG (IgG) 24 hours after surgery. Apoptotic cells stain positively for this marker. Image is shown at 100× magnification. Arrows indicate cells that stained positively for Annexin VI.

FIG. 3G is a fluorescent image showing TUNEL staining in a fixed section of spleen from a septic mouse, following cecal ligation and puncture (CLP) surgery. The mouse received a dose of monoclonal antibody 6E6 (mAb) 24 hours after surgery. Apoptotic cells stain positively for this marker. Image is shown at 100× magnification.

FIG. 3H is a fluorescent image showing Caspase 3 immunofluorescence in a fixed section of spleen from a septic mouse, following cecal ligation and puncture (CLP) surgery. The mouse received a dose of monoclonal antibody 6E6 (mAb) 24 hours after surgery. Apoptotic cells stain positively for this marker. Image is shown at 100× magnification.

FIG. 3I is a fluorescent image showing Annexin VI immunofluorescence in a fixed section of spleen from a septic mouse, following cecal ligation and puncture (CLP) surgery. The mouse received a dose of monoclonal antibody 6E6 (mAb) 24 hours after surgery. Apoptotic cells stain positively for this marker. Image is shown at 100× magnification. Arrows indicate cells that stained positively for Annexin VI.

FIG. 4 is a Western blot depicting HMGB1 protein levels in RAW 264.7 cells after treatment with lipopolysaccharide (LPS) alone, or LPS with various concentrations of the caspase inhibitor, Z-VAD-FMK (VAD), or the control peptide, Z-FA-FMK (Ctrl).

FIG. 5A is a confocal fluorescent image of HMGB antibody staining in normal, untreated RAW 264.7 cells.

FIG. 5B is a confocal fluorescent image of HMGB1 antibody staining in RAW 264.7 cells after treatment with LPS.

FIG. 5C is a confocal fluorescent image of HMGB1 antibody staining in RAW 264.7 cells after treatment with LPS and the caspase inhibitor, Z-VAD-FMK (VAD).

FIG. 5D is a confocal fluorescent image of HMGB1 antibody staining in RAW 264.7 cells after treatment with LPS and the control peptide, Z-FA-FMK (Ctrl).

FIG. 6A is a bar graph depicting the concentration of HMGB1 protein in bronchoalveolar fluid (BAL) of sacrificed mice that were either untreated Normal) or subjected to cecal ligation and puncture (CLP) to induce sepsis. Following CLP surgery, the mice were injected intraperitoneally with either the caspase inhibitor, Z-VAD-FMK (labeled as “CLP+VAD”), or the control peptide, Z-FA-FMK (labeled as “CLP”). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 6B is a bar graph depicting the concentration of HMGB1 protein in peritoneal lavage fluid (Peritoneal) of sacrificed mice that were either untreated (Normal) or subjected to cecal ligation and puncture (CLP) to induce sepsis. Following CLP surgery, the mice were injected intraperitoneally with either the caspase inhibitor, Z-VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 6C is a bar graph depicting the concentration of HMGB1 protein in serum of sacrificed mice that were either untreated (Normal) or subjected to cecal ligation and puncture (CLP) to induce sepsis. Following CLP surgery, the mice were injected intraperitoneally with either the caspase inhibitor, Z-VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 7A is a bar graph depicting the concentration of the cytokine, IL-6, in the serum of sacrificed BALB/c mice that were either untreated (Normal) or subjected to cecal ligation and puncture (CLP) to induce sepsis. Following surgery, the CLP mice were injected intraperitoneally with either the caspase inhibitor, Z VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 7B is a bar graph depicting the concentration of the cytokine, KC, in the serum of sacrificed BALB/c mice that were either untreated (Normal) or subjected to cecal ligation and puncture (CLP) to induce sepsis. Following surgery, the CLP mice were injected intraperitoneally with either the caspase inhibitor, Z-VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 7C is a bar graph depicting the concentration of the cytokine, MIP-2, in the serum of sacrificed BALB/c mice that were either untreated (Normal) or subjected to cecal ligation and puncture (CLP) to induce sepsis. Following CLP surgery, the mice were injected intraperitoneally with either the caspase inhibitor, Z-VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 7D is a bar graph depicting the concentration of the cytokine, KC, in the BAL of sacrificed BALB/c mice that were either untreated (Normal) or subjected to cecal ligation and puncture (CLP) to induce sepsis. Following surgery, the CLP mice were injected intraperitoneally with either the caspase inhibitor, Z-VAD-FMK (CLP+VAD), or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 7E is a bar graph depicting the concentration of the cytokine, MIP-2, in the BAL of sacrificed BALB/c mice that were either untreated (Normal) or subjected to cecal ligation and puncture (CLP) to induce sepsis. Following surgery, the CLP mice were injected intraperitoneally with either the caspase inhibitor, Z-VAD-FMK (CLP+VAD) or the control peptide, Z-FA-FMK (CLP). The asterisk above the CLP+VAD bar indicates that the increase in HMGB1 protein level in the fluid, relative to the level of HMGB1 protein in the fluid of CLP control mice, is statistically significant (P<0.05), as determined by unpaired t-test. N=7 mice per group.

FIG. 8A depicts Caspase 3 antibody staining in cells from sections of spleen from sacrificed BALB/c mice that were untreated (Normal).

FIG. 8B depicts Caspase 3 antibody staining in cells from sections of spleen from sacrificed BALB/c mice that were subjected to cecal ligation and puncture followed by injection of Z-FA-FMK control peptide (CLP+ctrl).

FIG. 8C depicts Caspase 3 antibody staining in cells from sections of spleen from sacrificed BALB/c mice that were subjected to cecal ligation and puncture followed by injection of Z-VAD-FMK caspase inhibitor (CLP+VAD).

FIG. 8D depicts Caspase 3 antibody staining in cells from sections of thymus from sacrificed BALB/c mice that were untreated (Normal).

FIG. 8E depicts Caspase 3 antibody staining in cells from sections of thymus from sacrificed BALB/c mice that were subjected to cecal ligation and puncture followed by injection of Z-FA-FMK control peptide (CLP+ctrl).

FIG. 8F depicts Caspase 3 antibody staining in cells from sections of thymus from sacrificed BALB/c mice that were subjected to cecal ligation and puncture followed by injection of Z-VAD-FMK caspase inhibitor (CLP+VAD).

FIG. 8G depicts TUNEL staining in cells from sections of spleen from sacrificed BALB/c mice that were untreated (Normal).

FIG. 8H depicts TUNEL staining in cells from sections of spleen from sacrificed BALB/c mice that were subjected to cecal ligation and puncture followed by injection of Z-FA-FMK control peptide (CLP+ctrl).

FIG. 8I depicts TUNEL staining in cells from sections of spleen from sacrificed BALB/c mice that were subjected to cecal ligation and puncture followed by injection of Z-VAD-FMK caspase inhibitor (CLP+VAD).

FIG. 8J depicts TUNEL staining in cells from sections of thymus from sacrificed BALB/c mice that were untreated (Normal).

FIG. 8K depicts TUNEL staining in cells from sections of thymus from sacrificed BALB/c mice that were subjected to cecal ligation and puncture followed by injection of Z-FA-FMK control peptide (CLP+ctrl).

FIG. 8L depicts TUNEL staining in cells from sections of thymus from sacrificed BALB/c mice that were subjected to cecal ligation and puncture followed by injection of Z-VAD-FMK caspase inhibitor (CLP+VAD).

FIG. 9A is a graph depicting the concentration of MIP-2 released from RAW 264.7 cells, which were incubated for 16 hours with either untreated RAW 246.7 cells (Normal cells), RAW 246.7 cells that were treated with 6 μM Camptothein to induce apoptosis (Apoptotic cells) or RAW 246.7 cells that were subjected to repeated freeze/thaw cycles to induce necrosis (Necrotic cells).

FIG. 9B is a graph depicting concentration of TNF released from RAW 264.7 cells, which were incubated for 16 hours with either untreated RAW 246.7 cells (Normal cells), RAW 246.7 cells that were treated with 6 μM Camptothein to induce apoptosis (Apoptotic cells) or RAW 246.7 cells that were subjected to repeated freeze/thaw cycles to induce necrosis (Necrotic cells).

FIG. 10A is a Western blot depicting levels of HMGB1 protein released from RAW 264.7 cells that were incubated for 16 hours with either untreated RAW 246.7 cells (Normal cells) or RAW 246.7 cells that were treated with 6 μM Camptothein to induce apoptosis (Apoptotic cells).

FIG. 10B is a Western blot depicting levels of HMGB1 protein released from RAW 264.7 cells that were subjected to repeated freeze/thaw cycles to induce necrosis (Necrotic cells).

### DETAILED DESCRIPTION

Compositions Comprising an Agent that Inhibits HMGB Biological Activity and a Caspase Inhibitor (Combination Therapy Compositions)

The present invention is based on the discovery that caspase inhibitors can be combined with agents that inhibit HMGB biological activity to form pharmaceutical compositions (combination therapy compositions) for use in treating an inflammatory condition in a patient. Such compositions are collectively termed “combination therapy compositions”. As described herein, agents that inhibit HMGB biological activity include, e.g., agents that inhibit HMGB receptor binding, agents that inhibit HMGB signaling, agents that inhibit HMGB-mediated inflammation, agents that inhibit HMGB-mediated release of a proinflammatory cytokine, agents that inhibit HMGB-mediated activation of antigen presenting cells (WO 03/026691) and agents that inhibit HMGB-mediated induction of smooth muscle cell migration (WO 02/074337). Such agents that inhibit HMGB biological activity encompass agents that interact directly with HMGB and inhibit HMGB biological activity (e.g., antibodies to HMGB), as well as agents that do not interact directly with HMGB and inhibit HMGB biological activity (e.g., antibodies to TLR2, soluble TLR2 polypeptides, antibodies to RAGE, soluble RAGE polypeptides, RAGE small molecule antagonists). As described herein, agents that inhibit caspase activity include, e.g., peptide and modified peptide compounds, small molecule caspase inhibitors, inhibitors of apoptosis (IAP) proteins, FLICE inhibitory proteins (FLIP), dominant negative caspases and antisense or small double-stranded interfering RNA (RNA interference (RNAi)) that target one or more caspases.

In one embodiment, the invention is a pharmaceutical composition comprising an agent that inhibits HMGB biological activity and a caspase inhibitor. In another embodiment, the agent that inhibits HMGB biological activity inhibits one or more activities selected from the group consisting of: inhibiting HMGB receptor binding; inhibiting HMGB signaling; inhibiting HMGB-mediated inflammation; inhibiting HMGB-mediated release of a proinflammatory cytokine; inhibiting HMGB-mediated activation of antigen presenting cells; and inhibiting HMGB-mediated induction of smooth muscle cell migration. In still another embodiment, the agent that inhibits HMGB biological activity binds to HMGB. In yet another embodiment, the agent does not bind to HMGB and inhibits HMGB biological activity.

In one embodiment, the agent that inhibits HMGB biological activity is selected from the group consisting of: an HMGB A box or a biologically active fragment thereof; an antibody that binds to an HMGB polypeptide or an antigen-binding fragment thereof; an HMGB small molecule inhibitor; a cholinergic agonist (e.g., an α7 nicotinic receptor agonist), an antibody to TLR2 or an antigen-binding fragment thereof; a soluble TLR2 polypeptide; an antibody to RAGE or an antigen-binding fragment thereof; a soluble RAGE polypeptide and a RAGE small molecule antagonist.

A proinflammatory domain of HMGB (e.g., HMGB1) is the B box, and antibodies that bind to the B box and inhibit proinflammatory cytokine release and inflammatory cytokine cascades can be used to alleviate deleterious symptoms caused by inflammatory cytokine cascades (PCT Publication No. WO 02/092004, the entire teachings of which are incorporated herein by reference). In addition to antibodies that bind to the B box of HMGB and inhibit proinflammatory cytokine release, antibodies that bind to the A box of HMGB can also inhibit proinflammatory cytokine release and are useful in the methods of the invention.

The A box of HMGB (e.g., HMGB1) is a weak agonist of inflammatory cytokine release, and competitively inhibits the proinflammatory activity of the B box and of HMGB (e.g., HMGB1) (CT Publication No. WO 02/092004). Thus, HMGB A boxes (e.g., the A box of HMGB1) can be used as HMGB antagonists in the combination therapy compositions and methods of the invention.

Other HMGB inhibitors (e.g., inhibitors of HMGB receptor binding and/or HMGB signaling) include, e.g., antibodies to RAGE or antigen-binding fragments thereof (e.g., as taught in U.S. Pat. Nos. 5,864,018 and 5,852,174), antibodies to TLR2 or antigen-binding fragments thereof (e.g., as taught in PCT Publication Nos. WO 01/36488 and WO 00/75358), soluble RAGE, soluble TLR2 (e.g., as taught in Iwaki et al., J. Biol. Chem. 277(27):24315-24320 (2002)), HMGB small molecule inhibitors (e.g., ethyl pyruvate (as described in US 20040110833; the teachings of which are incorporated herein by reference in their entirety), certain derivatives of isoxazole, isoxazolidine, isothiazole and isothiazolidine compounds (as described in WO 2005034952; the teachings of which are incorporated herein by reference in their entirety), RAGE small molecule antagonists (e.g., as taught in PCT Publication Nos. WO 01/99210, WO 02/06965 and WO 03/075921, and U.S. Published Application No. 2002/0193432A1), TLR2 small molecule antagonists, TLR2 dominant mutant proteins, and RAGE dominant mutant proteins. Such HMGB inhibitors can be combined with a caspase inhibitor to treat inflammatory conditions.

### HMGB Polypeptides

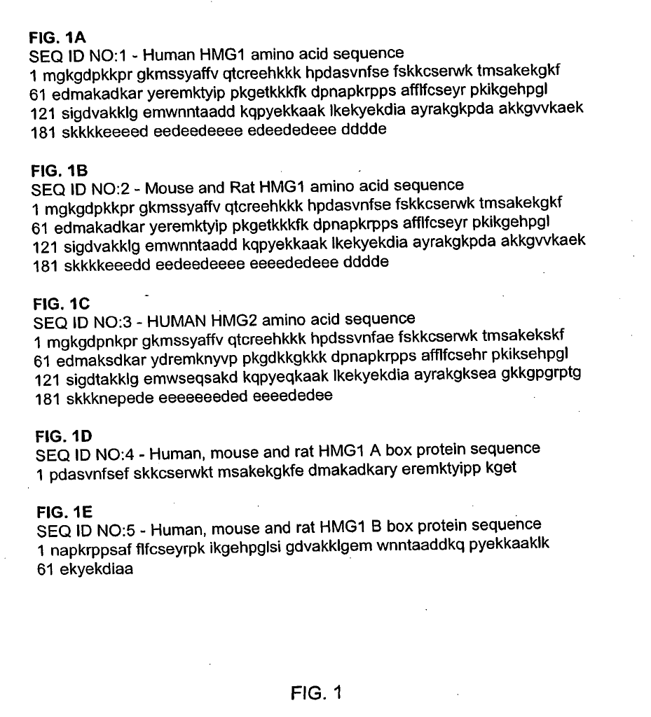

As used herein, an “HMGB polypeptide” is polypeptide that has at least 60%, more preferably, at least 70%, 75%, 80%, 85%, or 90%, and most preferably at least 95% sequence identity to a sequence selected from the group consisting of SEQ ID NO:1 (FIG. 1A), SEQ ID NO:2 (FIG. 1B), SEQ ID NO:3 (FIG. 1C), and SEQ ID NO: 6 (MGKGDPKKPTGKMSSYAFFVQTCREEHKKKHPDASVNFSEFSKKC SERWKTMSAKEKGKFEDMAKADKARYEREMKTYIPPKGETKKKFKDPNAP KRLPSAFFLFCSEYRPKIKGEHPGLSIGDVAKKLGEMWNNTAADDKQPYEK KAAKLKEKYEKDIAAYRAICGKPDAAKKGVVKAEKSKKKKEEEEDEEDEED EEEEEDEEDEEDEEEDDDDE) (as determined, for example, using the BLAST program and parameters described herein) and increases inflammation and/or increases release of a proinflammatory cytokine from a cell. In one embodiment, the HMGB polypeptide has one of the above biological activities. Typically, the HMGB polypeptide has both of the above biological activities.

The term “polypeptide” refers to a polymer of amino acids, and not to a specific length; thus, peptides, oligopeptides and proteins are included within the definition of a polypeptide. Preferably, the HMGB polypeptide is a mammalian HMGB polypeptide, for example, a human HMGB1 polypeptide. Preferably, the HMGB polypeptide contains a B box DNA binding domain and/or an A box DNA binding domain and/or an acidic carboxyl terminus as described herein.

Other examples of HMGB polypeptides are described in GenBank Accession Numbers AAA64970, AAB08987, P07155, AAA20508, S29857, P09429, NP_{—}002119, CAA31110, S02826, U00431, X67668, NP-005333, NM_{—}016957, and J04179, the entire teachings of which are incorporated herein by reference. Additional examples of HMGB polypeptides include, but are not limited to, mammalian HMG1 ((HMGB1) as described, for example, in GenBank Accession Number U51677), HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in Genbank Accession Number P05114), HMG 17 (as described, for example, in GenBank Accession Number X13546), HMG1 (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); BMG-X (as described, for example, in GenBank Accession Number D30765) (Xenopus); HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number O00479) (yeast); HMG ½ like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043), as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044); lymphoid specific factor (LEF-1) (as described, for example, in GenBank Accession Number X58636); T-cell specific transcription factor (TCF-1) (as described, for example, in GenBank Accession Number X59869); MTT1 (as described, for example, in GenBank Accession Number M62810); and SP 100-HMG nuclear autoantigen (as described, for example, in GenBank Accession Number U36501). Other examples of HMGB polypeptides include those encoded by nucleic acid sequences having Genbank Accession Numbers AAH81839 (rat high mobility group box 1), NP-990233 (chicken high mobility group box 1), AAN11319 (dog high mobility group B1), AAC27653 (mole high mobility group protein), P07746 (trout high mobility group-T protein), AAA58771 (trout HMG-1), AAQ97791 (zebra fish high-mobility group box 1), AAH01063 (human high-mobility group box 2), and P10103 (cow high mobility group protein 1).

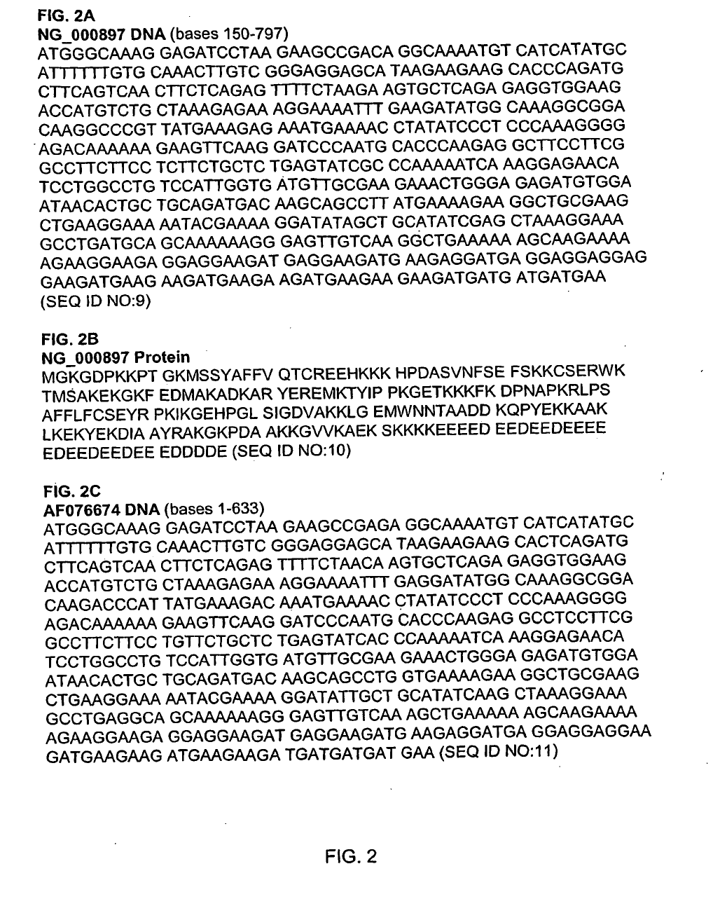

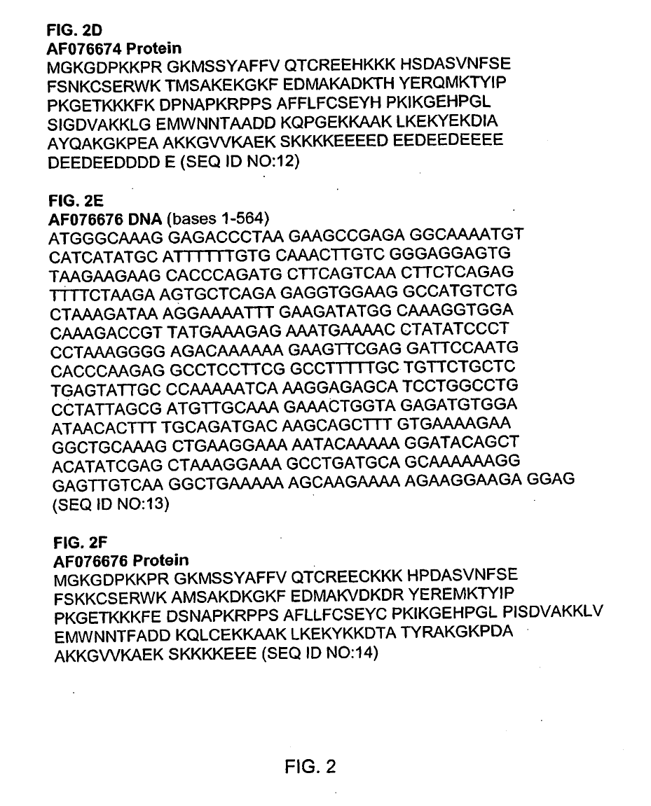

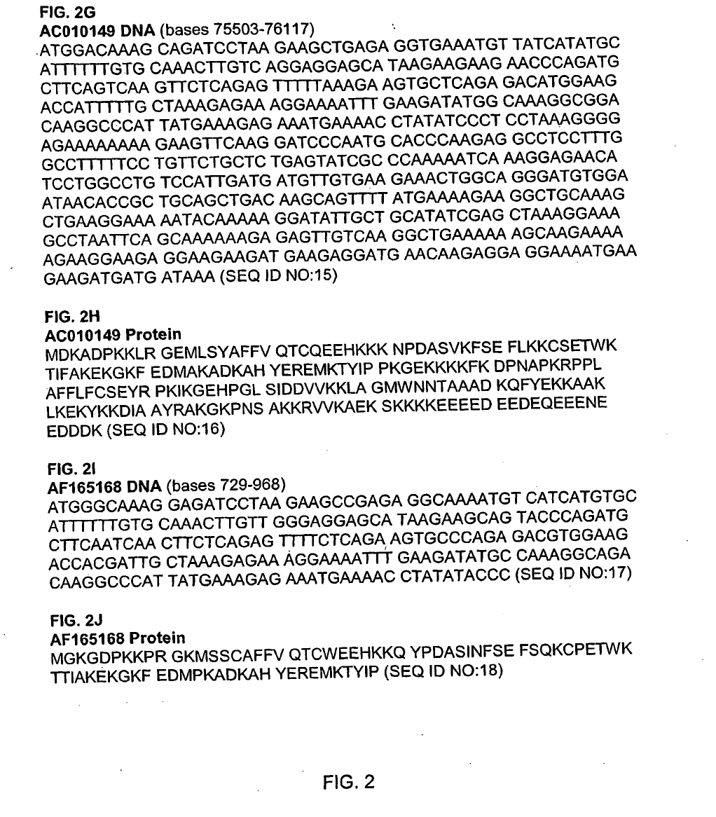

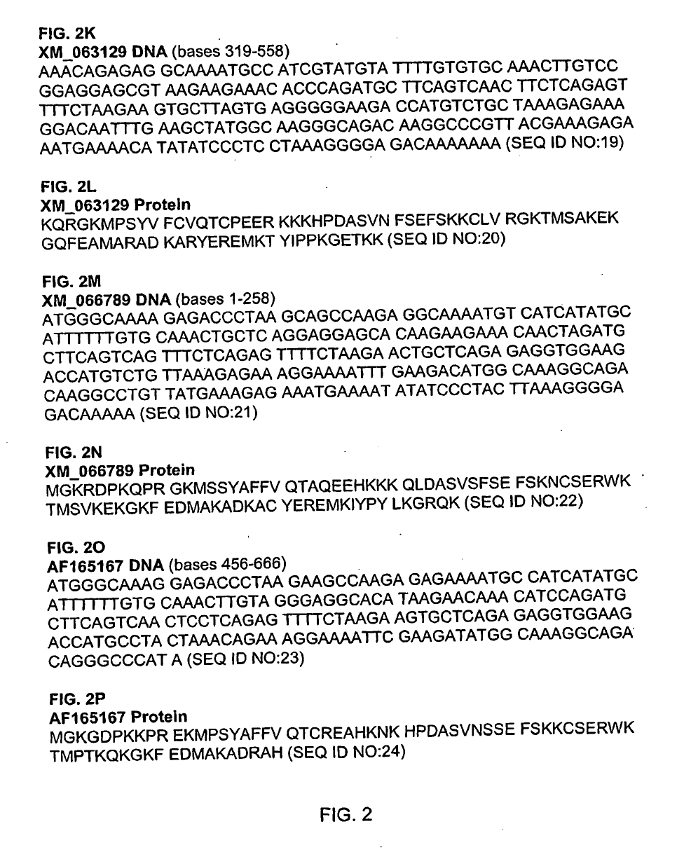

Other examples of HMGB proteins are polypeptides encoded by HMGB nucleic acid sequences having GenBank Accession Numbers NG_{—}000897 (HMG1L5) (and in particular by nucleotides 150-797 of NG_{—}000897, as shown in FIGS. 2A and 2B); AF076674 (HMG1L1) (and in particular by nucleotides 1-633 of AF076674, as shown in FIGS. 2C and 2D; AF076676 (HMG1L4) (and in particular by nucleotides 1-564 of AF076676, as shown in FIGS. 2E and 2F); AC010149 (HMG sequence from BAC clone RP11-395A23) (and in particular by nucleotides 75503-76117 of AC010149, as shown in FIGS. 2G and 2H); AF165168 (HMG1L9) (and in particular by nucleotides 729-968 of AF165168, as shown in FIGS. 2I and 2J); X063129 (LOC122441) (and in particular by nucleotides 319-558 of XM_{—}063129, as shown in FIGS. 2K and 2L); XM_{—}066789 (LOC139603) (and in particular by nucleotides 1-258 of XM_{—}066789, as shown in FIGS. 2M and 2N); and AF165167 (HMG1L8) (and in particular by nucleotides 456-666 of AF165167, as shown in FIGS. 2O and 2P).

Optionally, the HMGB polypeptide is a substantially pure, or substantially pure and isolated, polypeptide that has been separated from components that naturally accompany it. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.” It is understood, however, that preparations in which the polypeptide is not purified to homogeneity are useful. For example, the polypeptide may be in an unpurified form, for example, in a cell, cell milieu, or cell extract. The critical feature is that the preparation allows for the desired function of the polypeptide, even in the presence of considerable amounts of other components.

HMGB polypeptides can be purified from cells that naturally express them, purified from cells that have been altered to express them (recombinant), or synthesized using known protein synthesis methods. In one embodiment, the polypeptide is produced by recombinant DNA techniques. For example, a nucleic acid molecule encoding the polypeptide is cloned into an expression vector, the expression vector is introduced into a host cell and the polypeptide is expressed in the host cell. The polypeptide can then be isolated from the cells by an appropriate purification scheme using standard protein purification techniques.

Inhibitors of functional equivalents of HMGB (proteins or polypeptides that have one or more of the biological activities of an HMGB polypeptide) can also be used in the combination therapy compositions and methods of the present invention. Biologically active fragments, sequence variants, post-translationally modified proteins, and chimeric or fusion proteins comprising HMGB, a biologically active fragment or a variant are examples of functional equivalents of a protein. Variants include a substantially homologous polypeptide encoded by the same genetic locus in an organism, i.e., an allelic variant, as well as other splicing variants. Variants also encompass polypeptides derived from other genetic loci in an organism, but having substantial homology to the protein of interest, for example, an HMGB protein as described herein.

A variant polypeptide can differ in amino acid sequence by one or more substitutions, deletions, insertions, inversions, fusions, and truncations, or a combination of any of these. Further, variant polypeptides can be fully functional or can lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro. Sites that are critical for polypeptide activity can also be determined by structural analysis, such as crystallization, nuclear magnetic resonance or photoaffinity labeling (Smith et al., J. Mol. Biol., 224:899-904 (1992); de Vos et al., Science, 255:306-312 (1992)).

HMGB functional equivalents also include polypeptide fragments of HMGB. Fragments can be derived from an HMGB polypeptide or HMGB variant. As used herein, a fragment comprises at least 6 contiguous amino acids from an HMGB polypeptide. Useful fragments include those that retain-one or more of the biological activities of the polypeptide. Examples of HMGB biologically active fragments include the B box, as well as biologically active fragments of the B box, for example, the first 20 amino acids of the B box (e.g., the first 20 amino acids of SEQ ID NO:5 (SEQ ID NO:44; NAPKRPPSAFFLFCSEYRPK) or SEQ ID NO:8 (SEQ ID NO:45; FKDPNAPKRLPSAFFLFCSE)).

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, or post-translation modification sites. Example of domains include the A box and B box, as described herein.

Fragments can be discrete (not fused to other amino acids or polypeptides) or can be within a larger polypeptide. Further, several fragments can be comprised within a single larger polypeptide. In one embodiment, a fragment designed for expression in a host can have heterologous pre- and pro-polypeptide regions fused to the amino terminus of the polypeptide fragment and an additional region fused to the carboxyl terminus of the fragment.

The compositions and methods of the invention also encompass use of chimeric or fusion polypeptides containing an HMGB polypeptide or a functional equivalent of HMGB (e.g., chimeric or fusion polypeptides containing an HMGB A box or functional equivalent thereof as described herein). These chimeric proteins comprise an HMGB polypeptide or fragment thereof operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the HMGB polypeptide per se. For example, the fusion polypeptide can be a GST-fusion polypeptide in which the polypeptide sequences are fused to the C-terminus of a GST sequence. Other types of fusion polypeptides include, but are not limited to, enzymatic fusion polypeptides, for example, β-galactosidase fusion polypeptides, yeast two-hybrid GAL fusion polypeptides, poly-His fusions, FLAG-tagged fusion polypeptides, GFP fusion polypeptides, and Ig fusion polypeptides. Such fusion polypeptides can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fe is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al., Journal of Molecular Recognition 8:52-58 (1995); Johanson et al., J. Biol. Chem., 270(16):9459-9471 (1995)). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences (e.g., an HMGB polypeptide and another polypeptide) are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST moiety). A nucleic acid molecule encoding an HMGB polypeptide can be cloned into such an expression vector such that the fusion moiety is linked in-frame to the polypeptide.

HMGB functional equivalents can be generated using standard molecular biology techniques and assaying the function using, for example, methods described herein, such as, determining if the functional equivalent, when administered to a cell (e.g., a macrophage), increases release of a proinflammatory cytokine from the cell, as compared to an untreated control cell. In one embodiment, the HMGB functional equivalent has at least 50%, 60%, 70%, 80%, or 90%, of the biological activity of the HMGB1 polypeptide of SEQ ID NO:1.

### HMGB A Boxes

In particular embodiments, the compositions and methods of the present invention encompass HMGB A boxes. As used herein, an “HMGB A box”, also referred to herein as an “A box” (and also known as HMG A box), is a protein or polypeptide that has at least 50%, 60%, 70%, 75%, 80%, 85%, 90%, or 95%, sequence identity to an HMGB A box as described herein, and has one or more of the following biological activities: inhibiting inflammation mediated by HMGB and/or inhibiting release of a proinflammatory cytokine from a cell. In one embodiment, the HMGB A box polypeptide has one of the above biological activities. Typically, the HMGB A box polypeptide has both of the above biological activities. In one embodiment, the A box has at least 50%, 60%, 70%, 75%, 80%, 85%, 90%, or 95%, sequence identity to SEQ ID NO:4 and/or SEQ ID NO:7. In other embodiments, the HMGB A box has no more than 10%, 20%, 25%, 30%, 40%, 50%, 60%, 70%, 80%, or 90%, of the biological activity of full length HMGB. In another embodiment, the HMGB A box amino acid consists of the sequence of SEQ ID NO:4 (FIG. 1D) or SEQ ID NO:7 (PTGKMSSYAFFVQTCREEHKKKHP DASVNFSEFSKKCSERWKTMSAKEKGKFEDMAKADKARYEREMKTYfPPK GET) or the amino acid sequence in the corresponding region of an HMGB protein in a mammal. An HMGB A box is also a recombinantly-produced polypeptide having the same amino acid sequence as the A box sequences described above. The HMGB A box is preferably a vertebrate HMGB A box, for example, a mammalian HMGB A box, more preferably, a mammalian HMGB1 A box, for example, a human HMGB1 A box, and most preferably, the HMGB1 A box comprising, or consisting of, the sequence of SEQ ID NO:4 or SEQ ID NO:7.

An HMGB A box often has no more than about 85 amino acids and no fewer than about 4 amino acids. Examples of polypeptides having A box sequences within them include, but are not limited to, HMGB polypeptides described herein; GenBank Accession Numbers AAA64970, AAB08987, P07155, AAA20508, S29857, P09429, NP_{—}002119, CAA3110, S02826, U00431, X67668, NP_{—}005333, NM_{—}016957, and J04197, mammalian HMG1 ((HMGB1) as described, for example, in GenBank Accession Number U51677), HMG2 ((HMGB2) as described, for example, in GenBank Accession Number M83665), HMG-2A ((HMGB3, HMG4) as described, for example, in GenBank Accession Numbers NM_{—}005342 and NP_{—}005333), HMG14 (as described, for example, in GenBank Accession Number P05114), HMG17 (as described, for example, in GenBank Accession Number X13546), HMG1 (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); HMG-X (as described, for example, in GenBank Accession Number 1D30765) (Xenopus), HMG D (as described, for example, in GenBank Accession Number X71138) and HMG Z (as described, for example, in GenBank Accession Number X71139) (Drosophila); NHP10 protein (HMG protein homolog NHP1) (as described, for example, in GenBank Accession Number Z48008) (yeast); non-histone chromosomal protein (as described, for example, in GenBank Accession Number O00479) (yeast); HMG ½ like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043), as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044); lymphoid specific factor (LEF-1) (as described, for example, in GenBank Accession Number X58636); T-cell specific transcription factor (TCF-1) (as described, for example, in GenBank Accession Number X59869); MTT1 (as described, for example, in GenBank Accession Number M62810) and SP100-HMG nuclear autoantigen (as described, for example, in GenBank Accession Number U36501). Other examples of HMGB polypeptides having A box sequences within them include those encoded by nucleic acid sequences having Genbank Accession Numbers AAH81839 (rat high mobility group box 1), NP_{—}990233 (chicken high mobility group box 1), AAN1319 (dog high mobility group B1), AAC27653 (mole high mobility group protein), P07746 (trout high mobility group-T protein), AAA58771 (trout HMG-1), AAQ97791 (zebra fish high-mobility group box 1), AAH01063 (human high-mobility group box 2), and P10103 (cow high mobility group protein 1).

Other examples of polypeptides having A box sequences within them include, but are not limited to, polypeptides encoded by GenBank Accession Numbers NG_{—}000897 (HMG1L5) (and in particular by nucleotides 150-797 of NG_{—}000897, as shown in FIGS. 2A and 2B); AF076674 (HMG1L1) (and in particular by nucleotides 1-633 of AF076674, as shown in FIGS. 2C and 2D; AF076676 (HMG1L4) (and in particular by nucleotides 1-564 of AF076676, as shown in FIGS. 2E and 2F); AC010149 (HMG sequence from BAC clone RP11-395A23) (and in particular by nucleotides 75503-76117 of AC010149, as shown in FIGS. 2G and 2H); AF165168 (HMG1L9) (and in particular by nucleotides 729-968 of AF165168, as shown in FIGS. 2I and 2J); XM_{—}063129 (LOC122441) (and in particular by nucleotides 319-558 of XM_{—}063129, as shown in FIGS. 2K and 2L); XM_{—}066789 (LOC139603) (and in particular by nucleotides 1-258 of XM_{—}066789, as shown in FIGS. 2M and 2N); and AF165167 (HMG1L8) (and in particular by nucleotides 456-666 of AF165167, as shown in FIGS. 2O and 2P). The A box sequences in such polypeptides can be determined and isolated using methods described herein, for example, by sequence comparisons to A boxes described herein and testing for biological activity using methods described herein and/or other method known in the art.

Additional examples of HMGB A box polypeptide sequences include the following sequences:

(human HMGB1; SEQ ID NO:25)
PDASVNFSEF SKKCSERWKT MSAKEKGKFE DMAKADKARY
EREMKTYIPP KGET;
(human HMGB2; SEQ ID NO:26)
DSSVNFAEF SKKCSERWKT MSAKEKSKFE DMAKSDKARY
DREMKNYVPP KGDK;
(human HMGB3; SEQ ID NO:27)
PEVPVNFAEF SKKCSERWKT VSGKEKSKFD EMAKADKVRY
DREMKDYGPA KGGK;
(HMG1L5; SEQ ID NO:28)
PDASVNFSEF SKKCSERWKT MSAKEKGKFE DMAKADKARY
EREMKTYIPP KGET;
(HMG1L1; SEQ ID NO:29)
SDASVNFSEF SNKCSERWKT MSAKEKGKFE DMAKADKTHY
ERQMKTYIPP KGET;
(HIMG1L4; SEQ ID NO:30)
PDASVNFSEF SKKCSERWKA MSAKDKGKFE DMAKVDKADY
EREMKTYIPP KGET;
(HMG sequence from BAC clone RP11-395A23;
SEQ ID NO:31)
PDASVKFSEF LKKCSETWKT IFAKEKGKFE DMAKADKAHY
EREMKTYIPP KGEK;
(HMG1L9; SEQ ID NO:32)
PDASINFSEF SQKCPETWKT TIAKEKGKFE DMAKADKAHY
EREMKTYIPP KGET;
(HMG1L8; SEQ ID NO:33)
PDASVNSSEF SKKCSERWKT MPTKQGKFED MAKADRAH;
(LOC122441; SEQ ID NO:34)
PDASVNFSEF SKKCLVRGKT MSAKEKGQFE AMARADKARY
EREMKTYIP PKGET;
(LOC139603; SEQ ID NO:35)
LDASVSFSEF SNKCSERWKT MSVKEKGKFE DMAKADKACY
EREMKIYPYL KGRQ;
and
(human HMGB1 A box; SEQ ID NO:36)
GKGDPKKPRG KMSSYAFFVQ TCREEHKKKH PDASVNFSEF
SKKCSERWKT MSAKEKGKFE DMAKADKARY EREMKTYIPP
KGET.

Functional equivalents of HMGB A boxes can also be used in the combination therapy compositions and methods of the present invention. In one embodiment, a functional equivalent of an HMGB A box inhibits release of a proinflammatory-cytokine from a cell treated with an HMGB polypeptide. Examples of HMGB A box functional equivalents include, for example, biologically active fragments, post-translational modifications, variants, or fusion proteins comprising A boxes, as defined herein. A box functional equivalents can be generated using standard molecular biology techniques and assaying the function using known methods, for example, by determining if the fragment, when administered to a cell (e.g., a macrophage) decreases or inhibits release of a proinflammatory cytokine from the cell. In one embodiment, the A box functional equivalent has at least 50%, 60%, 70%, 80%, or 90%, of the biological activity of the HMGB1 polypeptide of SEQ ID NO:4.

Optionally, the HMGB A box polypeptide is a substantially pure, or substantially pure and isolated, polypeptide that has been separated from components that naturally accompany it. The polypeptide may also be in an unpurified form, for example, in a cell, cell milieu, or cell extract. The critical feature is that the preparation allows for the desired function of the polypeptide, even in the presence of considerable amounts of other components.

### HMGB B Boxes

In particular embodiments, the compositions and methods of the present invention comprise antibodies to the HMGB B box or antigen-binding fragments thereof. As used herein, an “HMGB B box”, also referred to herein as a “B box” (and also known as an HMG B box), is a polypeptide that has at least 60%, 65%, 70%, 75%, 80%, 85%, 90%, or 95%, sequence identity to SEQ ID NO:5 and/or SEQ ID NO: 8 (as determined using the BLAST program and parameters described herein), lacks an A box, and has one or more of the following biological activities: increasing inflammation and/or increasing release of a proinflammatory cytokine from a cell. In one embodiment, the HMGB B box polypeptide has one of the above biological activities. Typically, the HMGB B box polypeptide has both of the above biological activities. Preferably, the HMGB B box has at least 25%, 30%, 40%, 50%, 60%, 70%, 80%, or 90%, of the biological activity of full length HMGB. In another embodiment, the HMGB box comprises or consists of the sequence of SEQ ID NO:5 (FIG. 1E) or SEQ ID NO:8 (FKDPNAPKRL PSAFFLFCSE YRPKIKG EHPGLSIGDVAKYLGEMWNNTAADDKQPYEKKAAKLKEKYEKDIAAY) or the amino acid sequence in the corresponding region of an HMGB protein in a mammal.

Preferably, the HMGB B box is a mammalian HMGB B box, for example, a human HMGB1 B box. An HMGB B box often has no more than about 85 amino acids and no fewer than about 4 amino acids. Examples of polypeptides having B box sequences within them include, but are not limited to, HMGB polypeptides described herein; GenBank Accession Numbers AAA64970, AAB08987, P07155, AAA20508, S29857, P09429, NP_{—}002119, CAA31110, S02826, U00431, X67668, NP_{—}005333, NM_{—}016957, and J04197, mammalian HMG1 ((HMGB1) as described, for example, in GenBank Accession Number U51677), HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number P05114), HMG17 (as described, for example, in GenBank Accession Number X13546), HMG1 (as described, for example, in GenBank Accession Number L17131), and HMGY (as described, for example, in GenBank Accession Number M23618); noumammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus), HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number 000479) (yeast); HMG ½ like protein (as described, for example, in GenBank Accession Number Z1540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043), as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044); lymphoid specific factor (LEF-1) (as described, for example, in GenBank Accession Number X58636); T-ell specific transcription factor (TCF-1) (as described, for example, in GenBank Accession Number X59869); MTT1 (as described, for example, in GenBank Accession Number M62810); and SP100-HMGB nuclear autoantigen (as described, for example, in GenBank Accession Number U36501). Other examples of polypeptides having B box sequences within them include, HMGB polypeptides encoded by nucleic acid sequences having Genbank Accession Numbers AAH81839 (rat high mobility group box 1), NP-990233 (chicken high mobility group box 1), AAN11319 (dog high mobility group B1), AAC27653 (mole high mobility group protein), P07746 (trout high mobility group-T protein), AAA58771 (trout HMG-1), AAQ97791 (zebra fish high-mobility group box 1), AAH01063 (human high-mobility group box 2), and P10103 (cow high mobility group protein 1).

Other examples of polypeptides having B box sequences within them include, but are not limited to, polypeptides encoded by GenBank Accession Numbers NG_{—}000897 (HMG1L5) (and in particular by nucleotides 150-797 of NG_{—}000897, as shown in FIGS. 2A and 2B); AF076674 (HMG1L1) (and in particular by nucleotides 1-633 of AF076674, as shown in FIGS. 2C and 2D; AF076676 (HMG1L4) (and in particular by nucleotides 1-564 of AF076676, as shown in FIGS. 2E and 2F); AC010149 (HMG sequence from BAC clone RP11-395A23) (and in particular by nucleotides 75503-76117 of AC001149, as shown in FIGS. 2G and 2H) The B box sequences in such polypeptides can be determined and isolated using methods described herein, for example, by sequence comparisons to B boxes described herein and testing for biological activity.

Examples of HMGB B box polypeptide sequences include the following sequences:

(human HMGB1; SEQ ID NO:37)
FKDPNAPKRP PSAFFLFCSE YRPKIKGEHP GLSIGDVAKK
LGEMWNNTAA DDKQPYEKKA AKLKEKYEKD IAAY;
(human HMGB2; SEQ ID NO:38)
KKDPNAPKRP PSAFFLFCSE HRPKIKSEHP GLSIGDTAKK
LGEMWSEQSA KDKQPYEQKA AKLKEKYEKD IAAY;
(HMG1L5; SEQ ID NO:39)
FKDPNAPKRL PSAFFLFCSE YRPKIKGEHP GLSIGDVAKK
LGEMWNNTAA DDKQPYEKKA AKLKEKYEKD IAAY;
(HMG1L1; SEQ ID NO:40)
FKDPNAPKRP PSAFFLFCSE YHPKIKGEHP GLSIGDVAKK
LGEMWNNTAA DDKQPGEKKA AKLKEKYEKD IAAY;
(HMG1L4; SEQ ID NO:41)
FKDSNAPKRP PSAFLLFCSE YCPKIKGEHP GLPISDVAKK
LVEMWNNTFA DDKQLCEKKA AKLKEKYKKD TATY;
(HMG sequence from BAC clone RP11-359A23;
SEQ ID NO:42)
FKDPNAPKRP PSAFFLFCSE YRPKIKGEHP GLSIGDVVKK
LAGMWNNTAA ADKQFYEKKA AKLKEKYKKD IAAY;
and
(human HMGB1 box; SEQ ID NO:43)
FKDPNAPKRP PSAFFLFCSE YRPKIKGEHP GLSIGDVAKK
LGEMWNNTAA DDKQPYEKKA AKLKEKYEKD IAAYRAKGKP
DAAKKGVVKA EK.

Antibodies to functional equivalents of HMGB B boxes can also be used in the combination therapy compositions and methods of the present invention. Examples of HMGB B box functional equivalents include, for example, biologically active fragments, post-translational modifications, variants, or fusion proteins comprising B boxes, as defined herein. B box functional equivalents can be generated using standard molecular biology techniques and assaying the function using known methods, for example, by determining if the functional equivalent, when administered to a cell (e.g., a macrophage), increases release of a proinflammatory cytokine from the cell. In one embodiment, the B box functional equivalent has at least 50%, 60%, 70%, 80%, or 90%, of the biological activity of the B box polypeptide of SEQ ID NO:5. Preferred examples of B box biological equivalents are polypeptides comprising, or consisting of, the first 20 amino acids of the B box (e.g., the first 20 amino acids of SEQ ID NO:5 (SEQ ID NO:44) or SEQ ID NO:8 (SEQ ID NO:45).

Optionally, the HMGB B box polypeptide is a substantially pure, or substantially pure and isolated, polypeptide that has been separated from components that naturally accompany it. Alternatively, the polypeptide may be in an unpurified form, for example, in a cell, cell milieu, or cell extract. The critical feature is that the preparation allows for the desired function of the polypeptide, even in the presence of considerable amounts of other components.

HMGB, HMGB A box, and/or HMGB B box functional equivalents, either naturally occurring or non-naturally occurring, include polypeptides that have sequence identity to the HMGB polypeptides, HMGB A boxes, and HMGB B boxes described herein. As used herein, two polypeptides (or regions of the polypeptides) are substantially homologous or identical when the amino acid sequences are at least about 60%, 70%, 75%, 80%, 85%, 90%, or 95% or more, homologous or identical. The percent identity of two amino acid sequences (or two nucleic acid sequences) can be determined by aligning the sequences for optimal comparison purposes (e.g., gaps can be introduced into one or both of the sequences). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the HMGB polypeptide, HMGB A box polypeptide, or HMGB B box polypeptide aligned for comparison purposes is at least 30%, preferably at least 40%, more preferably at least 60%, and even more preferably at least 70%, 80%, 90%, or 100%, of the length of the reference sequence, for example, those sequences provided in FIGS. 1A-1E, FIGS. 2A-2P, and SEQ ID NOS:25-43. The actual comparison of the two sequences can be accomplished by well-known methods, for example, using a mathematical algorithm. A preferred, non-limiting example of such a mathematical algorithm is described in Karlin et al. Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10:3-5 (1994)); and FASTA described in Pearson and Lipman (Proc. Natl. Acad. Sci. USA, 85:2444-2448 (1988)).

In another embodiment, the percent identity between two amino acid sequences can be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

As used herein, a “cytokine” is a soluble protein or peptide that is naturally produced by mammalian cells and that regulates immune responses and mediates cell-cell interactions. Cytokines can, either under normal or pathological conditions, modulate the functional activities of individual cells and tissues. A proinflammatory cytokine is a cytokine that is capable of causing one or more of the following physiological reactions associated with inflammation or inflammatory conditions: vasodilation, hyperemia, increased permeability of vessels with associated edema, accumulation of granulocytes and mononuclear phagocytes, and deposition of fibrin. In some cases, the proinflammatory cytokine can also cause apoptosis, such as in chronic heart failure, where TNF has been shown to stimulate cardiomyocyte apoptosis (Pukki, Ann. Med. 29:339-343 (1997); and Tsutsui et al., Immunol. Rev. 174:192-209 (2000)).

Nonlimiting examples of proinflammatory cytokines are tumor necrosis factor (TNF), interleukin (IL)-1α, IL-1β, IL-6, IL-8, IL-18, interferon γ, HMG-1, and macrophage migration inhibitory factor (MIF). Proinflammatory cytokines are to be distinguished from anti-inflammatory cytokines, such as IL-4, IL-10, and IL-13, which are not mediators of inflammation.

In many instances, proinflammatory cytokines are produced in an inflammatory cytokine cascade, defined herein as an in vivo release of at least one proinflammatory cytokine in a mammal, wherein the cytokine release, directly or indirectly (e.g., through activation of, production of, or release of, one or more cytokines or other molecules involved in inflammation from a cell), stimulates a physiological condition of the mammal. Thus, an inflammatory cytokine cascade is inhibited in embodiments of the invention where proinflammatory cytokine release causes a deleterious physiological condition.

Inhibition of release of a proinflammatory cytokine from a cell can be measured according to methods known to one skilled in the art. For example, TNF release from a cell can be measured using a standard murine fibroblast L929 (ATCC, American Type Culture Collection, Manassas, Va.) cytotoxicity bioassay (Bianchi et al., J. Exp. Med. 183:927-936 (1996) with the minimum detectable concentration of 30 μg/ml. The L929 cytotoxicity bioassay is carried out as follows. RAW 264.7 cells are cultured in RPMI 1640 medium (Life Technologies, Grand Island, N.Y.) supplemented with 10% fetal bovine serum (Gemini, Catabasas, Calif.), penicillin and streptomycin (Life Technologies, Grand Island, N.Y.). Polymyxin (Sigma, St. Louis, Mo.) is added at 100 units/ml to suppress the activity of any contaminating LPS. Cells are incubated with the combination therapy compositions described herein in Opti-MEM I medium for 8 hours, and conditioned supernatants (containing TNF which has been released from the cells) are collected. TNF that has been released from the cells is measured using a standard murine fibroblast L929 (ATCC) cytotoxicity bioassay (Bianchi et al., supra) with the minimum detectable concentration of 30 pg/ml. Recombinant mouse TNF is obtained from R&D Systems Inc. (Minneapolis, Minn.) and is used as a control in these experiments. Methods for measuring release of other cytokines from cells are known in the art.

Inflammatory cytokine cascades contribute to deleterious characteristics, including inflammatory conditions and cellular apoptosis. The compositions and methods disclosed herein can be used to inhibit an inflammatory condition. In one embodiment, the inflammatory condition to be treated is one in which the inflammatory cytokine cascade causes a systemic reaction, such as endotoxic shock. In another embodiment, the inflammatory condition to be treated is one in which the inflammatory cytokine cascade is mediated by a localized inflammatory cytokine cascade, such as rheumatoid arthritis. In another embodiment, the inflammatory condition is selected from the group consisting of ileus, appendicitis, peptic, gastric or duodenal ulcers, inflammatory bowel disease, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion ischemia, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, uretiritis, bronchitis, emphysema, rhinitis, cystic fibrosis, pneumonitis, pneumoultramicroscopicsilicovolcanoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, restenosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, arthritis, rheumatoid arthritis, synovitis, myasthenia gravis, thryoiditis, systemic lupus erythematosus, Goodpasture's syndrome, Behcets's syndrome, chronic obstructive pulmonary disease, psoriasis, allograft rejection, graft-versus-host disease, Type I diabetes, ankylosing spondylitis, Berger's disease, Retier's syndrome, and Hodgkins disease.

In other embodiments, the condition is selected from one or more of the group consisting of sepsis, peritonitis, pancreatitis, inflammatory bowel disease, ileus, ulcerative colitis, Crohn's disease, ischemia, for example, myocardial ischemia, organic ischemia, or reperfusion ischemia, cachexia, burns, adult respiratory distress syndrome, multiple sclerosis, atherosclerosis, restenosis, arthritis, rheumatoid arthritis, asthma, systemic lupus erythematosus, adult respiratory distress syndrome, chronic obstructive pulmonary disease, psoriasis, Behcet's syndrome, psoriasis, allograft rejection and graft-versus-host disease. Where the condition is allograft rejection, the composition may advantageously also include an immunosuppressant that is used to inhibit allograft rejection, such as cyclosporin.

When referring to the effect of any of the compositions or methods of the invention on the release of proinflammatory cytokines, the use of the terms “inhibit” or “decrease” encompasses at least a small but measurable reduction in proinflammatory cytokine release. In preferred embodiments, the release of the proinflammatory cytokine is inhibited by at least 10%, 20%, 25%, 30%, 40%, 50%, 75%, 80%, or 90%, over non-treated controls. Inhibition can be assessed using methods described herein or other methods known in the art. Such reductions in proinflammatory cytokine release are capable of reducing the deleterious effects of an inflammatory cytokine cascade in in vivo embodiments.

### Antibodies to HMGB, HMGB B Box and HMGB A Box Polypeptides

The present invention is directed in part to compositions including, and methods utilizing, antibodies and antigen-binding fragments thereof that bind to an HMGB polypeptide or a biologically active fragment thereof (anti-HMGB antibodies). These antibodies and antigen-binding fragments can be combined with an agent that inhibits caspase activity. The anti-HMGB antibodies and antigen-binding fragments can be neutralizing antibodies or antigen-binding fragments (i.e., they can inhibit a biological activity of an HMG polypeptide or a fragment thereof, for example, the release of a proinflammatory cytokine from a vertebrate cell induced by HMGB). The invention also encompasses antibodies and antigen-binding fragments that selectively bind to an HMGB B box or a fragment thereof, but do not selectively bind to non-B box epitopes of HMGB (anti-HMGB B box antibodies and antigen-binding fragments thereof). The invention further encompasses antibodies and antigen-binding fragments that selectively bind to an HMGB A box or a functional equivalent thereof, but do not selectively bind to non-A box epitopes of HMGB (anti-HMGB A box antibodies and antigen-binding fragments thereof). In these embodiments, the antibodies and antigen-binding fragments can also be neutralizing antibodies and antigen-binding fragments (i.e., they can inhibit a biological activity of a HMGB polypeptide or a B box polypeptide or fragment thereof, for example, the release of a proinflammatory cytokine from a vertebrate cell induced by HMGB). Antibodies to HMGB have been shown to inhibit release of a proinflammatory cytokine from a cell treated with an HMGB polypeptide (see, for example, PCT publication WO 02/092004). Such antibodies can be combined with one or more agents that inhibit caspase activity.

The term “antibody” or “purified antibody” as used herein refers to immunoglobulin molecules. The term “antigen-binding fragment” or “purified antigen-binding fragment” as used herein refers to immunologically active portions of immunoglobulin molecules, i.e., molecules that contain an antigen binding site that selectively bind to an antigen. A molecule that selectively binds to a polypeptide is a molecule that binds to that polypeptide or a fragment thereof, but does not substantially bind other molecules in a sample, e.g., a biological sample that naturally contains the polypeptide. Preferably the antibody is at least 60%, by weight, free from proteins and naturally occurring organic molecules with which it naturally associates. More preferably, the antibody preparation is at least 75% or 90%, and most preferably, 99%, by weight, antibody. Examples of immunologically active portions of immunoglobulin molecules include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

In particular embodiments, the compositions of the invention include polyclonal and/or monoclonal antibodies that selectively bind to an HMGB B box polypeptide or an HMGB A box polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described herein by immunizing a suitable subject with a desired immunogen, e.g., an HMGB polypeptide, an HMGB B box polypeptide, an HMGB A box polypeptide or fragments thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, Coligan et al., (eds.) John Wiley & Sons, Inc., New York, N.Y., 1994). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide described herein.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature, 266:55052, 1977; R. H. Kenneth, in Monoclonal Antibodies: A New Dimension In Biological Analyses, Plenum Publishing Corp., New York, N.Y., 1980; and Lerner, Yale J. Biol. Med. 54:387-402, 1981). Moreover, the ordinarily skilled worker will appreciate that there are many variations of such methods that also would be useful.

In one alternative to preparing monoclonal antibody-secreting hybridomas, a monoclonal antibody to an HMGB polypeptide, an HMGB B box polypeptide or an HMGB A box polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372, 1991; Hay et al., Hum. Antibodies Hybridomas 3:81-85, 1992; Huse et al., Science 246:1275-1281, 1989; and Griffiths et al., EMBO J. 12:725-734, 1993.

Single chain antibodies, and recombinant antibodies, such as chimeric, humanized, primatized (CDR-grafted) or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies. Techniques for the production of single chain antibodies (U.S. Pat. No. 4,946,778) can be adapted to produce single chain antibodies to the HMGB polypeptides or HMGB B box polypeptides or fragments thereof. Also, transgenic mice, or other organisms such as other mammals, may be used to express humanized antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213).

If the antibody is used therapeutically in in vivo applications, the antibody can be modified to make it less immunogenic. For example, if the individual is human the antibody is preferably “humanized”; where the complementarity determining region(s) (CDRs) of the antibody is transplanted into a human antibody (for example, as described in Jones et al., Nature 321:522-525, 1986; and Tempest et al., Biotechnology 9:266-273, 1991). The antibody can be a humanized antibody comprising one or more immunoglobulin chains, said antibody comprising a CDR of nonhuman origin (e.g., one or more CDRs derived from an antibody of nonhuman origin) and a framework region derived from a light and/or heavy chain of human origin (e.g., CDR-grafted antibodies with or without framework changes). In one embodiment, the antibody or antigen-binding fragment thereof comprises the light chain CDRs (CDR1, CDR2 and CDR3) and heavy chain CDRs (CDR1, CDR2 and CDR3) of a particular immunoglobulin. In another embodiment, the antibody or antigen-binding fragment further comprises a human framework region.

Because vertebrate HMGB polypeptides, HMGB B boxes and HMGB A boxes show a high degree of sequence conservation, it is believed that antibodies that bind to vertebrate HMGB polypeptides, HMGB B boxes or HMGB A boxes, in general, can induce release of a proinflammatory cytokine from a vertebrate cell. Therefore, antibodies against vertebrate HMGB polypeptides or HMGB B boxes without limitation are within the scope of the invention. In one embodiment, the antibodies are neutralizing antibodies.

Phage display technology can also be utilized to select antibody genes with binding activities towards the polypeptide either from repertoires of PCR amplified v-genes of lymphocytes from humans screened for possessing anti-B box antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352: 624-628, 1991).

Other suitable methods of producing or isolating antibodies of the requisite specificity (e.g., human antibodies or antigen-binding fragments) can be used, including, for example, methods that select recombinant antibody from a library (e.g., a phage display library). Transgenic animals capable of producing a repertoire of human antibodies (e.g., Xenomouse® (Abgenix, Fremont, Calif.)) can be produced using suitable methods (see e.g., Jakobovits, et al., Proc. Natl. Acad. Sci. USA, 90: 2551-2555 (1993); Jakobovits, et al., Nature, 362: 255-258 (1993)). Additional methods that are suitable for production of transgenic animals capable of producing a repertoire of human antibodies have been described (e.g., Lonberg, et al., U.S. Pat. No. 5,545,806; Surani, et al., U.S. Pat. No. 5,545,807; Lonberg, et al., WO 97/13852).

When the antibodies are obtained that specifically bind to HMGB epitopes, HMGB B box epitopes and/or HMGB A box epitopes, they can then be screened without undue experimentation for the ability to inhibit release of a proinflammatory cytokine using standard methods. Anti-HMGB antibodies, anti-HMGB B box antibodies and anti-HMGB A box antibodies that can inhibit the production of any single proinflammatory cytokine, and/or inhibit the release of a proinflammatory cytokine from a cell, and/or inhibit the a condition characterized by activation of an inflammatory cytokine cascade are within the scope of the present invention. Preferably, the antibodies can inhibit the production of TNF, IL-1β, or IL-6.

Polyclonal antibodies raised against HMGB have been produced (see, for example, U.S. Pat. No. 6,468,555 B1, the entire teachings of which are incorporated herein by reference). These antibodies have been shown to inhibit release of a proinflammatory cytokine from a cell, and to treat inflammation.

Polyclonal antibodies against the HMGB1 B box have been raised in rabbits (Cocalico Biologicals, Inc., Reamstown, Pa.) and assayed for titer by immunoblotting. IgG was purified from anti-HMGB1 antiserum using Protein A agarose according to manufacturer's instructions (Pierce, Rockford, Ill.) (see, for example, PCT Publication No. WO 02/092004). Anti-HMGB1 B box antibodies were affinity purified using cyanogen bromide activated Sepharose beads (Cocalico Biological, Inc.). Non-immune rabbit IgG was purchased from Sigma (St. Louis, Mo.). Antibodies detected full length HMGB1 and HMGB1 B box in immunoassays, but did not cross react with TNF, IL-1 or IL-6. These HMGB1 B box antibodies also inhibited release of a proinflammatory cytokine from a cell and provided protection against sepsis induced by cecal ligation and puncture.

Monoclonal antibodies to HMGB1 are known in the art, and are taught, for example, in U.S. Provisional Application No. 60/502,568, entitled “Monoclonal Antibodies Against HMGB1”, by Walter Newman, Shixin Qin, Theresa O'Keefe and Robert Obar, filed on Sep. 11, 2003, Attorney Docket No. 3258.1033-000; the entire teachings of which are incorporated herein by reference. Particular monoclonal antibodies to HMGB1 include, e.g., 6E6 HMGB1 mAb, 2E11 HMGB1 mAb, 6H9 HMGB1 mAb, 10D4 HMGB1 mAb and 2G7 HMGB1 mAb.

6E6 HMGB1 mAb, also referred to as 6E6-7-1-1 or 6E6, can be produced by murine hybridoma 6E6 HMGB1 mAb, which was deposited on Sep. 3, 2003, on behalf of Critical Therapeutics, Inc., 675 Massachusetts Avenue, 14t Floor, Cambridge, Mass. 02139, U.S.A., at the American Type Culture Collection, 10801 University Boulevard, Manassas, Va. 20110, U.S.A., under Accession No. PTA-5433.

2E11 HMGB1 mAb, also referred to as 2E11-1-1-2 or 2E11, can be produced by murine hybridoma 2E11 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5431.

6H9 HMGB1 mAb, also referred to as 6H9-1-1-2 or 6H9, can be produced by murine hybridoma 6H9 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5434.

10D4 HMGB1 mAb, also referred to as 10D4-1-1-1-2 or 10D4, can be produced by murine hybridoma 110D4 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5435.

2G7 HMGB1 mAb, also referred to as 3-2G7-1-1-1 or 2G7, can be produced by murine hybridoma 2G7 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5432.

As described herein, in certain embodiments the compositions and methods of the invention comprise or utilize antibodies or antigen-binding fragments thereof, that bind an HMGB polypeptide or fragment thereof (e.g., an HMGB B box or a biologically active fragment thereof, an HMGB A box or a biologically active fragment thereof). Such HMGB polypeptides include, e.g., those HMGB polypeptides described herein. In one embodiment, the antibody or antigen-binding fragment binds a mammalian HMGB polypeptide. In another embodiment, the antibody or antigen-binding fragment binds an HMGB1 polypeptide. In yet another embodiment, the antibody or antigen-binding fragment binds an HMGB1 polypeptide consisting of SEQ ID NO:1.

In one embodiment, the antibody or antigen-binding fragment binds an HMGB B box or a biologically active fragment thereof. In another embodiment, the antibody or antigen-binding fragment binds an HMGB B box consisting of SEQ ID NO:5. In yet another embodiment, the antibody or antigen-binding fragment binds a biologically active fragment of an HMGB B Box consisting of SEQ ID NO:45.

In one embodiment, the antibody or antigen-binding fragment binds an HMGB A box or a biologically active fragment thereof. In another embodiment, the antibody or antigen-binding fragment binds an HMGB A box consisting of SEQ ID NO:4. In yet another embodiment, the antibody or antigen-binding fragment binds a biologically active fragment of an HMGB A Box.

Other Inhibitors of HMGB Receptor Binding, HMGB signaling, HMGB-Mediated Inflammation and/or HMGB-Mediated Release of a Proinflammatory Cytokine

As described herein, agents that inhibit HMGB biological activity include, e.g., agents that inhibit HMGB receptor binding, agents that inhibit HMGB signaling, agents that inhibit HMGB-mediated inflammation, agents that inhibit HMGB-mediated release of a proinflammatory cytokine, agents that inhibit HMGB-mediated activation of antigen presenting cells; and agents that inhibit HMGB-mediated induction of smooth muscle cell migration. Such agents include, e.g., agents that interact directly with HMGB and inhibit HMGB biological activity and agents that do not interact directly with HMGB and inhibit HMGB biological activity.

In one embodiment, the agent that inhibits HMGB biological activity inhibits one or more activities selected from the group consisting of: inhibiting HMGB receptor binding; inhibiting HMGB signaling; inhibiting HMGB-mediated inflammation; inhibiting HMGB-mediated release of a proinflammatory cytokine; inhibiting HMGB-mediated activation of antigen presenting cells; and inhibiting HMGB-mediated induction of smooth muscle cell migration. In one embodiment, the invention is directed to combination therapy compositions comprising an inhibitor of HMGB biological activity and a caspase inhibitor. Such combination therapy compositions can be used for the treatment of inflammatory conditions, as described herein.

Inhibitors of HMGB biological activity include, e.g., polypeptides comprising a high mobility group box (HMGB) A box (as described herein), antibodies to HMGB, HMGB B boxes and/or HMGB A boxes and antigen-binding fragments thereof (as described herein), HMGB small molecule inhibitors (e.g., ethyl pyruvate, certain derivatives of isoxazole, isoxazolidine, isothiazole and isothiazolidine compounds), cholinergic agonists (e.g., α7 nicotinic receptor agonists), antibodies to TLR2, soluble TLR2, TLR2 small molecule antagonists, TLR2 dominant mutant proteins, antibodies to TLR4, soluble TLR4, TLR4 small molecule antagonists, TLR4 dominant mutant proteins, antibodies to RAGE, soluble RAGE, RAGE small molecule antagonists (e.g., as taught in PCT Publication Nos. WO 01/99210, WO 02/069965 and WO 03/075921 and U.S. Published Application No. US 2002/0193432A1), and RAGE dominant mutant proteins. Inhibitors of HMGB receptor binding and/or signaling also include, e.g., antisense and small double-stranded interfering RNA (RNA interference (RNAi) that target HMGB, TLR2, TLR4 and/or RAGE proteins.

### HMGB Small Molecule Inhibitors

In one embodiment, the inhibitor of HMGB biological activity is an HMGB small molecule inhibitor. As used herein, an HMGB small molecule inhibitor is a molecule that antagonizes production of HMGB and/or one or more biological activities of HMGB (e.g., HMGB receptor binding, HMGB-mediated signaling, HMGB-mediated increase in inflammation, HMGB-mediated increase in release of a proinflammatory cytokine from a cell, HMGB-mediated activation of antigen presenting cells, HMGB-mediated induction of smooth muscle cell migration). Such HMGB small molecule inhibitors include those small molecule antagonists that bind directly to HMGB, thereby inhibiting HMGB biological activity, as well as those small molecule inhibitors that do not bind to HMGB but antagonize production of HMGB and/or one or more biological activities of HMGB. HMGB small molecule inhibitors typically have a molecular weight of 1000 or less, 500 or less, 250 or less or 100 or less. Suitable HMGB small molecule inhibitors include but are not limited to, an ester of an alpha-ketoalkanoic acid including, for example, ethyl pyruvate (see, e.g., PCT Publication WO 02/074301; the entire teachings of which are incorporated herein by reference) and certain derivatives of isoxazole, isoxazolidine, isothiazole and isothiazolidine compounds (e.g., as taught in WO 2005034952).

For example, it has been shown that an ester of an alpha-ketoalkanoic acid can inhibit the release of proinflammatory cytokines such as TNF, IL-1β and HMGB1. See, e.g., PCT Publication WO 02/074301, the entire teachings of which are incorporated herein by reference. Therefore, in one embodiment of the invention, the HMGB small molecule inhibitor is an ester of an alpha-ketoalkanoic acid. In another embodiment, the HMGB small molecule inhibitor is an ester of a C3 to C8, straight chained or branched alpha-ketoalkanoic acid. In an additional embodiment, the HMGB small molecule inhibitor is selected from the group consisting of alpha-keto-butyrate, alpha-ketopentanoate, alpha-keto-3-methyl-butyrate, alpha-keto-4-methyl-pentanoate or alpha-keto-hexanoate. A variety of groups are suitable for the ester portion of the molecule, e.g., alkyl, aralkyl, alkoxyl, carboxyalkyl, glyceryl or dihydroxyacetone. Specific examples include ethyl, propyl, butyl, carboxymethyl, acetoxymethyl, carbethoxymethyl and ethoxymethyl. Ethyl esters are preferred. In a further embodiment, the HMGB small molecule inhibitor is an ethyl, propyl, butyl, carboxymethyl, acetoxymethyl, carbethoxymethyl and ethoxymethyl ester. In an additional preferred embodiment, the HMGB small molecule inhibitor is an ester of pyruvic acid. In a further preferred embodiment, the HMGB small molecule inhibitor is ethyl pyruvate. Thiolesters (e.g., wherein the thiol portion is cysteine or homocysteine) are also included.

In another preferred embodiment, the HMGB small molecule inhibitor is selected from the group consisting of ethyl pyruvate, propyl pyruvate, carboxymethyl pyruvate, acetoxymethyl pyruvate, carbethoxymethyl pyruvate, ethoxymethyl pyruvate, ethyl alpha-keto-butyrate, ethyl alpha-keto-pentanoate, ethyl alpha-keto-4-methyl-pentanoate and ethyl-keto-hexanoate. In an additional preferred embodiment, the HMGB small molecule inhibitor is ethyl pyruvate.

### Cholinergic Agonists

It has been discovered that cholinergic agonists can inhibit the release of HMGB1 from macrophages (see U.S. Pat. No. 6,610,713; the teachings of which are incorporated herein by reference in their entirety). Thus, in particular embodiments, the combination therapy compositions of the invention comprise an inhibitor of HMGB biological activity that is a cholinergic agonist. As used herein, a cholinergic agonist is a compound that binds to cells expressing cholinergic receptor activity. Nonlimiting examples of cholinergic agonists include acetylcholine, nicotine, muscarine, carbachol, galantamine, arecoline, cevimeline and levamisole. In one embodiment, the cholinergic agonist is nicotine.

It has further been discovered that cholinergic agonists (e.g., nicotinic cholinergic agonists) that are selective for the α7 nicotinic receptor can inhibit the release of a proinflammatory cytokine (e.g., TNF) from macrophages (see U.S. Published Application No. 2004/0204355; the teachings of which are incorporated herein by reference in their entirety). As used herein, an “α7 receptor”, “α7 nicotinic receptor” or “07 cholinergic receptor” is a receptor comprising α7 subunit. The receptor can comprise only α7 subunits; alternatively the receptor comprises 07 subunit(s) and subunit(s) from other cholinergic receptor subtypes. In one embodiment, the receptor is a homopentamer. In another embodiment, the receptor is a heteropentamer. As used herein, an “α7 agonist”, “α7 receptor agonist”, “α7 nicotinic receptor agonist” or “α7 cholinergic receptor agonist” is a compound that binds to a receptor comprising an α7 subunit, in vivo or in vitro, and induces the receptor to perform its physiological function. In one embodiment, an α7 receptor agonist inhibits release of proinflammatory cytokines from cells expressing nicotinic receptors comprising α7 subunits when the cell is otherwise stimulated to release those proinflammatory cytokines. The skilled artisan can determine whether any particular compound is an α7 receptor agonist by any of several well known methods, for example, as taught in U.S. Published Application No. 2004/0204355.

Thus, in one embodiment, the combination therapy compositions of the invention comprise an inhibitor of HMGB biological activity that is an α7 nicotinic receptor agonist. Any α7 agonist, now known or later discovered, would be expected to inhibit the release of proinflammatory cytokines (e.g., HMGB1) from mammalian cells. In preferred embodiments, the cholinergic agonist is not otherwise toxic to the cell at useful concentrations. In more preferred embodiments, the cholinergic agonist has been used therapeutically in vivo or is naturally produced by mammalian cells. Nonlimiting examples of α7 agonists include acetylcholine, muscarine, nicotine, 3-2,4-dimethoxybenzylidine anabaseine (DMXB-A, also known as GTS-21) (Kem, W. R., et al., J. Pharmacol. Exp. Ther. 283:979-992 (1997); Simosky, J. K., et al., Biol. Psychiatry 50:493-500 (2001)), trans-3-cinnamylidene anabaseine, trans-3-(2-methoxy-cinnamylidene)anabaseine, trans-3-(4-methoxycinnamylidene)anabaseine, choline, cocaine methiodide (Francis, M. M., et al., Mol. Pharmacol. 60:71-79 (2001)).

In one embodiment, the cholinergic agonist is an agonist that is selective or specific for α7, since such an agonist would be expected to cause fewer side effects than a non-specific cholinergic agonist (e.g., nicotine), to a subject that is being treated for inflammation. As used herein, an agonist is selective for α7 if that agonist is an agonist that activates α7 to a greater extent than the agonist activates at least one other nicotinic receptor. Such an activation difference can be measured by comparing activation of the various receptors by any known method, for example using an in vitro receptor binding assay, such as those produced by NovaScreen Biosciences Corporation (Hanover, Md.), or by the methods disclosed in WO 02/44176 (α4β2 tested), U.S. Pat. No. 6,407,095 (peripheral nicotinic receptor of the ganglion type), U.S. Published Application No. 2002/0086871 (binding of labeled ligand to membranes prepared from GH₄Cl cells transfected with the receptor if interest), U.S. Published Application No. 2002/0086871 (α1 and α4), and WO 97/30998. References which describe methods of determining agonists that are selective for α7 receptors include: U.S. Pat. No. 5,977,144 (Table 1), WO 02/057275 (p 41-42) and Holladay, et al., J. Med. Chem. 40:4169-4194 (1997). The teachings of all of these references are incorporated herein by reference. Assays for other nicotinic receptor subtypes are known to the skill artisan.

In one method, the binding or activity (current responses) of Xenopus oocytes expressing either the α7 receptor subtype or another receptor subtype (e.g., α4β2) is measured after administration of an agonist Agonists that result in greater activation of the α7 receptor subtype are determined to be α7 selective agonists. Using any of the above methods or an equivalent method, it is preferred that the selective α7 agonist is at least two-fold, more preferably at least five-fold, even more preferably at least 10-fold, and most preferably at least 50-fold more able to activate the α7 receptor than at least one other nicotinic receptor.

An agonist is specific for C7 if that agonist activates the α7 receptor to a much greater degree (i.e., at least 10-fold, preferably at least 20-fold, more preferably at least 50-fold) than any other nicotinic receptor. Most preferably, the specific agonist will not activate another nicotinic receptor to any measurable degree (i.e., significant at P=0.05 vs. untreated receptor in a well-controlled comparison). Nonlimiting examples of specific C7 agonists are DMXB-A (Compound (V)), trans-3-(2-methoxy-cinnamylidene)anabaseine and trans-3-(4-methoxycinnamylidene)anabaseine.

In one embodiment, the nicotinic receptor agonist that is used in the combination therapy compositions and methods of the invention is a compound of formula I:

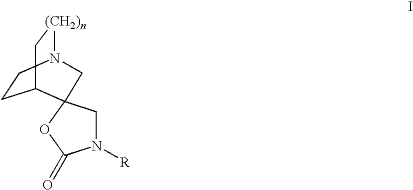

wherein, R represents hydrogen or methyl, and

n represents 0 or 1;

or a pharmaceutically acceptable salt thereof. In particularly preferred embodiments the nicotinic receptor agonist is (−)-spiro[1-azabicyclo[2.2.2]octane-3,5′-oxazolidin-2′-one] (Compound (VII)). Methods of preparation of compounds of formula I are described in U.S. Pat. No. 5,902,814, the teachings of which are incorporated herein by reference in their entirety.

In other embodiments the nicotinic receptor agonist is a compound of formula II:

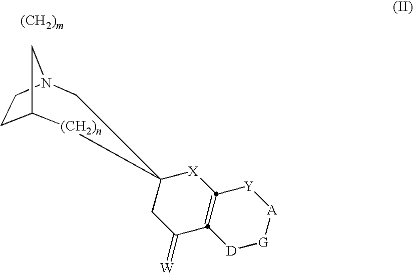

wherein:

m is 1 or 2;

n is 0 or 1;

Y is CH, N or NO;

X is oxygen or sulfur;

W is oxygen, H₂ or F₂;

A is N or C(R²);

G is N or C(R³);

D is N or C(R⁴);

with the proviso that no more than one of A, G and D is nitrogen but at least one of Y, A, G and D is nitrogen or NO;

R¹ is hydrogen or C₁-C₄ alkyl;

R², R³ and R⁴ are independently hydrogen, halogen, C₁-C₄ alkyl, C₂-C₄ alkenyl, C₂-C₄ alkynyl, aryl, heteroaryl, OH, OC₁-C₄ alkyl, CO₂R₁, —CN, —NO₂, —NR₅R⁶, —CF₃ or —OSO₂CF₃, or R² and R³, R³ and R⁴, respectively, may together form another six membered aromatic or beteroaro'matic ring sharing A and G, or G and D, respectively, containing between zero and two nitrogen atoms, and substituted with one to two of the following substitutents: independently hydrogen, halogen, C₁-C₄ alkyl, C₂-C₄ alkenyl, C₂-C₄ alkynyl, aryl, heteroaryl, OH, OC₁-C₄ alkyl, CO₂R′, —CN, —NO₂, —NR⁵R⁶, —CF₃ or —OSO₂CF₃;

R⁵ and R⁶ are independently hydrogen, C₁-C₄ alkyl, C(O)R⁷, C(O)NHR⁸, C(O)OR⁹, SO₂R¹⁰ or may together be (CH₂)ⱼQ(CH₂)ₖ where Q is O, S, NR¹¹,

or a bond;

j is 2 to 7;

k is 0 to 2;

R⁷, R⁸, R⁹, R¹⁰ and R¹¹ are independently C₁-C₄ alkyl, aryl, or heteroaryl, or an enantiomer thereof,

or a pharmaceutically acceptable salt thereof. In preferred embodiments, a nicotinic receptor agonist is a compound of formula II wherein m is 1; n is 0; p is 0; x is oxygen; A is C(R²); G is C(R³); and D is C(R⁴). In a particularly preferred embodiment the nicotinic receptor agonist is (R)-(−)-5′-phenylspiro[1-azabicyclo[2.2.2]octane-3,2′-(3′H)-furo[2,3-b]pyridine]. Methods of preparation of compounds of formula II are described in U.S. Pat. No. 6,110,914, the teachings of which are incorporated herein by reference in their entirety.

In additional embodiments the nicotinic receptor agonist is a compound of formula III:

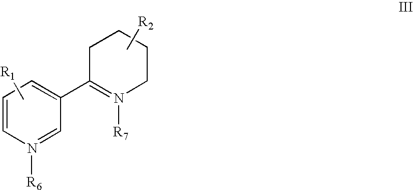

wherein R₁ is hydrogen, R₆ and R₇ are hydrogen or C₁-C₄ alkyl or are absent; and R₂ is selected from a group of

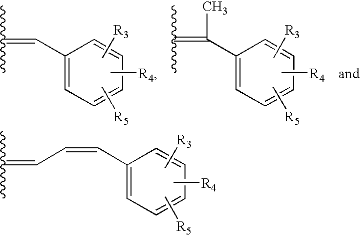

wherein, R₃,

R₄ and R₅ are selected from the group consisting of hydrogen, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, amido having 1 to 4 carbons in the acyl, cyano, and N,N-dialkylamino having 1 to 4 carbons in each of the alkyls, halo, hydroxyl or nitro.

In a preferred embodiment, an agonist is a compound of formula III, wherein R₂ is attached to the 3-position of the tetrahydropyridine ring, and further wherein R₃, which is attached to the 4- or the 2-position of the phenyl ring, is selected from the group consisting of amino, hydroxyl, chloro, cyano, dimethylamino, methyl, methoxy, acetylamino, acetoxy, and nitro. In one particularly preferred embodiment, the agonist is a compound of formula III, wherein R₃ is hydroxyl, and wherein R₁, R₄, and R₅ are hydrogen. In another particularly preferred embodiment, the agonist is a compound of formula III, wherein R₃ is acetylamino and wherein R₁, R₄, and R₅ are hydrogen. In another particularly preferred embodiment, the agonist is a compound of formula III, wherein R₃ is acetoxy and wherein R₁, R₄, and R₅ are hydrogen. In another particularly preferred embodiment, the agonist is a compound of formula III, wherein R₃ is methoxy, and wherein R₁, R₄, and R₅ are hydrogen. In yet another particularly preferred embodiment, the agonist is a compound of formula III, wherein R₃ is methoxy and wherein R₁ and R₄ are hydrogen, and further wherein R₃ is attached to the 2-position of the phenyl ring, and R₅, which is attached to the 4-position of the phenyl ring, is methoxy or hydroxy. In particularly preferred embodiments the nicotinic receptor agonist is selected from the group consisting of 3-2,4-dimethoxybenzylidine anabaseine (DMXB-A; Compound (V)), 3-(4-hydroxybenzylidene)anabaseine, 3-(4-methoxybenzylidene)anabaseine, 3-(4-aminobenzylidene)anabaseine, 3-(4-hydroxy-2-methoxybenzylidene)anabaseine (Compound (VI)), 3-(4-methoxy-2-hydroxybenzylidene)anabaseine, trans-3-cinnamylidene anabaseine, trans-3-(2-methoxy-cinnamylidene)anabaseine and trans-3-(4-methoxycinnamylidene)anabaseine. Methods of preparation of compounds of formula III are described in U.S. Pat. No. 5,977,144, the teachings of which are incorporated herein by reference in their entirety.

In further embodiments the nicotinic receptor agonist is a compound of formula IV:

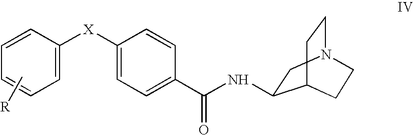

wherein X is O or S; and

R is selected from the group consisting of H, OR₁, NHC(O)R₁, and a halogen, wherein R₁ is a hydrogen or a C₁-C₄ alkyl. In a particularly preferred embodiment the nicotinic receptor agonist is selected from a group consisting of N-[(3R)-1-azabicyclo[2.2.2]oct-3-yl]4-(4-hydroxyphenoxy)benzamide, N-[(3R)-1-azabicyclo[2.2.2]oct-3-yl]4-(4-acetamidophenoxy)benzamide, N-[(3R)-1-azabicyclo[2.2.2]oct-3-yl]4-(phenylsulfanyl)benzamide, and N-[(3R)-1-azabicyclo[2.2.2]oct-3-yl]4-(3-chlorophenylsulphonyl)benzamide. Methods of preparation of compounds of formula IV have are described in PCT Patent Application Publication WO 01/85727, the teachings of which are incorporated herein by reference in their entirety.

In yet other embodiments, the nicotinic receptor agonist is (1-azabicyclo[2.2.2]oct-3-yl)-carbamic acid 1-(2-fluorophenyl)-ethyl ester. Methods of preparation of this compound have been described in U.S. Patent Application Publication 2002/0040035, the teachings of which are incorporated herein by reference in their entirety.

In one embodiment, the nicotinic receptor agonist is selected from the group consisting of 3-2,4-dime oxybenzylidine anabaseine (DMXB-A; Compound V), 3-(4-hydroxybenzylidene)anabaseine, 3-(4-methoxybenzylidene)anabaseine, 3-(4-aminobenzylidene)anabaseine and 3-(4-hydroxy-2-methoxybenzylidene)anabaseine (Compound VI). Compounds V and VI are represented by the following formulae:

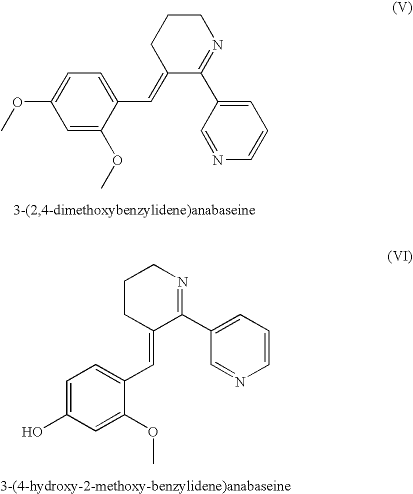

The term “alkyl”, as used herein, unless otherwise indicated, includes saturated monovalent hydrocarbon radicals having straight or branched moieties, typically C₁-C₁₀, preferably C₁-C₆. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The terms “alkoxy”, as used herein, means an “alkyl-O—” group, wherein alkyl is as defined above.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl. “Bicycloalkyl” groups are non-aromatic saturated carbocyclic groups consisting of two rings. Examples of bicycloalkyl groups include, but are not limited to, bicyclo-[2.2.2]-octyl and norbornyl. The term “cycloalkenyl” and “bicycloalkenyl” refer to non-aromatic carbocyclic cycloalkyl and bicycloalkyl moieties as defined above, except comprising of one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl and cyclohexenyl. A non-limiting example of a bicycloalkenyl group is norborenyl. Cycloalkyl, cycloalkenyl, bicycloalkyl, and bicycloalkenyl groups also include groups similar to those described above for each of these respective categories, but which are substituted with one or more oxo moieties. Examples of such groups with oxo moieties include, but are not limited to oxocyclopentyl, oxocyclobutyl, oxocyclopentenyl, and norcamphoryl.

The term “cycloalkoxy”, as used herein, unless otherwise indicated, includes “cycloalkyl-O—” group, wherein cycloalkyl is defined above.

The term “aryl”, as used herein, refers to carbocyclic group. Examples of aryl groups include, but are not limited to phenyl and naphthyl.

The term “heteroaryl”, as used herein, refers to aromatic groups containing one or more heteroatoms (O, S, or N). A heteroaryl group can be monocyclic or polycyclic. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing heteroaryl groups may be C-attached or N-attached (where such is possible). For instance, a group derived from pyrrole may be pyrrol-1-yl (N-attached) or pyrrol-3-yl (C-attached).

In the context of the present invention, a bicyclic carbocyclic group is a bicyclic compound holding carbon only as a ring atom. The ring structure may in particular be aromatic, saturated, or partially saturated. Examples of such compounds include, but are not limited to, indanyl, naphthalenyl, azulenyl.

In the context of the present invention, an amino group may be a primary (—NH₂), secondary (—NH), or tertiary (—NHR_{b}), wherein Rₐ and R_{b} may be any of the alkyl, alkenyl, alkynyl, alkoxy, cycloalkyl, cycloalkoxy, aryl, heteroaryl, and a bicyclic carbocyclic group.

Pharmaceutically acceptable salts of the compounds disclosed herein can be used to practice the present invention. As used herein, a “pharmaceutically acceptable salt” of the disclosed compound is an ionic bond-containing product of reacting a compound of the invention with either an acid or a base, suitable for administering to a subject. For example, an acid salt of a compound containing an amine or other basic group can be obtained by reacting the compound with a suitable organic or inorganic acid, such as hydrogen chloride, hydrogen bromide, acetic acid, perchloric acid and the like. Other examples of such salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. Salts can also be formed with suitable organic bases when the compound comprises an acid functional group such as —COOH or —SO₃H. Such bases suitable for the formation of a pharmaceutically acceptable base addition salts with compounds of the present invention include organic bases that are nontoxic and strong enough to react with the acid functional group. Such organic bases are well known in the art and include amino acids such as arginine and lysine, mono-, di-, and triethanolamine, choline, mono-, di-, and trialkylamine, such as methylamine, dimethylamine, and trimethylamine, guanidine, N-benzylphenethylamine, N-methylglucosamine, N-methylpiperazine, morpholine, ethylenediamine, tris(hydroxymethyl)aminomethane and the like.

In still other embodiments, the α7 agonist used in the combination therapy compositions and methods of the invention is an antibody that is a selective agonist (most preferably a specific agonist) of the α7 nicotinic receptor. As described herein, the antibodies can be polyclonal or monoclonal; may be from any of a number of human, non-human eukaryotic, cellular, fungal or bacterial sources; may be encoded by genomic or vector-borne coding sequences; and may be elicited against native or recombinant α7 or fragments thereof with or without the use of adjuvants, all according to a variety of methods and procedures described herein and/or well-known in the art for generating and producing antibodies. Other examples of such useful antibodies include but are not limited to chimeric, single-chain, and various human or humanized types of antibodies, as well as various fragments thereof, such as described herein.

As described herein, an antibody that is a selective agonist (most preferably a specific agonist) of the α7 nicotinic receptor can be generated by immunizing a suitable laboratory animal with the α7 receptor or a fragment thereof, and isolating the antibodies elicited by the immunization that bind α7. Immunization and isolation procedures are well known to one of ordinary skill in the art. Antibodies that are agonists can be identified using any suitable procedure, for example, by combining the isolated antibodies with a macrophage that has been stimulated to release a proinflammatory cytokine, or any other suitable method for assessing α7 receptor activity. Inhibition of cytokine release is indicative of the agonist activity. Selectivity for α7 can be assessed by screening for activity against at least one other nicotinic or cholinergic receptor, as previously described. Antibodies that are found to be selective agonists for the α7 receptor may be further evaluated for their efficacy in treating one or more of the inflammatory diseases described herein, e.g., additional in vitro tests or in vivo testing in animal models. The present invention also includes α7 selective antibody agonists identified by this method.

Agents that Inhibit HMGB-TLR2 Binding, HMGB-TLR4-Binding, and/or HMGB-RAGE Binding

It has been shown that HMGB polypeptides (e.g., HMGB1) bind Toll-like receptor 2 (TLR2) and that inhibition of the interaction between HMGB and TLR2 can decrease or prevent inflammation (U.S. Published Application No. 20040053841; the entire teachings of which are incorporated herein by reference). Therefore, agents that bind to HMGB and inhibit interaction between HMGB and TLR2 (e.g., antibodies to HMGB, antibodies to HMGB B boxes (as described herein), HMGB small molecule inhibitors), as well as agents that bind to TLR2 and inhibit interaction between HMGB and TLR2 (e.g., antibodies to TLR2, TLR2 small molecule antagonists, soluble TLR2) are encompassed by the invention.

In one embodiment, the combination therapy composition comprises an agent that binds to TLR2 and inhibits interaction between HMGB and TLR2. Such agents include, e.g., an antibody or antigen-binding fragment that binds TLR2, a mutant of a natural ligand, a peptidomimetic, a competitive inhibitor of ligand binding). In one embodiment, the agent is a ligand that binds to TLR2 with greater affinity than HMGB binds to TLR2. Preferably the agent that binds to TLR2, thereby inhibiting binding by HMGB, does not significantly initiate or increase an inflammatory response, and/or does not significantly initiate or increase the release of a proinflammatory cytokine from a cell.

Examples of ligands that are known to bind TLR2 include heat shock protein 60, surfactant protein-A, monophosphoryl lipid A (Botler et al., Infect. Immun. 71(5). 2498-2507 (2003)), muramyl dipeptide (Beutler et al., Blood Cells Mol. Dis. 27(4):728-730 (2001)), yeast-particle zymosan, GPI anchor from Trypanosoma cruzi, Listeria inonocytogenes, Bacillus, lipoteichoic acid, peptidoglycan, and lipopeptides from Streptococcus species, heat killed Mycobacteriua tuberculosis, Mycobactelia aviutn lipopeptide, lipoarabinomannan, mannosylated phosphatidylinositol, Borrelia burgdorferi, Treponema pallidum, Treponema maltophilum (lipopeptides, glycolipids, outer surface protein A), and MALP-2 lipopeptides from Mycoplasma fermentans. Therefore, these molecules, as well as portions of these molecules that bind TLR2 can be used to inhibit the interaction between HMGB and TLR2 and can be used in the combination therapy compositions and methods of the invention.

In another embodiment, the combination therapy composition comprises an agent that binds to HMGB, and prevents HMGB from binding to TLR2. Such an agent can be, for example, a soluble form of recombinant TLR2 (sTLR2) (i.e., TLR2 lacking the intracellular and transmembrane domains, as described, for example, by Iwaki et al., J. Biol. Chem. 277(27):24315-24320 (2002)), an anti-HMGB antibody or antigen-binding fragment (as described herein), or a non-HMGB antibody molecule (e.g., a protein, peptide, or small molecule antagonist) that binds HMGB and prevents it from binding to TLR2. The sTLR2 molecule can contain the extracellular domain (for example, amino acids 1-587 of the TLR2 amino acid sequence (e.g., GenBank Accession Number AAC34133). The sTLR molecule can also be modified with one of more amino acid substitutions and/or post-translational modifications provided such sTLR2 molecules have HMGB binding activity, which can be assessed using methods known in the art. Such sTLR2 molecules can be made, for example, using recombinant techniques. Preferably the sTLR2 has at least 70%, 75%, 80%, 85%, 90%, or 95% to amino acids 1-587 of GenBank Accession Number AAC34133. In another embodiment, the inhibitor is an agent that bind TLR2 at a site different than the HMGB binding site and blocks binding by HMGB (e.g., by causing a conformation change in the TLR2 protein or otherwise altering the binding site for HMGB). In another embodiment, the combination therapy composition comprises a dominant negative mutant protein of TLR2 and a caspase inhibitor.

It has also been shown that receptor signal transduction of HMGB1 occurs in part through Toll-like receptor 4 (TLR4). Park, J. S. et al., J. Biol. Chem. 279(9):7370-77 (2004). Therefore, agents that bind to TLR4 and inhibit HMGB1 binding and/or signaling and/or bind to HMGB and inhibit TLR4-mediated binding and/or signaling are encompassed by the invention. Such agents include, e.g., antibodies to TLR4, TLR4 small molecule antagonists, soluble TLR4, dominant negative mutants of TLR4, mutants of a natural ligand of TLR4, peptidomimetics and competitive inhibitors of ligand binding to TLR4.

In one embodiment, the combination therapy composition comprises a soluble TLR4 and a caspase inhibitor. It has been shown in mice that there is an alternatively spliced TLR4 mRNA (mTLR4), which expresses a partially secreted 20 kDa protein (soluble mTLR4; smTLR4) that inhibits LPS-mediated TNF-α production and NF-κB activation. Iwami, K−1 et al., J. Immunol. 165:6682-6686 (2001); the entire teachings of which are incorporated herein by reference. In another embodiment, the combination therapy composition comprises an antibody that binds TLR4 or an antigen-binding fragment thereof and a caspase inhibitor. Antibodies that bind TLR4 are known in the art. See, e.g., Tabeta, K. et al., Infect Immun. 68(6):3731-3735 (2000); Rabbit anti-TLR-4 (Catalog No. 36-3700; Zymed Laboratories, Inc., San Francisco, Calif.).

It has been shown that HMGB polypeptides bind RAGE and that receptor signal transduction occurs in part through the receptor for advanced glycation end-products (RAGE). Andersson U. et al., Scand. J. Infect. Dis. 35(9):577-84 (2003); Park, J. S. et al., J. Biol. Chem. 279(9):7370-77 (2004). It has further been shown that inhibition of the interaction between HMGB and RAGE can decrease or prevent downstream signaling and cellular activation (Schmidt, A. M. et al., J. Clin. Invest. 108(7):949-955 (2001); Park, J. S. et al., J. Biol. Chem. 279(9):7370-77 (2004). Therefore, agents that bind to HMGB and inhibit interaction between HMGB and RAGE (e.g., antibodies to HMGB, antibodies to HMGB B boxes (as described herein), HMGB small molecule antagonists, as well as agents that bind to RAGE and inhibit interaction between HMGB and RAGE (e.g., antibodies to RAGE, RAGE small molecule antagonists (e.g., as taught in PCT Publication Nos. WO 01/99210, WO 02/069965 and WO 03/075921 and U.S. Published Application No. US 2002/0193432A1)), soluble RAGE (sRAGE; e.g., as taught in Schmidt, A. M. et al., J. Clin. Invest. 108(7):949-955 (2001), U.S. Application No. 2002/0122799 and PCT Publication No. WO 00/20621), RAGE dominant negative mutants (as taught in Schmidt, A. M. et al., J. Clin. Invest. 108(7):949-955 (2001)) are encompassed by the invention.

In one embodiment, the combination therapy composition comprises an agent that binds to RAGE and inhibits interaction between HMGB and RAGE. Such agents include, e.g., an antibody or antigen-binding fragment that binds RAGE, a mutant of a natural ligand, a peptidomimaetic, a competitive inhibitor of ligand binding). In one embodiment, the agent is a ligand that binds to RAGE with greater affinity than HMGB binds to RAGE. Preferably the agent that binds to RAGE, thereby inhibiting binding by HMGB, does not significantly initiate or increase an inflammatory response, and/or does not significantly initiate or increase the release of a proinflammatory cytokine from a cell.

Examples of ligands other than HMGB1 that are known to bind RAGE include: AGEs (advanced glycation endproducts, S100/calgranus and i-sheet fibrils. Schmidt, A. M. et al., J. Clin. Invest. 108(7):949-955 (2001)). Therefore, these molecules, as well as portions of these molecules that bind RAGE can be used to inhibit the interaction between HMGB and RAGE and can be used in the combination therapy compositions and methods of the invention.

In another embodiment, the combination therapy composition comprises an agent that binds to HMGB, and prevents HMGB from binding to RAGE. Such an agent can be, for example, a soluble truncated form of RAGE (sRAGE) (i.e., RAGE lacking its intracellular and transmembrane domains, as described, for example, by Schmidt, A. M. et al., J. Clin. Invest. 108(7):949-955 (2001), U.S. Application No. 2002/0122799 and PCT Publication No. WO 00/20621), an anti-HMGB antibody or antigen-binding fragment (as described herein), or a non-HMGB antibody molecule (e.g., a protein, peptide, or non-peptidic small molecule) that binds HMGB and prevents it from binding to RAGE. The sRAGE molecule can be modified with one of more amino acid substitutions and/or post-translational modifications provided such sRAGE molecules have HMGB binding activity, which can be assessed using methods known in the art. Such sRAGE molecules can be made, for example, using recombinant techniques. Preferably the sRAGE has at least 70%, 75%, 80%, 85%, 90%, or 95% to amino acids 1-587 of GenBank Accession Number AAC34133. In another embodiment, the inhibitor is an agent that bind RAGE at a site different than the HMGB binding site and blocks binding by HMGB (e.g., by causing a conformation change in the RAGE protein or otherwise altering the binding site for HMGB). In another embodiment, the combination therapy composition comprises a dominant negative mutant protein of RAGE and a caspase inhibitor. Dominant negative mutant RAGE proteins, which are capable of binding to RAGE but suppress RAGE-mediated signaling are known in the art, e.g., as described by Schmidt, A. M. et al., J. Clin. Invest. 108(7):949-955 (2001)).

In a particular embodiment, the inhibitor of HMGB receptor binding is not an anti-TLR2 antibody or antigen-binding fragment thereof. In another embodiment, the inhibitor of HMGB receptor binding is not an antibody that binds HMGB1 (an anti-HMGB1 antibody) or an antigen-binding fragment thereof. In yet another embodiment, the inhibitor of HMGB receptor binding is not an antibody that binds HMGB (an anti-HMGB antibody) or an antigen-binding fragment thereof. In another embodiment, the inhibitor is not soluble RAGE (i.e., a portion of the RAGE receptor that binds HMGB1). In another embodiment, the inhibitor is non-microbial (i.e., is not a microbe, derived from a microbe, or secreted or released from a microbe). In still another embodiment, the inhibitor is a mammalian inhibitor (i.e., is a molecule that naturally exists in a mammal, is derived from a molecule that naturally exists in a mammal, or is secreted or released from a mammalian cell), for example, a human inhibitor.

In a particular embodiment, the inhibitor is a small molecule inhibitor (i.e., having a molecular weight of 1000 or less, 500 or less, 250 or less or 100 or less). In another embodiment the inhibitor is a short peptide, having, for example, 50 or fewer amino acids, 30 or fewer amino acids, 25 or fewer amino acids, 20 or fewer amino acids, 10 or fewer amino acids, or 5 or fewer amino acids.

As described herein, inhibitors of HMGB receptor binding and/or signaling also include, e.g., antisense nucleic acids and small double-stranded interfering RNA (RNA interference (RNAi)) that target HMGB, TLR2, TLR4 and/or RAGE. Antisense nucleic acids and RNAi can be used to decrease expression of a target molecule, e.g., HMGB, TLR2, TLR4, RAGE, as is known in the art.

Production and delivery of antisense nucleic acids and RNAi is known in the art (e.g., as taught in PCT Publication WO 2004/016229). In one embodiment, small double-stranded interfering RNA (RNA interference (RNAi)) can be used (e.g., RNAi that targets HMGB, TLR2, TLR4 and/or RAGE) in the compositions and methods of the invention. RNAi is a post-transcription process, in which double-stranded RNA is introduced, and sequence-specific gene silencing results, though catalytic degradation of the targeted mRNA. See, e.g., Elbashir, S M. et al., Nature 411:494-498 (2001); Lee, N. S., Nature Biotech. 19:500-505 (2002); Lee, S-K. et al., Nature Medicine 8(7):681-686 (2002) the entire teachings of these references are incorporated herein by reference.

RNAi is used routinely to investigate gene function in a high throughput fashion or to modulate gene expression in human diseases (Chi et al., Proc. Natl Acad. Sci. U.S.A. 10(11):6343-6346 (2003)). Introduction of long double stranded

RNA leads to sequence-specific degradation of homologous gene transcripts. The long double stranded RNA is metabolized to small 21-23 nucleotide siRNA (small interfering RNA). The siRNA then binds to protein complex RISC(RNA-induced silencing complex) with dual function helicase. The helicase has RNAase activity and is able to unwind the RNA. The unwound siRNA allows an antisense strand to bind to a target. This results in sequence dependent degradation of cognate mRNA. Aside from endogenous RNAi, exogenous RNAi, chemically synthesized or recombinantly produced RNAi can also be used in the compositions and methods of the invention

### Caspase Inhibitors

As described herein, the combination therapy compositions of the invention include a caspase inhibitor. Caspases are a family of cysteine protease enzymes that are key mediators in the signaling pathways for apoptosis (Thornberry, Chem. Biol., 5:R97-R103 (1998)). These signaling pathways vary depending on cell type and stimulus, but all apoptosis pathways appear to converge at a common effector pathway leading to proteolysis of key proteins. Caspases are involved in both the effector phase of the signaling pathway and further upstream, at its initiation. The upstream caspases involved in initiation events become activated, and in turn activate other caspases that are involved in later phases of apoptosis.

The caspases have been grouped according to their known functions. The first subfamily consists of caspase-1 (also known as interleukin converting enzyme or “ICE”), caspase-4, and caspase-5. These caspases are involved in pro-inflammatory cytokine processing and play an important role in inflammation. Caspase-1, the most studied enzyme of this class, activates the IL-1β precursor by proteolytic cleavage and, therefore, plays a key role in the inflammatory response. Caspase-1 is also involved in the processing of interferon gamma inducing factor (IGIF or IL-18), which stimulates the production of interferon gamma, a key immunoregulator that modulates antigen presentation, T-cell activation and cell adhesion.

The remaining caspases make up the second and third subfamilies and are of central importance in the intracellular signaling pathways leading to apoptosis. One subfamily consists of the enzymes involved in initiating events in the apoptotic pathway, including transduction of signals from the plasma membrane. Members of this subfamily include caspases-2, 8, 9 and 10. The other subfamily, consisting of the effector caspases-3, 6 and 7, are involved in the final downstream cleavage events that result in the death of the cell by apoptosis. Caspases involved in the upstream signal transduction activate the downstream caspases, which then disable DNA repair mechanisms, fragment DNA, dismantle the cell cytoskeleton and finally fragment the cell.

Caspases possess highly conserved catalytic and substrate-recognition motifs. Knowledge of these conserved motifs has provided a basis for the design of compounds that inhibit one or more caspase enzymes. Several general and specific caspase inhibitors are known in the art and have been successfully used to inhibit caspase activity.

General caspase inhibitors that are suitable for compositions of the present invention include, but are not limited to, the modified peptide compounds carbobenzoxyl-valyl-alanyl-aspartyl-[O-methyl]-fluoromethylketone (Z-VAD-FMK), Q-VD-OPH, BOC-D-FMK and acetyl-Ile-Gln-Ala-Cys-Arg-Gly-OH (Ac-IQACRG) (SEQ ID NO:46) (Calbiochem, San Diego, Calif.; Catalog No. 235416-SMG), which bind to the catalytic site of caspase proteases. Other suitable general caspase inhibitors include, among others, proteins belonging to the highly-conserved inhibitor of apoptosis (IAP) family of proteins. These proteins contain one or more characteristic BIR domains (Yang et al., Cell Res. 10(3)-169-77 (2000)), and are capable of binding to and preventing activation of multiple caspases. Examples of LAP proteins include, but are not limited to, naturally-occurring and recombinant forms of cIAP1, cIAP2, XIAP, NMAP, survivin, Cp-LAP and Op-LAP. In addition, the present invention encompasses compositions comprising one or more other general caspase inhibitors, such as the compound M-920 (Hotchkiss et al., Nature Immunology 1(6): 497-501 (2000)), carbobenzoxyl-valyl-lysyl(biotin)-aspartyl(O-methyl)-fluoromethylketone (Z-VKD-FMK), BD™ ApoBlock Caspase Inhibitor (BD Biosciences Pharmingen, Catalog No. 552892), discoharbdin compounds (see, e.g., U.S. Pat. No. 6,057,333) and the compound 2,2′-Methylenebis(1,3-cyclohexanedione) (Calbiochem, San Diego, Calif.; Catalog No. M50054).

Caspases have stringent requirements for at least four amino acid residues (referred to as P4-P1) on the N-terminal side of the cleavage site. The P4 residue, which is located immediately next to the cleavage site, appears to be critical for substrate recognition and specificity. Therefore, caspases recognize particular tetrapeptide sequences and, as a consequence, these sequences have formed the basis for the design of specific peptide inhibitors. Such peptidomimetic compounds tend to be specific for a particular group of caspases that share similar substrate recognition requirements. For example, Caspases 1, 4, and 5 prefer hydrophobic aromatic amino acids at position 4 on the N-terminal side of the cleavage site. In contrast, Caspases 2, 3 and 7 recognize aspartyl residues at both positions 1 and 4 (i.e., Asp-X-X-Asp) on the N-terminal side of the cleavage site, but prefer a sequence of AspGlu-X-Asp. A third group, which includes caspases 6, 8, 9 and 10, tolerates many amino acids in the primary recognition sequence, but prefers residues with branched aliphatic side chains, such as valine and leucine, at position 4.

It is possible to generate reversible or irreversible inhibitors of caspase activation by coupling caspase specific peptides to certain compounds. For example, tetrapeptide aldehydes (CHO), nitriles and ketones are potent reversible inhibitors of caspase proteases, while compounds that form thiomethylketone adducts with the active site cysteine (e.g., peptide (acyloxy)methylketones) are potent irreversible inhibitors. In addition, fluoromethyl ketone (FMK)-derivatized peptides act as effective irreversible inhibitors with no added cytotoxic effects. Inhibitors synthesized with a benzyloxycarbonyl/carbobenzoxyl group (also known as BOC or Z) or acetyl group (Ac) at the N-terminus, and O-methyl side chains (OCH₃) exhibit enhanced cellular permeability, thus facilitating their use for in vitro cell culture, as well as in vivo animal studies. In addition to these modifications, various other peptide modifications can be made without disrupting the inhibitory effects of these molecules on caspases.

Thus, the present invention includes compositions that comprise one or more peptide caspase inhibitors. Known peptide caspase inhibitors that are suitable for the compositions of the present invention include, but are not limited to, the following peptides (including modified derivatives thereof): Caspase-1, 4 and/or 5 inhibitors (e.g., WEHD (SEQ ID NO:47), YVAD (SEQ ID NO:48), IETD (SEQ ID NO:49), AEVD (SEQ ID NO:50), LEED (SEQ ID NO:51), LEVD (SEQ ID NO:52) and YVKD (SEQ ID NO:53)); Caspase-2, 3 and/or 7 inhibitors (e.g., VDVAD (SEQ ID NO:54), LDESD (SEQ ID NO:55), DEVD (SEQ ID NO:56), DMQD (SEQ ID NO:57) and DQTD (SEQ ID NO:58)); and Caspase-6, 8, 9 and/or 10 inhibitors (e.g., VEID (SEQ ID NO:59), IETD (SEQ ID NO:60) and LEHHD (SEQ ID NO:61)). Also included is the caspase-8 peptide inhibitor, Ac-Ala-Ala-Val-Ala-Leu-Leu-Pro-Ala-Val-Leu-Leu-Ala-Leu-Leu-Ala-Pro-Ile-Glu-Thr-Asp-CHO (Ac-AAVALLPAVLLALLALPIETD-CHO (SEQ ID NO:62)) and caspase-1 peptide inhibitors, as disclosed in WO 93/05071, having the formula:

Z-Q₂-Asp-Q₁

wherein Z is an N-terminal protecting group; Q₂ is 0 to 4 amino acids such that the sequence Q₂-Asp corresponds to at least a portion of the sequence Ala-Tyr-Val-His-Asp (AYVHD) (SEQ ID NO:63); and Q, comprises an electronegative leaving group.

In general, the peptidic inhibitors are very potent against some of the caspase enzymes. However, this potency has not always been reflected in cellular models of apoptosis. In addition, peptide inhibitors are typically characterized by undesirable pharmacological properties, such as poor oral absorption, poor stability and rapid metabolism (Plattner and Norbeck, in Drug Discovery Technologies, Clark and Moos, Eds. (Ellis Horwood, Chichester, England (1990)). While a number of caspase inhibitors have been reported, it is not clear whether they possess the appropriate pharmacological properties to be therapeutically useful.

As an alternative, various small molecule caspase inhibitors have been developed to provide effective inhibition of apoptosis in vivo. The compositions of the present invention comprise, but are not limited to, such molecules as gamma-ketoacid dipeptide caspase inhibitors and non-peptidyl caspase inhibitors containing a pyrazinone core. Specific small molecule caspase inhibitors that are suitable for compositions of the present invention include, among others, the caspase-1 inhibitor, Z-Asp-[(2,6-dichlorobenzoyl)oxy]methane (Z-Asp-CH₂-DCB) (Peptides International, Louisville, Ky.; Catalog No. ICE-3174-v), the caspase 3 inhibitor, M-791 (Hotchkiss et al., Nature Immunology 1(6): 497-501 (2000)), and the caspase-3/-7 inhibitor, 5-[(S)-( )-2-(Methoxymethyl)pyrrolidino]sulfonylisatin.

In addition, small molecule caspase inhibitors that are suitable for the present invention include compounds of formula VII (see, e.g., U.S. Pat. No. 6,800,619 B2):

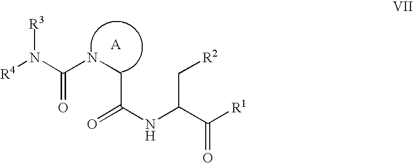

wherein:

Ring A is an optionally substituted piperidine, tetrahydroquinoline or tetrahydroisoquinoline ring;

R¹ is hydrogen, CN, CHN₂, R, or CH₂Y;

R is an optionally substituted group selected from an aliphatic group, an aryl group, or an aralkyl group;

Y is an electronegative leaving group;

R² is CO₂H, CH₂CO₂H, or esters, amides or isosteres thereof; and

R³ is hydrogen, an optionally substituted aryl group, an optionally substituted aralkyl group, or an optionally substituted C₁₋₆ aliphatic group, R⁴ is an optionally substituted group selected from an aryl group or a heterocyclyl group, or R³ and R⁴ taken together with the nitrogen to which they are attached optionally form is a substituted or unsubstituted monocyclic, bicyclic or, tricyclic ring.

Such caspase inhibitor compounds have potent inhibition properties across a range of caspase targets with good efficacy in cellular models of apoptosis. In addition, the compounds are expected to have improved cell penetration and pharmacokinetic properties and, as a consequence of their potency, have improved efficacy against diseases where caspases are implicated.

In one embodiment, the small molecule caspase inhibitor used in the combination therapy compositions and methods of the invention is a compound of formula VIII (see, e.g., U.S. Pat. No. 6,878,743 B2):

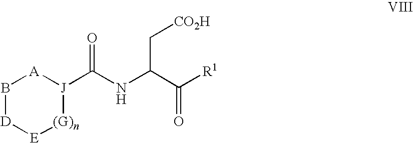

or a pharmaceutically acceptable derivative thereof, wherein

R¹ is H, an aliphatic, heteroaliphatic, aryl, heteroaryl, alkylaryl, alkylheteroaryl, heteroalkylaryl, or heteroalkylheteroaryl moiety;

n is 0 or 1;

A is CR^{A}, C(R^{A})₂, C═O, S, NR^{A}, N(R^{A})₂, or O;

B is CR^{B}, C(R^{B})₂, C═O, S, NR^{B}, N(R^{B})₂, or O;

D is CR^{D}, C(R^{D})₂, C═O, S, NR^{D}, N(R^{D})₂, or O;

E is CR^{E}, C(R^{E})₂, C═O, S, NR^{E}, N(R^{E})₂, or O;

G is CR^{G}, C(R^{G})₂, C═O, S, NR^{G}, N(R^{G})₂, or O;

J is CR^{J};

each of A-B, B-D, D-E, E-G, G-J and A-J are connected by a single or double bond as valency and stability permits;

each occurrence of R^{A}, R^{B}, R^{D}, R^{E}, R^{G} and R^{J} is independently hydrogen, halogen, —OR², —N(R²)₂, —SR², —CN, —COOR², —COR², —CON(R², —SOR², —SO₂R², —SO₂N(R²)₂, —NR² SO₂R², —O(C═O)N(R²)₂, —NR² (C═O)N(R²)₂, —NR² (C═S)N(R²)₂, —NR² SO₂N(R²)₂, or an aliphatic, heteroaliphatic, aryl, heteroaryl, alkylaryl, alkylheteroaryl, heteroalkylaryl, or heteroalkylheteroaryl moiety optionally independently substituted with one or more occurrences of R², wherein each occurrence of R² is independently hydrogen, halogen, —OR³, —N(R³)₂, —SR³, —CN, —COOR³, —COR³, —CON(R³)₂, —SOR³, —SO₂R³, —SO₂N(R³)₂, —NR³SO₂R³, —O(C═O)N(R³)₂, —NR³ (CO)N(R³)₂, —NR³ (C═S)N(R³)₂, —NR³ SO₂N(R³², or an aliphatic, heteroaliphatic, aryl, heteroaryl, alkylaryl, alkylheteroaryl, heteroalkylaryl, or heteroalkylheteroaryl moiety;

wherein each occurrence of R³ is independently hydrogen, or an aliphatic, heteroaliphatic, aryl, heteroaryl, alkylaryl, alkylheteroaryl, heteroalkylaryl, or heteroalkylheteroaryl moiety,

and wherein at least one of R^{B} or R^{D} comprises —SR², —SOR², —SO₂R², —SO₂N(R²)₂, —NR² SO₂R², —N(R²)₂, —(C═O)N(R²)₂, —NR²(C—O)R², —O(C═O)N(R²)₂, —NR² (C═O)N(R²)₂, —NR² (C═S)N(R²)₂, —NR² SO₂N(R²)₂, or is an alkyl or heteroalkyl group substituted with one or more occurrences of R², wherein R² is —SR³, —SOR³, —SO₂R³, —SO₂N(R³, —NR³ SO₂R³, —N(R³)₂, —C═O)N(R³)₂, —NR³ (C═O)R³, —O(C═O)N(R³)₂, —NR³ (C═O)N(R³)₂, —NR³ (C═S)N(R³)₂, —NR³ SO₂N(R³)₂, wherein R³ is an aliphatic, heteroaliphatic, aryl, heteroaryl, alkylaryl, alkylheteroaryl, heteroalkylaryl, or heteroalkylheteroaryl moiety,

whereby each of the foregoing aliphatic, heteroaliphatic, alkyl and heteroalkyl moieties may be independently substituted or unsubstituted, cyclic or acyclic, linear or branched, and each of the foregoing aryl, heteroaryl, alkylaryl, alkylheteroaryl, heteroalkylaryl and heteroalkylheteroaryl moieties may be independently substituted or unsubstituted.

In one embodiment, the small molecule-caspase inhibitor used in the combination therapy compositions and methods of the invention is a compound of formula IX (see, e.g., U.S. Pat. No. 6,699,856 B2):

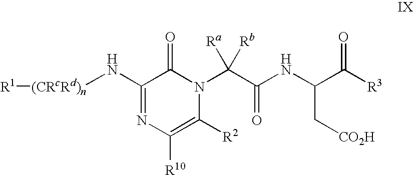

or a pharmaceutically acceptable salt, ester, N-oxide or hydrate thereof wherein:

R¹ is selected from the group consisting of: OH, C₁₋₄ alkyl, HET, Aryl, C₁₋₆ alkoxy, NH₂, NHC₁₋₆ alkyl, N(C₁₋₆ alkyl)₂, C₁₋₆ alkylC(O), C₁₋₆ alkylS(O)_{y}, Aryl-S(O)_{y}, HET-S(O)_{y} wherein y is 0, 1 or 2, Aryl-C(O) and HET-C(O),

the alkyl and alkyl portions of which being optionally substituted with 1-2 members selected from the group consisting of: OH, Aryl¹, HET, halo, NH₂, NHCH₃, N(CH₃)₂, CO₂H, CF₃ and C₁₋₄-acyl;

Aryl represents a C₆₋₁₄ aromatic 1-3 ring system optionally substituted with 1-3 members selected from OH, C₁₋₆ alkyl, OC₁₋₆alkyl, Aryl¹, HET, halo, NH2, NHCH₃, N(CH₃)₂, CF₃, CO₂H and C₁₋₄ acyl;

Aryl¹ represents a C₆₋₁₄ membered aromatic ring system having 1-3 rings and optionally substituted with 1-3 members selected from the group consisting of: OH, HET, halo, NH₂, NHCH₃, N(CH₃)₂, CO₂H and C₁₋₄-acyl;

HET represents a 5 to 15 membered aromatic, partially aromatic or non-aromatic ring system, containing 1-4 heteroatoms selected from O, S and N, and optionally substituted with 1-2 oxo groups and 1-3 groups selected from halo, C₁₋₄ alkyl, C₁₋₄alkoxy, CF₃ and C₁₋₄ acyl;

R^{a} and R^{b} independently represent a member selected from the group consisting of: H, Aryl, C₁₋₆alkyl optionally substituted by 1-3 of halo, 0R⁴, SR⁴ and C₅₋₇ cycloalkyl optionally containing one heteroatom selected from O, S and NR⁵,

or in the alternative, R^{a} and R^{b} are taken in combination and represent a non-aromatic carbocyclic 4-7 membered ring, optionally containing one heteroatom selected from O, S and NR⁵;

R⁴ is selected from the group consisting of: H, C₁₋₅ alkyl, Aryl and Aryl-CIA alkyl optionally substituted with 1-2 groups selected from halo and C₁₋₄ alkyl;

R⁵ is H, C₁₋₄ alkyl or C₁₋₄ acyl;

R^{c} and R^{d} each independently represents a member selected from the group consisting of: H, C₁₋₆ alkyl and Aryl, or in the alternative, R^{c} and R^{d} are taken in combination and represent a non-aromatic carbocyclic ring of 3-7 members, optionally containing one heteroatom selected from O, S and NR⁵;

n is an integer from 0-6 inclusive;

R² represents H, halo or C₁₋₆ alkyl;

R³ represents H, C₁₋₆ alkyl, Aryl, HET, C₁₋₆ alkylSR⁶, C₁₋₆ alkylOR⁶, C₁₋₆ alkylOC(O)R⁷ or C₁₋₆alkylNR⁸R⁹;

R⁶ represents C₁₋₆ alkyl, Aryl, HET or Aryl-C₁₋₆ alkyl, said alkyl and the alkyl portions being optionally substituted with 1-3 members selected from the group consisting of: OH, halo, NH₂, NHCH₃, N(CH₃)₂, CO₂H, CF₃ and C₁₋₄ acyl;

R⁷ represents C₁₋₈ alkyl, Aryl or HET;

R⁸ and R⁹ independently represent H, C₁₋₁₀ alkyl, Aryl, HET, C₆₋₄ alkylN(C₁₋₆ alkyl)₀₋₂, Aryl-C₁₋₆ alkyl, C₁₋₆ alkylOH, or C₁₋₄ alkylOC₁₋₆ alkyl, or R⁸ and R⁹ are taken in combination with the nitrogen atom to which they are attached and represent a 3-10 membered ring system containing 1-4 heteroatoms selected from O, S, N and optionally substituted with 1-2 oxo groups, and 1-3 groups selected from C₁₋₄ alkyl, HET, CO₂R^{c} and C(O)N(R)₂,

said alkyl and alkyl portions being optionally substituted with 1-3 groups selected from halo, C₁₋₃ alkyl, hydroxyC₁₋₃ alkyl, C₁₋₃ alkoxy, C₁₋₃ alkoxyC₁₋₃ alkyl and Aryl¹, and

R¹⁰ represents H, C₁₋₂₀ alkyl, aryl or HU, with aryl and HET as previously described.

In one embodiment, the small molecule caspase inhibitor used in the combination therapy compositions and methods of the invention is a compound of formula X (see, e.g., U.S. Pat. No. 6,525,025 B2):

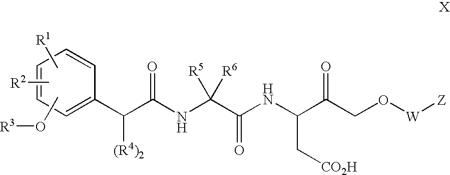

or a pharmaceutically acceptable salt, ester or hydrate thereof, wherein:

W is a bond, —CH₂—; —C(O)— or —C(O)CH₂—;

Z is selected from the group consisting of:
(1) H,
(2) C₁₋₁₁alkyl,
(3) C₃₋₁₁cycloalkyl or a benzofused analog thereof,
(4) phenyl or naphthyl, and
(5) HET¹, wherein HET¹ represents a 5- to 10-membered mono- or bicyclic, aromatic or non-aromatic ring, or a benzofused analog thereof, containing 1-3 heteroatoms selected from O, S and N,

groups (2), (3) and (5) above are optionally substituted with 1-2 oxo groups,

groups (2)-(5) above are further optionally substituted with 1-3 substituents independently selected from the group consisting of:
(a) halo,
(b) nitro,
(c) hydroxy,
(d) C₁₋₄alkyl,
(e) C₁₋₄alkoxy,
(f) C₁₋₄alkylthio,
(g) C₃₋₆cycloalkyl,
(h) phenyl or naphthyl,
(i) phenoxy,
(j) benzyl,
(k) benzyloxy, and
(l) a 5 or 6-membered aromatic or non-aromatic ring containing from 1-3 heteroatoms selected from O, S and N,

groups (d)-(g) above are optionally substituted with oxo and 1-3 substituents independently selected from halo and C₁₋₄alkoxy,

groups (h)-(l) above are optionally substituted with 1-3 substituents independently selected from halo and C₁₋₄alkyl, and

group (4) is further optionally substituted up to its maximum with halo groups;

R¹ and R² are independently selected from the group consisting of:
(1) H,
(2) halo,
(3) hydroxy,
(4) nitro,
(5) cyano,
(6) C₁₋₁₀alkyl, C₃₋₁₀cycloalkyl, C₁₋₁₀alkoxy, —S(O)₀₋₂ C₁₋₁₀alkyl or —NHC₁₋₁₀ alkyl, each optionally substituted with 1-2 oxo or carboxy groups and further optionally substituted with 1-3 substituents independently selected from the group consisting of:
(a) halo,
(b) hydroxy,
(c) cyano,
(d) C₁₋₄ alkoxy,
(e) —NHR⁷, wherein R⁷ is H or C₁₋₅alkyl, said C₁₋₅alkyl optionally substituted with —NHR⁸, wherein R⁸ is C₁₋₅alkyl optionally substituted with oxo and further optionally substituted with a 5- to 10-membered mono- or bicyclic, aromatic or non-aromatic ring, or a benzofused analog thereof, containing 1-3 heteroatoms selected from O, S and N, and optionally substituted with oxo,
(f) —S(O)₀₋₂ C₁₋₄alkyl, and
(g) HET², wherein HET² represents a 5- to 7-membered aromatic or non-aromatic ring containing 1-4 heteroatoms selected from O, S and NR⁷, wherein R⁷ is H or C₁₋₅alkyl, said HET² being optionally substituted with oxo and further optionally substituted with 1-2 substituents independently selected from halo and C₁₋₄alkyl, said C₁₋₄alkyl being optionally substituted with 1-3 halo groups,
(7) phenoxy or —S(O)₀₋₂-phenyl,
(8) benzyloxy or —S(O)₀₋₂-benzyl,
(9) benzoyl,
(10) phenyl or naphthyl,
(11) —O-HET² or —S-HET², said HET² being optionally substituted with oxo and further optionally substituted as defined below, and
(12) HET³, wherein HET is a 5- or 6-membered aromatic or non-aromatic ring, or a benzofused analog thereof, containing from 1 to 4 heteroatoms selected from O, S and N, said HET³ being optionally substituted with oxo and further optionally substituted as defined below,

groups (7)-(12) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups; or R¹ and R² may be taken in combination and represent a fused ring as shown below:

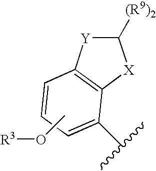

wherein Y and X are independently selected from the group consisting of —C(R¹⁰)₂, —C(R¹⁰)₂C(R¹⁰)₂—, NR¹¹—, —O— and —S—, R³ is as defined below, each R⁹ is independently selected from H and C₁₋₄alkyl, each R¹⁰ is independently selected from H and C₁₋₄alkyl, and R¹¹ is H or C₁₋₄alkyl, or one R⁹ may be joined with either one R¹⁰ or R¹¹ on an adjacent atom to form a double bond;

R³ is C₁₋₁₀alkyl, optionally substituted with 1-2 oxo or carboxy groups and further optionally substituted with 1-3 substituents independently selected from the group consisting of:

(a) halo,

(b) hydroxy,

(c) cyano,

(d) C₁₋₄ alkoxy,

(e) —NHR⁷, wherein R⁷ is H or C₁₋₅alkyl, said C₁₋₅alkyl optionally substituted with —NHR⁸, wherein R⁸ is C₁₋₅alkyl optionally substituted with oxo and further optionally substituted with a 5- to 10-membered mono- or bicyclic, aromatic or non-aromatic ring, or a benzofused analog thereof, containing 1-3 heteroatoms selected from O, S and N, and optionally substituted with oxo,

(f) —S(O)₀₋₂ C₁₋₄alkyl, and

(g) HET², wherein HET² represents a 5- to 7-membered aromatic or non-aromatic ring containing 1-4 heteroatoms selected from O, S and NR⁷, wherein R⁷ is H or C₁₋₅alkyl, said HET² being optionally substituted with oxo and further optionally substituted with 1-2 substituents independently selected from halo or C₁₋₄alkyl, said C₁₋₄alkyl being optionally substituted with 1-3 halo groups,

each R⁴ is independently selected from the group consisting of: H, halo, hydroxy, C₁₋₆alkyl and C₁₋₄alkoxy, said C₁₋₆alkyl and C₁₋₄alkoxy being optionally substituted with oxo and further optionally substituted with 1-3 halo groups; and

R⁵ is selected from the group consisting of: H, phenyl, naphthyl, C₁₋₆alkyl optionally substituted with OR¹² and 1-3 halo groups, and C₅₇cycloalkyl optionally containing one heteroatom selected from O, S and NR¹³,

wherein R¹² is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy, and

R¹³ is H or C₁₋₄alkyl optionally substituted with 1-3 halo groups; and

R⁶ represents H;

or in the alternative, R⁵ and R⁶ are taken in combination and represent a ring of 4-7 members, said ring optionally containing one heteroatom selected from O, S and NR¹³.

Other small molecule inhibitors that are useful for compositions of the present invention include known caspase-1 (ICE) inhibitors (see, e.g., U.S. Pat. No. 6,566,338 B1), as described below:

WO 96/03982 discloses aspartic acid analogs as ICE inhibitors with the formula XI:

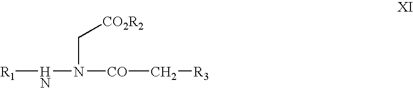

wherein R₂ is H or alkyl; R₃ is a leaving group such as halogen; R₁ is heteroaryl-CO or an amino acid residue.

U.S. Pat. No. 5,585,357 discloses peptidic ketones as ICE inhibitors with the formula XII:

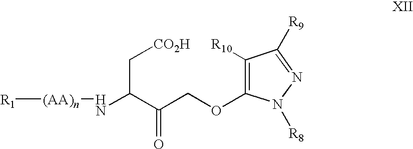

wherein n is 0-2; each AA is independently L-valine or L-alanine; R₁ is selected from the group consisting of N-benzyloxycarbonyl and other groups; R₈, R₉, R₁₀ are each independently hydrogen, lower alkyl and other groups.

Mjalli et al. (Bioorg. Med. Chem. Lett. 3:2689-2692 (1993)) report the preparation of peptide phenylalkyl ketones as reversible inhibitors of ICE, such as compounds having formula XIII:

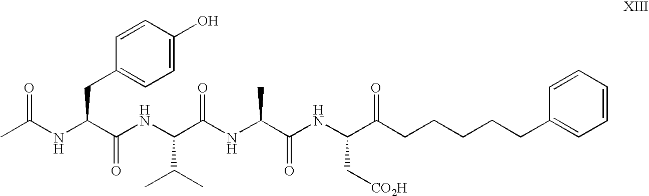

Thornberry et al. (Biochemistry 33:3934-3940 (1994)) report the irreversible inactivation of ICE by peptide acyloxymethyl ketones, such as compounds having formula XIV:

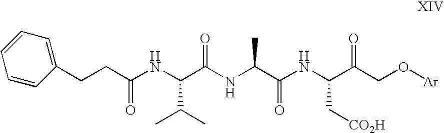

wherein Ar is COPh-2,6-(CF₃)₂, COPh-2,6-(CH₃)₂, Ph-F₅ and other groups.

Dolle et al. (J. Med. Chem. 37:563-564 (1994)) report the preparation of PI aspartate-based peptide α-((2,6-dichlorobenzoyl)oxy)methyl ketones as potent time-dependent inhibitors of ICE, such as formula XV:

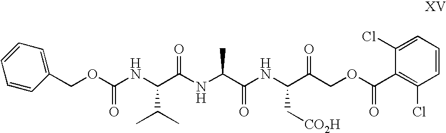

Mjalli et al. (Bioorg. Med. Chem. Lett. 4:1965-1968 (1994)) report the preparation of activated ketones as potent reversible inhibitors of ICE, such as compounds having formula XVI:

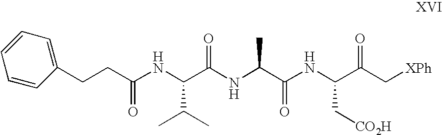

wherein X is NH(CH₂)₂, OCO(CH₂)₂, S(CH₂)₃ and other groups.

Dolle et al. (J. Med. Chem. 37:3863-3866 (1994)) report the preparation of a ((1-phenyl-3-(trifluoromethyl)pyrazol-5-yl)oxy)methyl ketones as irreversible inhibitor of ICE, such as formula XVII:

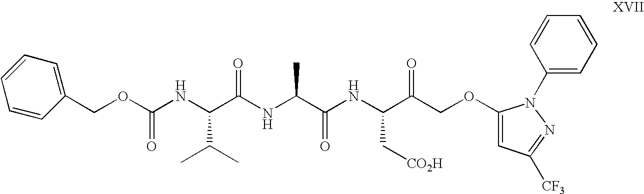

Mjalli et al. (Bioorg. Med. Chem. Lett. 5:1405-1408 (1995)) report inhibition of ICE by N-acyl-Aspartic acid ketones, such as compounds having formula XVIII:

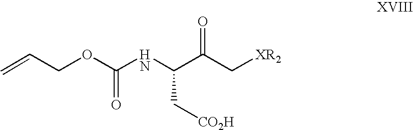

wherein XR₂ is NH(CH₂)₂ Ph, OCO(CH₂)₂ cyclohexyl and other groups.

Mjalli et al. (Bioorg. Med. Chem. Lett. 5:1409-1414 (1995)) report inhibition of ICE by N-acyl-aspartyl aryloxymethyl ketones, such as formula XIX:

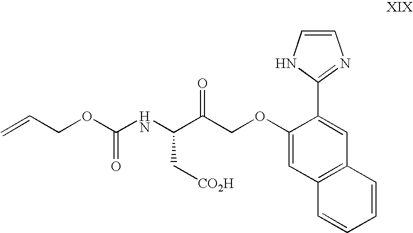

Dolle et al. (J. Med. Chem. 38:220-222 (1995)) report the preparation of aspartyl α-((diphenylphosphinyl)oxy)methyl ketones as irreversible inhibitors of ICE, such as formula XX:

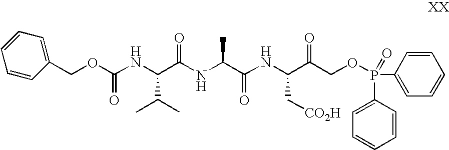

Graybill et al. (Bioorg. Med. Chem. Lett. 7:41-46 (1997)) report the preparation of α-((tetronoyl)oxy)- and α-((tetramoyl)oxy)methyl ketones as inhibitors of ICE, such as formulae XXI and XXII, respectively:

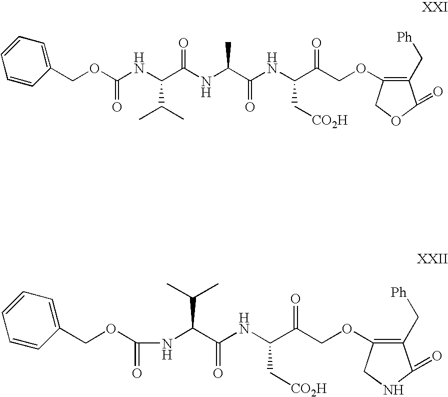

Semple et al. (Bioorg. Med. Chem. Lett. 8:959-964 (1998)) report the preparation of peptidomimetic aminometh-ylene ketones as inhibitors of ICE, such as formula XXIII:

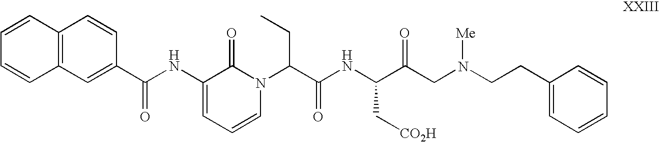

Okamoto et al. (Chem. Pharm. Bull 47:11-21 (1999)) report the preparation of peptide based ICE inhibitors with the P1 carboxyl group converted to an amide, such as formula XXV:

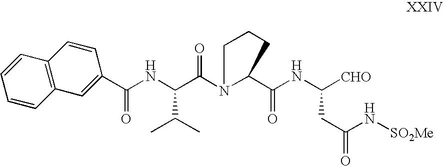

EP618223 discloses inhibitors of ICE as anti-inflammatory agents having the formula:

R-A₁-A₂-X-A₃, wherein

R is a protecting group or optionally substituted benzyloxy;

A₁ is an α-hydroxy or α-amino acid residue or a radical of formula:

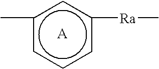

wherein ring A is optionally substituted by hydroxy or C₁₋₄ alkoxy and Rₐ is CO or CS; A₂ is an α-hydroxy or α-amino acid residue or A₁ and A₂ form together a pseudo-dipeptide or a dipeptide mimetic residue; X is a residue derived from Asp; A₃ is —CH₂—X₁—CO—Y₁, —CH₂—O—Y₂, —CH₂—S—Y₃, wherein X₁ is O or S; Y₁, Y₂ or Y₃ is cycloaliphatic residue, and optionally substituted aryl.

WO 99/18781 and U.S. application Ser. No. 09/168,945 disclose dipeptides of formula XXV:

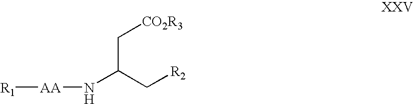

wherein R₁ is an N-terminal protecting group; AA is a residue of any natural or non-natural α-amino acid, β-amino acid, derivatives of an α-amino acid or β-amino acid; R₂ is H or CH₂R₄ where R4 is an electronegative leaving group, and R₃ is alkyl or H, provided that AA is not His, Tyr, Pro or Phe.

Dipeptides of formula XXV are surprisingly potent caspase inhibitors of apoptosis in cell based systems. These compounds are systemically active in vivo and are potent inhibitors of antiFas-induced lethality in a mouse liver apoptosis model and have robust neuroprotective effects in a rat model of ischemic stroke. Exemplary preferred inhibitors of apoptosis include Boc-Ala-Asp-CH₂F, Boc-Val-Asp-CH₂F, Boc-Leu-Asp-CH₂F, Ac-Val-Asp-CH₂F, Ac-Ile-Asp-CH₂F, AG-Met-Asp-CH₂F, Cbz-Val-Asp-CH₂F, Cbz-Ile-Ala-Asp-CH₂F, Cbz-Leu-Asp-CH₂F, Cbz-Ile-Asp-CH₂F, Boc-Ala-Asp(OMe)-CH₂F, Boc-Val-Asp(OMe)-CH₂F, Boc-Leu-Asp(OMe)-CH₂F, Ac-Val-Asp(OMe)-CH₂F, Ac-Dle-Asp(OMe)-CH₂F, Ac-Met-Asp(OMe)-CH₂F, Cbz-Val-Asp(OMe)-CH₂F, Cbz-β-Ala-Asp(OMe)-CH₂F, Cbz-Leu-Asp(OMe)CH₂F and Cbz-Ile-Asp(OMe)-CH₂F.

WO 99/47154 and U.S. application Ser. No. 09/270,735 disclose dipeptides of formula XI:

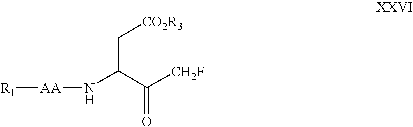

wherein R₁ is an N-terminal protecting group; AA is a residue of a non-natural α-amino acid or β-amino acid; R₂ is an optionally substituted alkyl or H. Exemplary inhibitors of caspases and apoptosis include Boc-Phg-Asp-fnk, Boc-(2-F-Phg)Asp-fmk, Boc-(F₃-Val)-Asp-fmk, Boc-(3-F-Val)Asp-fmk, Ac-Phg-Asp-fmk, Ac-(2-F-Phg)-Asp-fmk, Ac-(F₃-Val)-Asp-fmk, Ac-(3-F-Val)-Asp-fmk, Z-Phg-Asp-fmk, Z-(2-F-Phg)-Asp-fmk, Z-(F₃-Val)-Asp-fin, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(3-Ala)Asp-fmk, Z-(3-F-3-Me-Ala)-Asp-fmk, Z-(3-C-3-F-Ala)-Asp-fmk, Z-(2-Me-Val)Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF₃-Ala)-Asp-fmk, Z-(4-CF₃-Phg)-Asp-fmk, Z-(3-Me₂N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, and Z-(2-Thg)-Asp-fmk; where Z is benzyloxycarbonyl, BOC is tert.-butoxycarbonyl, Ac is acetyl, Phg is phenylglycine, 2-F-Phg is (2-fluorophenyl)glycine, F₃-Val is 4,4,4-trifluorovaline, 3-F-Val is 3-fluoro-valine, 2-Thg is (2-thienyl)glycine, Chg is cyclohexylglycine, 2-Fug is (2-furyl)glycine, 4-F-Phg is (4-fluorophenyl)glycine, 4-Cl-Phg is (4-chlorophenyl)glycine, 3-Thg is (3-thienyl)glycine, 2-Fua is (2-furyl)alanine, 2-Tha is (2-thienyl)alanine, 3-Fua is (3-furyl)alanine, 3-Tha is (3-thienyl)alanine, 3-Cl-Ala is 3-chloroalanine, 3-F-Ala is 3-fluoroalanine, F₃-Ala is 3,3,3-trifluoroalanine, 3-F-3-Me-Ala is 3-fluoro-3-methylalanine, 3-C₁₋₃-F-Ala is 3-chloro-3-fluoroalanine, 2-Me-Val is 2-methylvaline, 2-Me-Ala is 2-methylalanine, 2-i-Pr-β-Ala is 3-amino-2-isopropylpropionic acid, 3-Ph-β-Ala is 3-amino-3-phenylpropionic acid, 3-CN-Ala is 3-cyanoalanine, 1-Nal is 3-(1-naphthyl)-alanine, Cha is cyclohexylalanine, 3-CF₃-Ala is 2-amino-4,4,4-trifluorobutyric acid, 4-CF₃-Phg is 4-trifluoromethylphenylglycine, 3-Me₂N-Ala is 3-dimethylamino-alanine, 2-Abu is 2-aminobutyric acid, Tle is tert-leucine, Cpg is cyclopentylglycine, Cbg is cyclobutylglycine, and Thz is thioproline.

In one embodiment, the caspase inhibitor has formula XXVII:

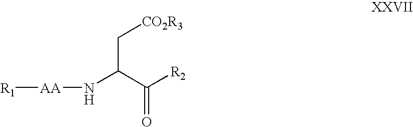

or a pharmaceutically acceptable salt thereof; wherein

R₁ is an N-terminal protecting group;

AA is a residue of any natural or non-natural α-amino acid, β-amino acid, derivatives of an α-amino acid or β-amino acid;

R₂ is H or CH₂R₄ where R₄ is an electronegative leaving group; and

R₃ is alkyl or H.

Examples of such caspase inhibitors include Boc-Ala-Asp-CH₂F, Boc-Val-Asp-CH₂F, Boc-Leu-Asp-CH₂F, Ac-Val-Asp-CH₂F, Ac-Ile-Asp-CH₂F, Ac-Met-Asp-CH₂F, Cbz-Val-Asp-CH₂F, Cbz-#-Ala-Asp-CH₂F, Cbz-Leu-Asp-CH₂F, Cbz-Ile-Asp-CH₂F, Boc-Ala-Asp(OMe)-CH₂F, Boc-Val-Asp(OMe)-CH₂F, Boc-Leu-Asp(OMe)-CH₂F, Ac-Val-Asp(OMe)-CH₂F, Ac-Ile-Asp(OMe)-CH₂F, Ac-Met-Asp(OMe)-CH₂F, Cbz-Val-Asp(OMe)-CH₂F, Cbz-g-Ala-Asp(OMe)-CH₂F, Cbz-Leu-Asp(OMe)CH₂F or Cbz-Ile-Asp(OMe)-CH₂F.

In another embodiment, the caspase inhibitor has the formula XXVIII:

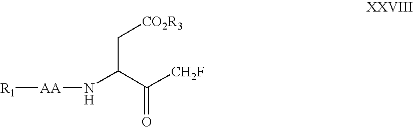

or a pharmaceutically acceptable salt thereof; wherein

R₁ is an N-terminal protecting group;

AA is a residue of a non-natural α-amino acid or β-amino acid; and

R₂ is an optionally substituted alkyl or H.

Examples of such caspase inhibitors include Boc-Phg-Asp-fmk, Boc-(2-F-Phg)-Asp-fmk, Boc-(F₃-Val)-Asp-fmk, Boc-(3-F-Val)-Asp-fmk, Ac-Phg-Asp-fmk, Ac-(2-F-Phg)-Asp-fmk, Ac-(F₃-Val)-Asp-fmk, Ac-(3-F-Val)Asp-fmk, Z-Phg-Asp-fmk Z-(2-F-Phg)-Asp-fmk, Z-(F₃-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-3-Thg)-Asp-fmk, Z-(2-Fua)-Asp-fmk, Z-(2-Tha)-Asp-fmk, Z-3-Fua)-Asp-fmk, Z-(3-Tha)-Asp-fmk, Z-(3-Cl-Ala)-Asp-fmk, Z-(3-F-Ala)-Asp-fmk, Z-(F₃-Ala)-Asp-fmk, Z-(3-F-3-Me-Ala)-Asp-fmk, Z-(3-C₁₋₃-F-Ala)-Asp-fmk, Z-(2-Me-Val)Asp-ink, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-3-CF₃-Ala)Asp-fmk, Z-(4-CF₃-Phg)-Asp-fmk, Z-(3-Me₂N-Ala)-Asp-fmk, Z-(2-Abu)-Asp-ink, Z-Tle-Asp-fmk, Z-Cpg-Asp-fmk, Z-Cbg-Asp-fmk, Z-Thz-Asp-fmk, Z-(3-F-Val)-Asp-fmk, and Z-2-Thg)Asp-fink.

Other caspase inhibitors that can be used in the practice of the invention include without limitation those described in WO93/05071, WO93/09135, WO93/14777, WO 95/26958, WO 95/29672, WO 95/33751, WO 96/03982, WO 96/30395, WO 97/07805, WO 97/08174, WO 97/22618, WO 97/27220, WO 98/11109, WO 98/11129, WO 98/16502, WO 98/16504, WO 98/16505, WO 98/24804, WO 98/24805, EP 519748, EP 547699, EP 618223, EP 623592, EP 623606, EP 628550, EP 644198, U.S. Pat. No. 5,430,128, U.S. Pat. No. 5,434,248, U.S. Pat. No. 5,462,939, U.S. Pat. No. 5,552,400, U.S. Pat. No. 5,565,430, U.S. Pat. No. 5,585,357, U.S. Pat. No. 5,585,486, U.S. Pat. No. 5,622,967, U.S. Pat. No. 5,639,745, U.S. Pat. No. 5,656,627, U.S. Pat. No. 5,670,494, U.S. Pat. No. 5,677,283, U.S. Pat. No. 5,716,929, U.S. Pat. No. 5,739,279, U.S. Pat. No. 5,756,465, U.S. Pat. No. 5,756,466, U.S. Pat. No. 5,798,247, U.S. Pat. No. 5,798,442, U.S. Pat. No. 5,834,514, U.S. Pat. No. 5,843,904, U.S. Pat. No. 5,843,905, U.S. Pat. No. 5,847,135, U.S. Pat. No. 5,866,545, U.S. Pat. No. 5,843,904, U.S. Pat. No. 5,843,905, U.S. Pat. No. 5,847,135, U.S. Pat. No. 5,866,545, U.S. Pat. No. 5,869,519, U.S. Pat. No. 5,874,424, U.S. Pat. No. 5,932,549, Mjalli et al., Bioorg. Med. Chem. Lett. 3:2689-2693 (1993), Mjalli et al., Bioorg. Med. Chem. Lett. 4:1965-1968 (1994), Mjalli et al., Bioorg. Med. Chem. Lett. 5:1405-1408 (1995), Mjalli et al., Bioorg. Med. Chem. Lett. 5:1409-1414 (1995), Thornberry et al., Biochem. 33:3934-3940 (1994), Dolle et al., J. Med. Chem. 37: 563-564 (1994), Dolle et al., J. Med. Chem. 37: 3863-3866 (1994), Dolle et al., J. Med. Chem. 38: 220-222 (1995), Graybill et al., Bioorg. Med. Chem. Lett. 7:41-46 (1997), Semple et al., Bioorg. Med. Chem. Lett. 8:959-964 (1998), and Okamoto et al., Chem. Pharm. Bull. 47:11-21 (1999).

With regard to the caspase inhibitors described herein, useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups. Also contemplated is a trimethylene group substituted on two adjoining positions on the benzene ring of the compounds of the invention.

Optional substituents include one or more alkyl; halo; haloalkyl; cycloalkyl; aryl optionally substituted with one or more lower alkyl, halo, haloalkyl or heteroaryl groups; aryloxy optionally substituted with one or more lower alkyl, halo, haloalkyl or heteroaryl groups; aralkyl; heteroaryl optionally substituted with one or more lower alkyl haloalkyl and aryl groups; heteroaryloxy optionally substituted with one or more lower alkyl, haloalkyl and aryl groups; alkoxy; alkylthio; arylthio; amino; acyloxy; arylacyloxy optionally substituted with one or more lower alkyl, halo alkyl and aryl groups; diphenylphosphinyloxy optionally substituted with one or more lower alkyl, halo or haloalkyl groups; heterocyclo optionally substituted with one or more lower alkyl, haloalkyl and aryl groups; heterocycloalkyloxy optionally substituted with one or more lower alkyl, haloalkyl and aryl groups; partially unsaturated heterocycloalkyl optionally substituted with one or more lower alkyl, haloalkyl and aryl groups; or partially unsaturated heterocycloalkyloxy optionally substituted with one or more lower alkyl, haloalkyl and aryl groups.

Useful aryl groups are C₆₋₁₄ aryl, especially C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as defined above, as well as cycloalkenyl groups, such as cyclopentenyl, cycloheptenyl and cyclooctenyl.

Useful halo or halogen groups include fluorine, chlorine, bromine and iodine.

Useful arylalkyl groups include any of the above-mentioned C₁₋₁₀ alkyl groups substituted by any of the above-mentioned C₆₋₁₄ aryl groups. Useful values include benzyl, phenethyl and naphthylmethyl.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl, chloromethyl, chlorofluoromethyl and trichloromethyl groups.

Useful alkoxy groups include oxygen substituted by one of the C₁₋₁₀ alkyl groups mentioned above.

Useful alkylthio groups include sulphur substituted by one of the C₁₋₁₀ alkyl groups mentioned above. Also included are the sulfoxides and sulfones of such alkylthio groups.

Useful acylamino groups are any C₁₋₆ acyl(alkanoyl) attached to an amino nitrogen, e.g. acetamido, propionamido, butanoylamido, pentanoylamido, hexanoylamido as well as aryl-substituted C₂₄ substituted acyl groups.

Useful acyloxy groups are any C₁₋₆ acyl(alkanoyl) attached to an oxy (—O—) group, e.g. formyloxy, acetoxy, propionoyloxy, butanoyloxy, pentanoyloxy, hexanoyloxy and the like.

Useful arylacyloxy groups include any of the aryl groups mentioned above substituted on any of the acyloxy groups mentioned above, e.g. 2,6-dichlorobenzoyloxy, 2,6-difluorobenzoyloxy and 2,6-di-(trifluoromethyl)-benzoyloxy groups.

Useful amino groups include —NH₂, —NHR₁₁, and —NR₁₁R₁₂, wherein R₁₁ and R₁₂ are C₁₋₁₀ alkyl or cycloalkyl groups as defined above.

Useful saturated or partially saturated heterocyclic groups include tetrahydrofuranyl, pyranyl, piperidinyl, piperizinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

Useful heteroaryl groups include any one of the following: thienyl, benzo[b]thienyl, naphtho[2,3-b]thienyl, thianthrenyl, furyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g. a pyridyl N-oxide, pyrazinyl N-oxide, pyrimidinyl N-oxide and the like.

N-terminal protecting groups include t-butyloxycarbonyl, acetyl and benzyloxycarbonyl.

Amino acids include any of the naturally occurring amino acids such as the L-forms of tyrosine, glycine, phenylalanine, methionine, alanine, serine, isoleucine, leucine, threonine, valine, proline, lysine, histidine, glutamine, glutamic acid, tryptophan, arginine, aspartic acid, asparagine and cysteine. Examples of non-natural amino acids include, without limitation, the enantiomeric and racemic forms of 2-methylvaline, 2-methylalanine, (2-i-propyl)-β-alanine, phenylglycine, 4-methylphenylglycine, 4-isopropylphenylglycine, 3-bromophenylglycine, 4 bromophenylglycine, 4-chlorophenylglycine, 4-methoxyphenylglycine, 4-ethoxyphenylglycine, 4-hydroxyphenylglycine, 3-hydroxyphenylglycine, 3,4-dihydroxyphenylglycine, 3,5-dihydroxyphenylglycine, 2,5-dihydrophenylglycine, 2-fluorophenylglycine, 3-fluorophenylglycine, 4-fluorophenylglycine, 2,3-difluorophenylglycine, 2,4-difluorophenylglycine, 2,5-difluorophenylglycine, 2,6-difluorophenylglycine, 3,4-difluorophenylglycine, 3,5-difluorophenylglycine, 2-(trifluoromethyl)phenylglycine, 3-(trifluoromethyl)phenylglycine, 4-(trifluoromethyl)phenylglycine, 2-(2-thienyl)glycine, 2-(3-thienyl)glycine, 2-(2-furyl)glycine, 3-pyridylglycine, 4-fluorophenylalanine, 4-chlorophenylalanine, 2-bromophenylalanine, 3-bromophenylalanine, 4-bromophenylalanine, 2-naphthylalanine, 3-(2-quinoyl)alanine, 3-(9-anthracenyl)alanine, 2-amino-3-phenylbutanoic acid, 3-chlorophenylalanine, 3-(2-thienyl)alanine, 3-(3-thienyl)alanine, 3-phenylserine, 3-(2-pyridyl)serine, 3-(3-pyridyl)serine, 3-(4-pyridyl)serine, 3-(2-thienyl)serine, 3-(2-furyl)serine, 3-(2-thiazolyl)alanine, 3-(4-thiazolyl)alanine, 3-(1,2,4-triazol-1-yl)-alanine, 3-(1,2,4-triazol-3-yl)-alanine, hexafluorovaline, 4,4,4-trifluorovaline, 3-fluorovaline, 5,5,5-trifluoroleucine, 2-amino-4,4,4-trifluorobutyric acid, 3-chloroalanine, 3-fluoroalanine, 2-amino-3-fluorobutyric acid, 3-fluoronorleucine, 4,4,4-trifluorothreonine, L-allylglycine, tert-Leucine, propargylglycine, vinylglycine, S-methylcysteine, cyclopentylglycine, cyclohexylglycine, 3-hydroxynorvaline, 4-azaleucine, 3-hydroxyleucine, 2-amino-3-hydroxy-3-methylbutanoic acid, 4-thiaisoleucine, acivicin, ibotenic acid, quisqalic acid, 2-indanylglycine, 2-aminoisobutyric acid, 2-cyclobutyl-2-phenylglycine, 2-isopropyl-2-phenylglycine, 2-methylvaline, 2,2-diphenylglycine, 1-amino-1-cyclopropanecarboxylic acid, 1-amino-1-cyclopentanecarboxylic acid, 1-amino-1-cyclohexanecarboxylic acid, 3-amino-4,4,4-trifluorobutyric acid, 3-phenylisoserine, 3-amino-2-hydroxy-5-methylhexanoic acid, 3-amino-2-hydroxy-4-phenylbutyric acid, 3-amino-3-(4-bromophenylpropionic acid, 3-amino-3-(4-chlorophenyl)propionic acid, 3-amino-3-(4-methoxyphenyl)propionic acid, 3-amino-3-(4-fluorophenyl)propionic acid, 3-amino-3-(2-fluorophenyl)propionic acid, 3-amino-3-(4-nitrophenyl)propionic acid, and 3-amino-3-(1-naphthyl)propionic acid.

Certain of the compounds may exist as stereoisomers including optical isomers. The invention includes the use of all stereoisomers and both the racemic mixtures of such stereoisomers as well as the individual enantiomers that may be separated according to methods that are well known to those of ordinary skill in the art.

Compositions of the present invention also comprise caspase inhibitors that are antisense nucleic acids (see, e.g., U.S. Pat. No. 6,492,170 to Watt et al.) or small double-stranded interfering RNA (RNA interference (RNAi)). In one embodiment, antisense nucleic acids or RNAi are used to modulate the expression of a nucleic acid molecule encoding a caspase, thereby modulating the amount of caspase produced. This is accomplished by providing antisense or RNAi compounds that specifically hybridize with one or more target nucleic acids encoding one or more caspases of interest. As used herein, the term “target nucleic acid” encompasses DNA encoding a caspase of interest, RNA (including pre-mRNA and mRNA) transcribed from such DNA, and also cDNA derived from such RNA. The specific hybridization of an antisense nucleic acid with its target nucleic acid interferes with the normal function of the nucleic acid. This modulation of function of a target nucleic acid by compounds which specifically hybridize to the target nucleic acid is generally referred to as “antisense”. The functions of DNA to be interfered with include, but are not limited to, replication and transcription. The functions of RNA to be interfered with include, but are not limited to, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the caspase of interest. In the context of the present invention, “modulation” means a decrease (inhibition) in the expression of a gene. Methods for production and delivery of antisense nucleic acids and RNAi is known in the art (e.g., as taught in PCT Publication WO 2004/016229).

The compositions of the present invention also include caspase inhibitors that are dominant negative caspase proteins. Several caspase genes (Caspase-1, -2, -3, -6, -7 and -8) are expressed as multiple isoforms, due to alternative splicing of the primary transcript. Among those isoforms are enzymatically-inactive variants which are expressed as modified mRNAs or truncated proteins. Such variants may play a crucial role in the regulation of caspase activity. Caspase-8, for example, is expressed in at least seven isoforms. One caspase 8 isoform (MACH alpha-3 isoform) has been shown to effect the activity of the active caspase-8 enzyme in a dominant negative manner, and expression of this isoform inhibited Fas-mediated apoptosis (Boldin et al., Cell 85:803-815 (1996)).

Compositions comprising FLICE-inhibitory proteins (FLIPs), are also provided by the present invention. Inhibition of caspase activity has been attributed to v-FLIPs (viral FLICE-inhibitory proteins), which are present in several gamma-herpes viruses and in molluscipox viruses. v-FLIPs contain two death effector domains (DEDs) and can interfere with the Caspase-8 interaction with the protein FADD, thereby inhibiting the recruitment and activation of Caspase-8 during Fas-mediated apoptosis. The identification of v-FLIPS led to the search for cellular homologs and resulted in the isolation of the protein FLIP (Irmler et al., Nature 388:190-195 (1997)), also known as CASH, Casper, I-FLICE, CLARP, FLAME-1, MRIT and Usurpin (see Rasper et al., Cell Death and Diff. 5:271-288 (1998)). FLIP interacts with FADD via its DEDs and also forms heterodimers with Caspase-8. FLIP is cleaved by Caspase-8, which may result in a tighter binding of the cleaved FLIP to the Caspase-8 enzyme, thereby blocking its proteolytic activity.

### Other Inhibitors of Apoptosis

Without wishing to be bound by any one theory, it is thought that cell death promotes the release of intracellular HMGB1, as well as other pro-inflammatory cytokines and intracellular proteins, to the extracellular environment. Release of these proteins following cell death can lead to the induction of an inflammatory response. As used herein, cell death refers to any type of cell death that causes release of intracellular proteins to the extracellular environment.

Thus, it is believed that apoptosis leads to the release of intracellular HMGB1 to the extracellular environment, thereby inducing an inflammatory response. Such an inflammatory response can be inhibited with compositions comprising one or more inhibitors of apoptosis. As described herein, in one embodiment, the one or more inhibitors of apoptosis are caspase inhibitors. In another embodiment, the one or more inhibitors of apoptosis include, but are not limited to, granzyme inhibitors, Bax inhibitors, Fas/FasL antagonists, inhibitors of apoptosome complex formation, and inhibitors of p53-mediated apoptosis.

### Treatment of Inflammatory Conditions

The present invention provides a method of treating an inflammatory condition in an individual, or treating an individual at risk for having an inflammatory condition, comprising administering to the individual an effective amount of a combination therapy composition as described herein. As used herein, an “effective amount” is an amount sufficient to prevent or decrease an inflammatory response, and/or to ameliorate and/or decrease the longevity of symptoms associated with an inflammatory response. Methods for determining whether a combination therapy composition inhibits an inflammatory condition are known to one skilled in the art. Inhibition of the release of a proinflammatory cytokine from a cell can be measured by any method known to one of skill in the art, for example, using an L929 cytotoxicity assay as is known in the art. The inflammatory condition can be one in which the inflammatory cytokine cascade is activated. In one embodiment, the inflammatory cytokine cascade causes a systemic reaction, such as with endotoxic shock. In another embodiment, the inflammatory condition is mediated by a localized inflammatory cytokine cascade, as in rheumatoid arthritis. Other nonlimiting examples of inflammatory conditions that can be usefully treated using the present invention include those described herein.

In one embodiment, the condition to be treated is selected from one or more of the group consisting of sepsis, peritonitis, pancreatitis, inflammatory bowel disease, ileus, ulcerative colitis, Crohn's disease, ischemia, for example, myocardial ischemia, organic ischemia, or reperfusion ischemia, cachexia, burns, adult respiratory distress syndrome, multiple sclerosis, atherosclerosis, restenosis, arthritis, rheumatoid arthritis, asthma, lupus, adult respiratory distress syndrome, chronic obstructive pulmonary disease, psoriasis, Behcet's syndrome, psoriasis, allograft rejection and graft-versus-host disease. Where the condition is allograft rejection, the composition can advantageously also include an immunosuppressant that is used to inhibit allograft rejection, such as cyclosporin.

Preferably the combination therapy compositions are administered to a patient in need thereof in an amount sufficient to inhibit release of one or more proinflammatory cytokines from a cell and/or to treat an inflammatory condition. In one embodiment, release of the proinflammatory cytokine is inhibited by at least 10%, 20%, 25%, 50%, 75%, 80%, 90%, or 95%, as assessed using methods described herein or other methods known in the art.

The terms “therapy,” “therapeutic,” and “treatment” as used herein, refer to ameliorating symptoms associated with a disease or condition, for example, an inflammatory disease or an inflammatory condition, including preventing or delaying the onset of the disease symptoms, and/or lessening the severity or frequency of symptoms of the disease or condition. The terms “subject” and “individual” are defined herein to include animals such as mammals, including, but not limited to, primates, cows, sheep, goats, horses, dogs, cats, rabbits, guinea pigs, rats, mice or other bovine, ovine, equine, canine, feline, rodent, or murine species. In one embodiment, the animal is a human.

A pharmaceutically acceptable carrier will usually be included with the combination therapy compositions and can be based on the expected route of administration of the composition in therapeutic applications. The route of administration of the composition depends on the condition to be treated. For example, intravenous injection may be preferred for treatment of a systemic disorder such as endotoxic shock, and oral administration may be preferred to treat a gastrointestinal disorder such as a gastric ulcer. The dosage of the combination therapy compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Typically, an effective amount can range from 0.01 mg per day to about 100 mg per day for an adult. Preferably, the dosage ranges from about 1 mg per day to about 100 mg per day or from about 1 mg per day to about 10 mg per day. Depending on the condition, the combination therapy composition can be administered orally, parenterally, intranasally, vaginally, rectally, lingually, sublingually, buccally, intrabuccally and/or transdermally to the patient.

Accordingly, combination therapy compositions designed for oral, lingual, sublingual, buccal and intrabuccal administration can be made without undue experimentation by means well known in the art, for example, with an inert diluent or with an edible carrier. The combination therapy composition may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums, and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, corn starch, and the like. Examples of lubricants include magnesium stearate and potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The combination therapy compositions of the present invention can be administered parenterally, such as, for example, by intravenous, intramuscular, intrathecal and/or subcutaneous injection. Parenteral administration can be accomplished by incorporating the combination therapy compositions of the present invention into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic.

Rectal administration includes administering the combination therapy composition into the rectum and/or large intestine. This can be accomplished using suppositories and/or enemas. Suppository formulations can be made by methods known in the art. For example, suppository formulations can be prepared by heating glycerin to about 120° C., dissolving the combination therapy composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves, and the like.

The combination therapy compositions of the present invention can be administered nasally to a patient. As used herein, nasally administering or nasal administration includes administering the combination therapy compositions to the mucous membranes of the nasal passage and/or nasal cavity of the patient. Pharmaceutical compositions for nasal administration of a composition include therapeutically effective amounts of the combination therapy composition prepared by well-known methods to be administered, for example, as a nasal spray, nasal drop, suspension, gel, ointment, cream and/or powder. Administration of the composition may also take place using a nasal tampon and/or nasal sponge.

The combination therapy compositions described herein can also include one or more additional agents used to treat an inflammatory condition. Such agents are known to one of skill in the art. The agent may be, for example, an antagonist or inhibitor of an early sepsis mediator. As used herein, an early sepsis mediator is a proinflammatory cytokine that is released from cells soon (i.e., within 30-60 min.) after induction of an inflammatory cytokine cascade (e.g., exposure to LPS). Nonlimiting examples of these cytokines are IL-lot, IL 0, IL-6, PAF, and MIF. Also included as early sepsis mediators are receptors for these cytokines (for example, tumor necrosis factor receptor type 1) and enzymes required for production of these cytokines (for example, interleukin-1β converting enzyme). Antagonists or inhibitors of any early sepsis mediator, now known or later discovered, can be useful for these embodiments by further inhibiting an inflammatory cytokine cascade.

Nonlimiting examples of antagonists of early sepsis mediators are antisense compounds that bind to the mRNA of the early sepsis mediator, preventing its expression (see, e.g., Ojwang et al., Biochemistry 36:6033-6045, 1997; Pampfer et al., Biol. Reprod. 52:1316-1326, 1995; U.S. Pat. No. 6,228,642; Yahata et al., Antisense Nucleic Acid Drug Dev. 6:55-61, 1996; and Taylor et al., Antisense Nucleic Acid Drug Dev. 8:199-205, 1998), ribozymes that specifically cleave the mRNA of the early sepsis mediator (see, e.g., Leavitt et al., Antisense Nucleic Acid Drug Dev. 10:409-414, 2000; Kisich et al., 1999; and Hendrix et al., Biochem. J. 314 (Pt. 2):655-661, 1996), and antibodies that bind to the early sepsis mediator and inhibit their action (see, e.g., Kam and Targan, Expert Opin. Pharmacother. 1:615-622, 2000; Nagahira et al., J. Immunol. Methods 222:83-92, 1999; Lavine et al., J. Cereb. Blood Flow Metab. 18:52-58, 1998; and Holmes et al., Hybridoma 19:363-367, 2000). An antagonist or inhibitor of an early sepsis mediator, now known or later discovered, is envisioned as within the scope of the invention. The skilled artisan can determine the amount of early sepsis mediator to use in these compositions for inhibiting any particular inflammatory cytokine cascade without undue experimentation, e.g., using routine dose-response studies.

Other agents that can be administered with the combination therapy compositions described herein include, e.g., Vitaxin™ and other antibodies targeting αvβ3 integrin (see, e.g., U.S. Pat. No. 5,753,230, PCT Publication Nos. WO 00/78815 and WO 02/070007; the entire teachings of all of which are incorporated herein by reference) and anti-IL-9 antibodies (see, e.g., PCT Publication No. WO 97/08321; the entire teachings of which are incorporated herein by reference).

In one embodiment, the combination therapy compositions of the invention are administered with inhibitors of TNF biological activity. Such inhibitors of TNF activity include, e.g., peptides, proteins, synthesized molecules, for example, synthetic organic molecules, naturally-occurring molecule, for example, naturally occurring organic molecules, nucleic acid molecules, and components thereof. Preferred examples of agents that inhibit TNF biological activity include infliximab (Remicade; Centocor, Inc., Malvern, Pa.), etanercept (Immunex; Seattle, Wash.), adalimumab (D2E7; Abbot Laboratories, Abbot Park Ill.), CDP870 (Pharmacia Corporation; Bridgewater, N.J.) CDP571 (Celltech Group plc, United Kingdom), Lenercept (Roche, Switzerland), and Thalidomide.

### EXEMPLIFICATION

### Example 1

### HMGB1 Functions Downstream of Pro-apoptotic Factors During Sepsis-Induced Apoptosis

### Materials and Methods

BALB/c mice were subjected to cecal ligation and puncture (CLP) surgery. At 24 h after surgery, the mice were administered one dose of either mAb 6E6 monoclonal antibody, which recognizes an epitope on the HMGB1 protein, or control IgG, at a concentration of 10 μg/mouse, by intraperitoneal injection. The mice were sacrificed at 40 hours after the completion of surgery and their spleens were dissected. Tissue sections of spleens were produced using a standard protocol involving formalin fixation and paraffin embedding. In particular, spleens and thymus were obtained immediately after euthanasia of the mice, fixed in 10% neutral buffered formalin (Sigma, St. Louis, Mo.) for 24 hours and dehydrated in alcohol and embedded in paraffin. Tissue sections were cut at 5 μm. Sections were mounted on glass slides and subjected to staining using a TUNEL assay, performed according to manufacturer's protocol (ApoAlert DNA fragmentation assay kit, BD BioSciences, Palo Alto, Calif., Cat# K2024-1). Immunostaining was performed using antibodies against the apoptotic markers, Caspase 3 (rabbit anti-human caspase 3 antibody, R & D system, Cat. #AF 835) and armexin VI (goat anti-human annexin VI, San Cruz. Biotechnology Inc., Santa Cruz, Calif., Cat # sc-1931) using Vectastain kits (Vector Lab, Burlingame, Calif., Catalog Nos. PK-4001 and PK-4005, respectively) according to manufacturer's instructions. The slides were mounted by dispensing one drop of VECTASHIELD mounting medium (Vector Lab, Cat# H-10000) on to the section on the slide. A coverslip was subsequently lowered onto the drop of medium, thereby allowing the medium to dispense over the entire section. Slides were imaged using imaged using a Zeiss Axiovert 200 microscope (Gottingen, Germany). Four to five mice were analyzed from each group.

### Results

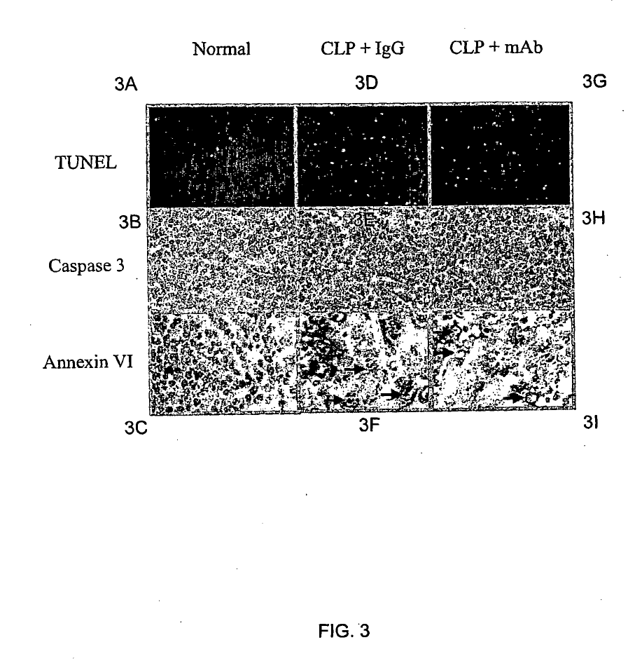

Administration of HMGB1 monoclonal antibodies to septic mice, following CLP surgery, did not inhibit apoptosis in these mice. The effects of the HMGB1 antibody mimicked the effects of control IgG, with respect to the detection of apoptotic markers (FIG. 3). These results indicate that HMGB1 activity does not induce apoptosis, but functions downstream of apoptosis induction.

### Example 2

### Inhibition of Caspase Activity Inhibits Sepsis-Induced Expression of HMGB1Protein in RAW 264.7 Cells

### Materials and Methods

Treatment of RAW 264.7 cells with caspase inhibitor and Western blotting of HMGB1. Murine macrophage-like RAW 264.7 cells (American Type Culture Collection, ATCC, Rockville, Md.) were cultured in a 24-well culture dish in RPMI 1640 medium (Life Technologies, Grand Island, N.Y.), supplemented with 10% fetal bovine serum (Gemini, Catabasas, Calif.), penicillin and streptomycin (Life Technologies). Cells were used at 90% confluence and treatment was carried out in serum-free Opti-MEM I medium (Life Technologies). The cells were treated with LPS (200 ng/ml, Sigma, E. coli. 0111:B4) and various concentrations, ranging from 0.1 to 10 μM, of either the caspase inhibitor, Z-VAD-FMK (BD Biosciences), or a control peptide, Z-FA-FMK (BD Biosciences), in Opti-MEM I medium (Life Technologies, Grand Island, N.Y.). After 16 h, cell culture supernatants were concentrated by Centricon 10 (Millpore Corp., Bedford, Mass.) and HMGB1 levels were determined by Western blot. Briefly, proteins were fractionated by SDS-PAGE, transferred to PVDF Immunoblot membrane (Bio-Rad laboratories, Hercules, Calif.), and probed with polygonal anti-HMGB1 IgG (5 μg/ml) for Western blot. Donkey anti-rabbit IgG conjugated with BP was (Amersham, Little Chalfont Buckinghamshire, England) used at final dilution of 1:5,000. Polyclonal antibodies raised against purified HMGB1 were generated in rabbits (Cocalico Biologicals, Inc., Reamstown, Pa.) and IgG was purified using protein A agarose according to manufacturer's instructions (Pierce, Rockford, Ill.). Western blots were scanned with a silver image scanner (Silverscanner II, Lacie Limited, Beaverton, Oreg.), and the relative band intensity was quantified using the NIH image 1.59 software. The levels of HMGB1 were calculated with reference to standard curves generated with purified HMGB1.

### Immunofluorescence

RAW 264.7 cells were plated in 8-well slide chambers (Lab-Tek, Nalge Nunc Internationals, Naperville, Ill.) and used at 60% confluence. Cells were incubated with LPS (100 ng/ml) alone, or LPS (100 ng/ml) with either 1 μM Z-VAD-FMK (VAD) or Z-FA-FMK control peptide for 16 hours in Opti-MEM I medium. Cells were washed 3 times with phosphate-buffered saline (PBS) at pH 7.2 and fixed in 2% neutral formalin for 20 minutes at room temperature. Polyclonal anti-HMGB1 antibody was added at a concentration of 2.5 μg/ml and incubated for an additional 30 minutes at room temperature. FITC-labeled anti-rabbit IgG was added (1:300 dilution, Sigma, St. Louis, Mo.; Cat. No. F9887). Cells were then washed 3 times with PBS (pH 7.2) with 0.1% saponin (Sigma Cat. #S4521), and mounted for fluorescent confocal microscopy analysis. To mount cells on a slide, one drop of VECTASHIELD mounting medium (Vector Lab, Cat#H-10000) was dispensed on to the section and allowed to cover the entire section. A coverslip was added, and the slides were imaged using a Zeiss, Axiovert 200 microscope (Gottingen, Germany).

### Results

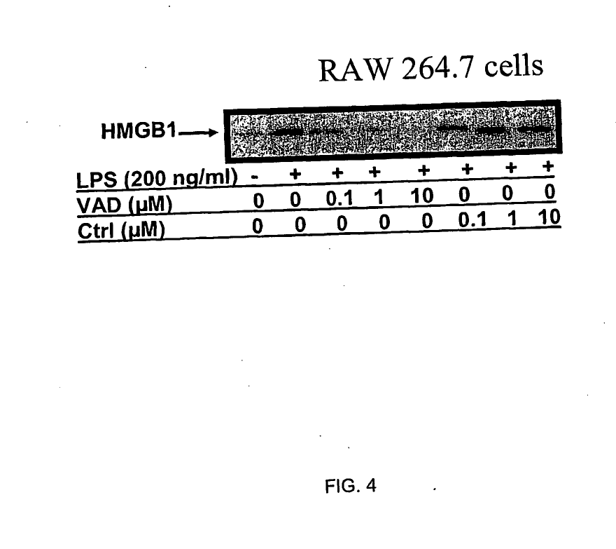

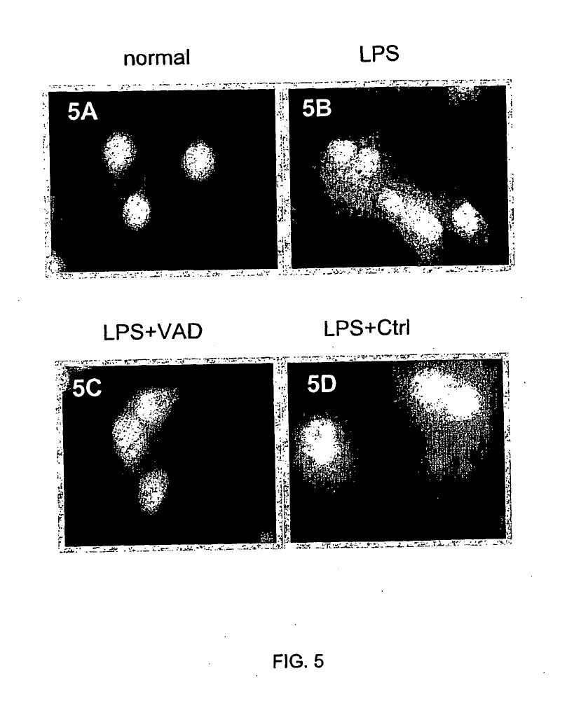

As depicted in FIG. 4, treatment of LPS-stimulated RAW 264.7 cells with the caspase inhibitor, VAD, inhibited LPS-induced expression of HMGB1 protein in a dose-dependent manner (FIG. 4). Treatment of RAW 264.7 cells with the control peptide, Z-FA-FMK, did not have an effect on HMGB1 expression. Immunofluorescence staining indicated that HMGB1 is primarily nuclear in normal RAW 264.7 cells (FIG. 5A). Treatment of these cells with LPS, however, resulted in the translocation of HMGB1 to the cytoplasm (FIG. 5B). Administration of the caspase inhibitor (VAD) (FIG. 5C), but not the control peptide (Ctrl) (FIG. 5D), inhibited HMGB1 translocation to the cytoplasm.

### Example 3

### Apoptosis Induces HMGB1 Expression in Septic Mice

### Materials and Methods

Surgery and Treatment of Mice with Caspase Inhibitor

Cecal ligation and puncture (CLP) surgery was performed on BALB/c mice to induce sepsis as described previously (Fink, M. P. and Heard, S. O., J. Surg. Res. 49:186-96 (1990); Friedman, G., et al., Crit. Care Med. 26:2078-86 (1998); Remick, D., et al., Shock 4:89-95 (1995)). At 90 min after the surgery, 0.5 mg of either Z-VAD-FMK (VAD) caspase inhibitor or Z-FA-FMK control peptide was administered to each mouse by intraperitoneal injection. Mice were euthanized 24 h after CLP.

### Detection of HMGB1 and Cytokine Protein Levels

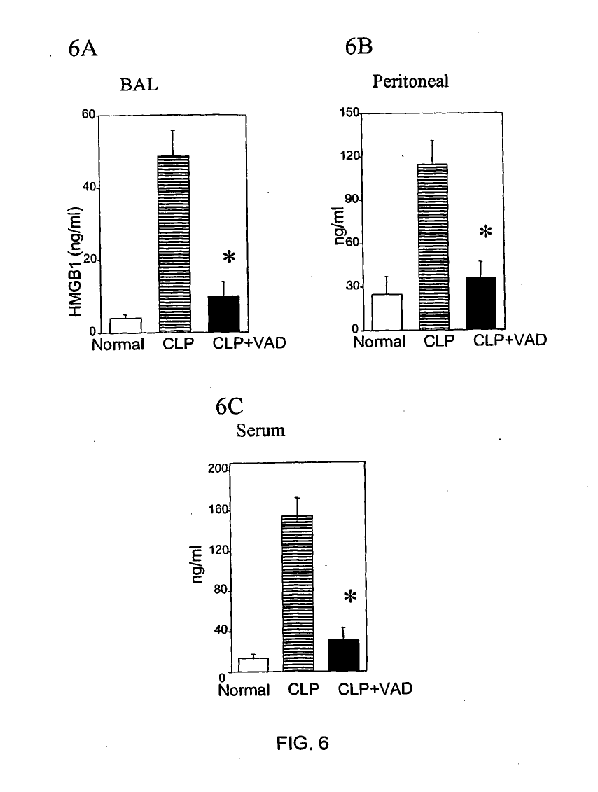

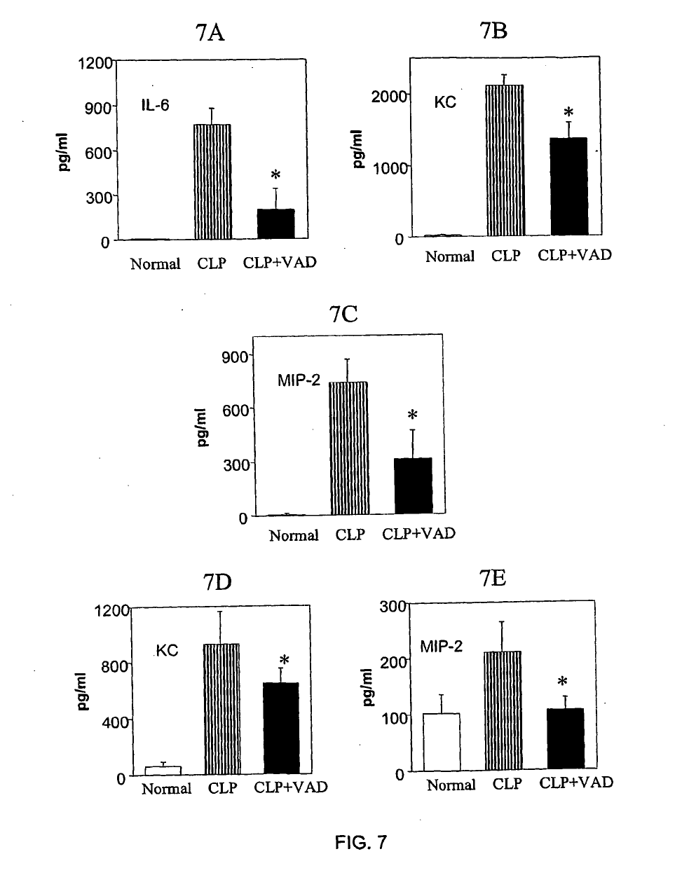

HMGB1 protein levels in bronchoalveolar fluid (BAL), peritoneal fluid and serum were measured (FIG. 6). Protein levels of certain serum (e.g., IL-6, KC, MIP-2) and BAL (e.g., KC, MW-2) cytokines were determined (FIG. 7). Cell-conditioned medium, lung lavage fluid or peritoneal lavage fluid (100-2000) were first concentrated by passing through Centricon 10 (Millpore Corp. Bedford, Mass.). Cytokines were measured by using an enzyme-linked immunosorbent assay (ELISA) kit according to the instructions of the manufacturer (R & D System Inc., Minneapolis, Minn.). To determine HMGB1 concentrations, Centricon 10-filtered samples were fractionated by SDS-PAGE, transferred to PVDF Immunoblot membrane (Bio-Rad laboratories, Hercules, Calif.), and probed with polyclonal anti-HMGB1 IgG (5 μg/ml) for Western blot. Donkey anti-rabbit IgG conjugated with HRP (Amersham, Little Chalfont Buckinghamshire, England) and used at final dilution of 1:5,000. Polyclonal antibodies raised against purified HMGB1 were generated in rabbits (Cocalico Biologicals, Inc., Reamstown, Pa.) and IgG was purified using protein A agarose according to manufacturer's instructions (Pierce, Rockford, Ill.). Western blots were scanned with a silver image scanner (Silverscanner II, Lacie Limited, Beaverton, Oreg.), and the relative band intensity was quantified by using the NIH image 1.59° software. The levels of HMGB1 were calculated with reference to standard curves generated with purified HMGB1.

### Immunofluorescence

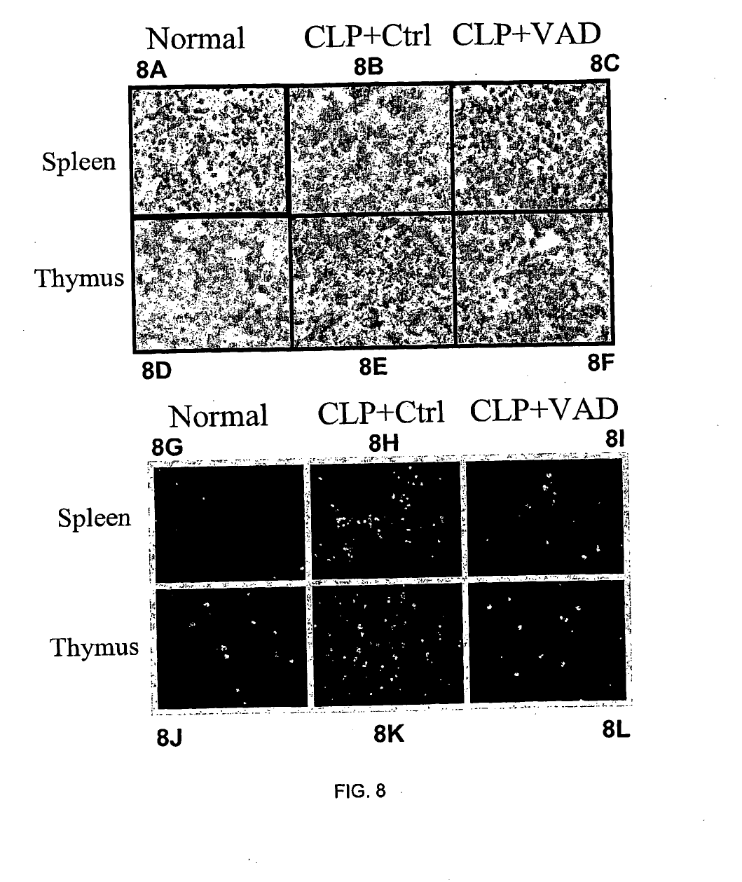

Apoptotic cells were detected by performing Caspase 3 staining (FIG. 8A) and TUNEL staining (FIG. 8B) on fixed spleen and thymus sections from sacrificed mice that were either untreated (Normal) or treated with Z-VAD-FMK (VAD) caspase inhibitor (CLP+VAD) or Z-FA-FMK control peptide (CLP+ctrl) following CLP surgery. Seven mice were analyzed in each group. Tissue sections were cut at 5 μm. Sections were placed on glass slides and subjected to staining using a TUNEL assay, performed according to manufacturer's protocol (ApoAlert DNA fragmentation assay kit, BD BioSciences, Palo Alto, Calif., Cat# K2024-1). Immunostaining was performed using antibodies against the apoptotic markers, Caspase 3 (rabbit anti-human caspase 3 antibody, R & D system, Cat. #AF 835) and annexin VI (goat anti-human annexin VI, San Cruz. Biotechnology Inc., Santa Cruz, Calif., Cat. #sc-1931) using Vectastin kits (Vector Lab, Burlingame, Calif., Catalog Nos. PK4001 and PK-4005, respectively) according to manufacturer's instructions. The slides were mounted by dispensing one drop of VECTASHIELD mounting medium (Vector Lab, Cat# H-10000) on to the section on the slide. A coverslip was subsequently lowered onto the drop of medium, thereby allowing the medium to dispense over the entire section. Slides were imaged using imaged using a Zeiss Axiovert 200 microscope (Gottingen, Germany). Four to five mice were analyzed from each group.

### Results

CLP-induced sepsis resulted in increased levels of HMGB1 (FIG. 6) and the cytokines, IL-6, KC and MIP-2 (FIG. 7), in the BAL fluid, peritoneal fluid and serum of septic mice. Treatment of septic mice with the caspase inhibitor, VAD, inhibited expression of each of these pro-inflammatory factors. Furthermore, treatment with VAD also reduced the number of apoptotic cells detected in spleen and thymus sections from CLP-induced septic mice (FIG. 8). These results indicate that induction of apoptosis following sepsis results in increased protein levels of HMGB1 and various pro-inflammatory cytokines.

### Example 4

### Sepsis-Induced Apoptosis Promotes the Release of HMGB1 Protein from RAW 264.7 Cells

### Materials and Methods

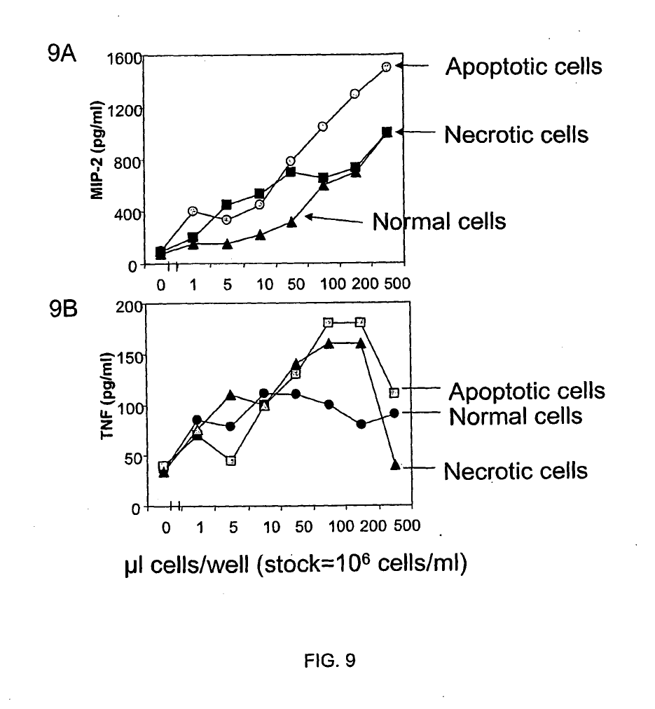

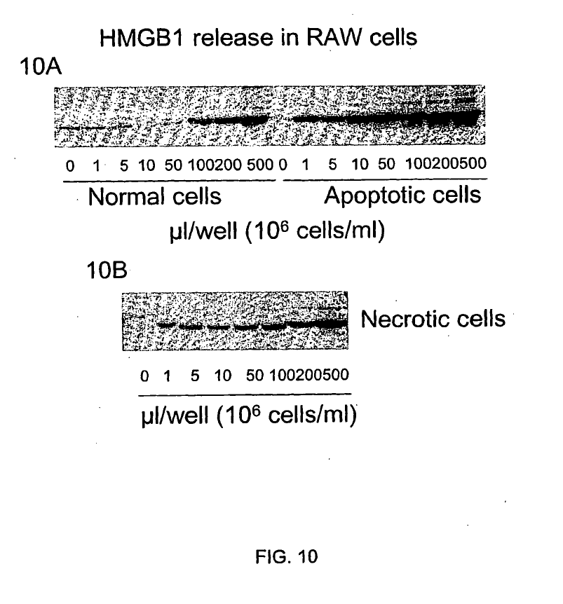

RAW 264.7 cells (American Type Culture Collection, ATCC, Rockville, Md.) were cultured in RPMI 1640 medium (Life Technologies, Grand Island, N.Y.) supplemented with 10% fetal bovine serum (Gemini, Catabasas, Calif.), penicillin and streptomycin (Life Technologies) in a 37° C. humidified incubator with 5% CO₂ and 95% room air. Cells were used at 90% confluence and treatment was carried out in serum-free Opti-MEM I medium (Life Technologies). The cells were either untreated (Normal cells), treated with 6 μM Camptothein (Sigma) for 6 hours to induce apoptosis (Apoptotic cells), or subjected to three cycles of freezing and thawing to induce necrosis (Necrotic cells). Cells of each treatment were scraped off of the plate in Optimem I medium and were added to RAW 264.7 cells in 24-well plates in order to stimulate the cells. Various amounts of treated cells, ranging from 0 to 500 μl of cells (at a concentration of approximately 10⁶ cells/ml), were added to each well and were incubated for 16 hours to determine what effect the differentially-treated cell populations had on the release of MW-2, TNF and HMGB1 from normal RAW 264.7 cells. Following the incubation, supernatants were harvested from each sample of cells and measured for TNF and MIP-2 release (FIG. 9) or HMGB1 release (FIG. 10). INF and MIP-2 levels were measured by enzyme-linked immunosorbent assay (ELISA) kit according to the instructions of the manufacturer (R & D System Inc., Minneapolis, Minn.). Cell-conditioned medium (100-200 μl) were first concentrated by passing through Centricon 10 (Millpore Corp. Bedford, Mass.). Proteins were fractionated by SDS-PAGE, transferred to PVDF Immunoblot membrane (Bio-Rad laboratories, Hercules, Calif.), and probed with polyclonal anti-HMGB1 IgG (5 μg/ml) for Western blot. Donkey anti-rabbit IgG conjugated with HRP (Amersham, Little Chalfont Buckinghamshire, England) was used at final dilution of 1:5,000. Polyclonal antibodies raised against purified HMGB1 were generated in rabbits (Cocalico Biologicals, Inc., Reamstown, Pa.) and IgG was purified using protein A agarose according to manufacturer's instructions (Pierce, Rockford, Ill.). Western blots were scanned with a silver image scanner (Silverscanner II, Lacie Limited, Beaverton, Oreg.), and the relative band intensity was quantified by using the NIH image 1.59 software. The levels of HMGB1 were calculated with reference to standard curves generated with purified HMGB1.

### Results

Incubation of RAW 264.7 cells with apoptotic cells that had been treated with Camptothein increased the amount of MIP-2, TNF and HMGB1 released from RAW 264.7 cells, relative to the amount of MIP-2, TNF and HMGB1 released from RAW 264.7 cells that were incubated with untreated control cells (FIGS. 9 and 10). Incubation with necrotic cells had an intermediate effect. These results indicate that induction of apoptosis triggers the release of HMGB1 and the cytokines MIP-2 and TNF, suggesting that pro-apoptotic factors function upstream of these pro-inflammatory agents during sepsis.

The relevant teachings of all publications cited herein not previously incorporated by reference, are incorporated herein by reference in their entirety. While this invention has been particularly shown and described with references to preferred embodiments thereof, it will be understood by those skilled in the art that various changes may be made therein without departing from the scope of the invention encompassed by the appended claims.

1. A composition comprising an agent that inhibits HMGB biological activity and a caspase inhibitor.

2. The composition of claim 1, wherein said agent that inhibits HMGB biological activity inhibits one or more activities selected from the group consisting of: inhibiting HMGB receptor binding; inhibiting HMGB signaling; inhibiting HMGB-mediated inflammation; and inhibiting HMGB-mediated release of a proinflammatory cytokine.

3-4. (canceled)

5. A composition comprising an agent that inhibits HMGB biological activity and a caspase inhibitor, wherein said agent that inhibits HMGB biological activity is selected from the group consisting of: an HMGB A box or a biologically active fragment thereof; an antibody that binds to an HMGB polypeptide or an antigen-binding fragment thereof; an HMGB small molecule inhibitor; a cholinergic agonist; an antibody to TLR2 or an antigen-binding fragment thereof; a soluble TLR2 polypeptide; an antibody to RAGE or an antigen-binding fragment thereof; a soluble RAGE polypeptide and a RAGE small molecule antagonist.

6. The composition of claim 1, wherein said agent that inhibits HMGB biological activity is selected from the group consisting of an HMGB A box or biologically active fragment thereof, an antibody that binds to an HMGB polypeptide or an antigen-binding fragment thereof, an HMGB small molecule inhibitor, and a cholinergic agonist.

7-19. (canceled)

20. The composition of claim 6, wherein said antibody that binds to an HMGB polypeptide or antigen-binding fragment thereof is selected from the group consisting of a monoclonal antibody, an antigen-binding fragment of a monoclonal antibody, a chimeric antibody, an antigen-binding fragment of a chimeric antibody, a humanized antibody, an antigen-binding fragment of a humanized antibody, a human antibody, and an antigen-binding fragment of a human antibody.

21-24. (canceled)

25. The composition of claim 6, wherein said HMGB small molecule inhibitor is an ester of an alpha-ketoalkanoic acid.

26. The composition of claim 25, wherein said ester of an alpha-ketoalkanoic acid is selected from the group consisting of an ester of a C3 to C8 straight chain or branched alpha-ketoalkanoic acid, an ester of pyruvic acid, an ethyl ester, a propyl ester, a butyl ester, a carboxymethyl ester, an acetoxymethyl ester, a carbethoxymethyl ester, an ethoxymethyl ester, and ethyl pyruvate.

27-38. (canceled)

39. The composition of claim 1, wherein said caspase inhibitor is selected from the group consisting of carobenzoxyl-valyl-alanyl-aspartyl-[O-methyl]-fluoromethylketone (Z-VAD-FMK) or a derivative thereof, a peptide or modified derivative thereof, an inhibitor of caspase-2, an inhibitor of caspase-3, an inhibitor of caspase-6, an inhibitor of caspase-7, an inhibitor of caspase-7, an inhibitor of caspase-9, an inhibitor of caspase-10, a small molecule caspase inhibitor, an inhibitor of apoptosis (IAP) family member protein, M-920, M-791, acetyl-Ile-Gln-Ala-Cys-Arg-Gly (Ac-IQACRG) (SEQ ID NO:46), carbobenzoxyl-valyl-lysyl(biotin)-aspartyl(O-methyl)-fluoromethylketone (Z-VKD-FMK), BD™ ApoBlock Caspase Inhibitor, a discoharbdin compound, AC-AAVALLPAVLLALLALPIETD-CHO (SEQ ID NO:62), a FLICE-inhibitory protein (FLIP), a dominant negative caspase, and an antisense nucleic acid that is capable of hybridizing to a target nucleic acid, such that hybridization of said antisense nucleic acid to said target nucleic acid inhibits expression of one or more cellular caspases.

40. (canceled)

41. The composition of claim 39, wherein said peptide or modified derivative thereof is a peptide having an amino acid sequence selected from the group consisting of WEHD, YVAD, IETD, AEVD, LEED, LEVD, YVKD, DEVD, DMQD, DQTD, VEID, IETD, LEHD, AEVD, VDVAD, and LDESD.

42. (canceled)

43. The composition of claim 39, wherein the peptide or modified derivative thereof comprises one or more modifications selected from the group consisting of: a fluoromethyl ketone (FMK) group, a chloromethyl ketone (CMK) group, an aldehyde (CHO) group, a nitrile group, a ketone group, a thiomethylketone group, a benzyloxycarbonyl (BOC) group, a carbobenzoxyl (Z) group, an acetyl (Ac) group and an O-methyl (OCH₃) group.

44-46. (canceled)

47. The composition of claim 39 wherein said small molecule caspase inhibitor is selected from the group consisting of a gamma-ketoacid dipeptide, a pyrazinone, Z-Asp-[(2,6-dichlorobenzoyl)oxy]methane, 2,2′-Methylenebis(1,3-cyclohexanedione), 5-[(S)-(−)-2-(Methoxymethyl)pyrrolidino]sulfonylisatin, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII and formula XXVIII.

48-62. (canceled)

63. A method of treating an inflammatory condition in a patient comprising administering to said patient a pharmaceutical composition comprising an agent that inhibits HMGB biological activity and a caspase inhibitor, wherein said agent that inhibits HMGB biological activity is selected from the group consisting of: an HMGB A box or a biologically active fragment thereof; an antibody that binds to an HMGB polypeptide or an antigen-binding fragment thereof; an HMGB small molecule inhibitor; a cholinergic agonist; an antibody to TLR2 or an antigen-binding fragment thereof; a soluble TLR2 polypeptide; an antibody to RAGE or an antigen-binding fragment thereof; a soluble RAGE polypeptide and a RAGE small molecule antagonist.

64. The method of claim 63, wherein said agent that inhibits HMGB biological activity is selected from the group consisting of an HMGB A box or biologically active fragment thereof, an antibody that binds to an HMGB polypeptide or an antigen-binding fragment thereof, an HMGB small molecule inhibitor and a cholinergic agonist.

65-77. (canceled)

78. The method of claim 64, wherein said antibody that binds to an HMGB polypeptide or antigen-binding fragment thereof is selected from the group consisting of a monoclonal antibody, an antigen-binding fragment of a monoclonal antibody, a chimeric antibody, an antigen-binding fragment of a chimeric antibody, a humanized antibody, an antigen-binding fragment of a humanized antibody, a human antibody and an antigen-binding fragment of a human antibody.

79-82. (canceled)

83. The method of claim 64, wherein said HMGB small molecule inhibitor is an ester of an alpha-ketoalkanoic acid.

84. The method of claim 83, wherein said ester of an alpha-ketoalkanoic acid is selected from the group consisting of an ester of a C3 to C8 straight chain or branched alpha-ketoalkanoic acid, an ester of pyruvic acid, an ethyl ester, a propyl ester, a butyl ester, a carboxymethyl ester, an acetoxymethyl ester, a carbethoxymethyl ester, an ethoxymethyl ester, and ethyl pyruvate.

85-96. (canceled)

97. The method of claim 63, wherein said caspase inhibitor is selected from the group consisting of carobenzoxyl-valyl-alanyl-aspartyl-[O-methyl]-fluoromethylketone (Z-VAD-FMK) or a derivative thereof, a peptide or modified derivative thereof, an inhibitor of caspase-2, an inhibitor of caspase-3, an inhibitor of caspase-6, an inhibitor of caspase-7, an inhibitor of caspase-7, an inhibitor of caspase-9, an inhibitor of caspase-10, a small molecule caspase inhibitor, an inhibitor of apoptosis (IAP) family member protein, M-920, M-791, acetyl-Ile-Gln-Ala-Cys-Arg-Gly (Ac-IQACRG) (SEQ ID NO:46), carbobenzoxyl-valyl-lysyl(biotin)-aspartyl(O-methyl)-fluoromethylketone (Z-VKD-FMK) BD™ ApoBlock Caspase Inhibitor, a discoharbdin compound, AC-AAVALLPAVLLALLALPIETD-CHO (SEQ ID NO:62), a FLICE-inhibitory protein (FLIP), a dominant negative caspase, and an antisense nucleic acid that is capable of hybridizing to a target nucleic acid, such that hybridization of said antisense nucleic acid to said target nucleic acid inhibits expression of one or more cellular caspases.

98. (canceled)

99. The method of claim 97, wherein said peptide or modified derivative thereof is a peptide having an amino acid sequence selected from the group consisting of WEHD, YVAD, IETD, AEVD, LEED, LEVD, YVKD, DEVD, DMQD, DQTD, VEID, IETD, LEHD, AEVD, VDVAD, and LDESD.

100. (canceled)

101. The method of claim 97, wherein the peptide or modified derivative thereof comprises one or more modifications selected from the group consisting of: a fluoromethyl ketone (FMK) group, a chloromethyl ketone (CMK) group, an aldehyde (CHO) group, a nitrile group, a ketone group, a thiomethylketone group, a benzyloxycarbonyl (BOC) group, a carbobenzoxyl (Z) group, an acetyl (Ac) group and an O-methyl (OCH₃) group.

102-104. (canceled)

105. The method of claim 97, wherein said small molecule caspase inhibitor is selected from the group consisting of a gamma-ketoacid dipeptide, a pyrazinone, Z-Asp-[(2,6-dichlorobenzoyl)oxy]methane, 2,2′-Methylenebis(1,3-cyclohexanedione), 5-[(S)-(−)-2-(Methoxymethyl)pyrrolidino]sulfonylisatin, formula VII, formula VIII, formula IX, formula X, formula XI, formula XII, formula XIII, formula XIV, formula XV, formula XVI, formula XVII, formula XVIII, formula XIX, formula XX, formula XXI, formula XXII, formula XXIII, formula XXIV, formula XXV, formula XXVI, formula XXVII and formula XXVIII.

106-119. (canceled)
